(19) United States
(12) Patent Application Publication
(10) Pub. No.: US 2014/0072654 A1
(43) Pub. Date: Mar. 13, 2014

(54) CANCER WITH METABOLIC THERAPY AND HYPERBARIC OXYGEN

(71) Applicants: Dominic Paul D'Agostino, Tampa, FL (US); Patrick Arnold, Champaign, IL (US); Angela Marie Poff, Brandon, FL (US)

(72) Inventors: Dominic Paul D'Agostino, Tampa, FL (US); Patrick Arnold, Champaign, IL (US); Angela Marie Poff, Brandon, FL (US)

(73) Assignees: SAVIND, INC., Seymour, IL (US); UNIVERSITY OF SOUTH FLORIDA, Tampa, FL (US)

(21) Appl. No.: 14/078,044

(22) Filed: Nov. 12, 2013

Related U.S. Application Data
(63) Continuation-in-part of application No. PCT/US2012/037099, filed on Jun. 21, 2012.
(60) Provisional application No. 61/579,779, filed on Dec. 23, 2011.
(60) Provisional application No. 61/730,813, filed on Nov. 28, 2012.

(51) Int. Cl. A61K 31/121; A61K 33/00

(52) U.S. Cl. CPC A61K 31/121; A61K 33/00
USPC 424/613

(57) ABSTRACT

The present invention demonstrates the therapeutic use of ketone esters for seizure disorders, Alzheimer's disease malignant brain cancer, and other cancers, which are associated with metabolic dysregulation. The administration of a ketogenic diet, such as ketone esters, while concurrently subjecting the patient to a hyperbaric, oxygen-enriched environment resulted in therapeutic ketosis. Optionally, the hyperbaric, oxygen-enriched environment is 100% oxygen at 2.5 ATA absolute. The ketone esters may be derived from acetoacetate and can include R,S-1,3-butanediol acetoacetate monoester, R,S-1,3-butanediol acetoacetate diester, or a combination of the two. The treatment may further include administering at least 10% ketone supplementation, such as acetoacetate, adenosine monophosphate kinase, 1,3-butanediol, or ketone ester, to the patient.

### CROSS REFERENCE TO RELATED APPLICATION

This application is a continuation-in-part of PCT/US2012/037099, entitled “The Use of Keone Esters for Prevention of CNS Oxygen Toxicity”, filed on Jun. 21, 2012, which claims priority to U.S. Provisional Application No. 61/483,927 entitled “The Use of Ketone Esters for Prevention of CNS Oxygen Toxicity”, filed May 9, 2011 and U.S. Provisional Application No. 61/579,779 entitled “The Use of Ketone Esters for Prevention of CNS Oxygen Toxicity”, filed Dec. 23, 2011; and which claims priority to U.S. Provisional Application No. 61/730,813 entitled “Targeting Cancer with Metabolic Therapy and Hyperbaric Oxygen”, filed Nov. 28, 2012, the contents of each of which are hereby incorporated by reference into this disclosure.

### FIELD OF INVENTION

This invention relates to methods of treating cancers and oncogenic diseases. Specifically, the invention provides a novel method of targeting cancerous tissues using hyperbaric oxygen and ketone-based metabolic therapy.

### BACKGROUND OF THE INVENTION

Despite decades of intensive research, cancer remains the second leading cause of death in the United States. One in every two men and one in three women will develop cancer in their lifetime, with one in four men and one in five women dying from cancer. Though cancer has shown a slow decline since early 1990's, in part due to early detection, preventative measures, decreased tobacco use, advances in the field have done little to improve the survival outcome of patients with late-stage metastatic cancer. Standard care typically involves surgery, chemotherapy, and radiation, but these treatments often cause toxic side effects and may even promote cancer progression and metastasis (Sun, et al. (2012) Treatment-induced damage to the tumor microenvironment promotes prostate cancer therapy resistance through WNT16B. Nature medicine; Seyfried, et al. (2010) Does the existing standard of care increase glioblastoma energy metabolism? The lancet oncology 11: 811-813). While many primary tumors can be controlled with conventional therapies, these treatments are largely ineffective against long-term management of metastatic disease (Graeme, et al. (2004) The contribution of cytotoxic chemotherapy to 5-year survival in adult malignancies. Clinical Oncology 16).

Metastasis is a complex phenomenon in which cancer cells spread from a primary tumor to establish foci in a distal tissue. The specific changes which mediate metastasis remain unclear; however, the process generally involves local tumor growth, invasion through the basement membrane and surrounding tissue, intravasation into the blood vessels, dissemination and survival in circulation, extravasation from the vasculature, and re-establishment of tumors at distal tissues. As metastasis is responsible for over 90 percent of cancer-related deaths, there is a substantial need for novel treatments effective against metastatic cancer (Gupta & Massagué (2006) Cancer metastasis: building a framework. Cell 127: 679-695). While many primary tumors can be controlled with conventional therapies like surgery, chemotherapy, and radiation, these treatments are often ineffective against long-term management of metastatic disease which is responsible for 90 percent of cancer-related deaths (Graeme, et al. (2004) The contribution of cytotoxic chemotherapy to 5-year survival in adult malignancies. Clinical Oncology 16; Gupta & Massagué (2006) Cancer metastasis: building a framework. Cell 127: 679-695). There is a substantial need for novel treatments effective against metastatic cancer. The epithelial-to-mesenchymal transition (EMT) is the activation of a latent embryonic program causing a switch from epithelial to mesenchymal phenotype, and alterations in cell-cell/cell-matrix, which enhances cellular motility. Key cellular processes involved in EMT in vitro have been shown to affect metastatic spread in vivo, though metastasis is difficult to study in vivo due to the lack of adequate animal models.

Eighty-eight percent of ATP is made via oxidative phosphorylation in the mitochondria, through an oxygen-dependent pathway. Hypoxic conditions cause a shift to anaerobic fermentation, whereby ATP is produced through substrate level phosphorylation in an oxygen independent pathway. This adaptation to hypoxic mediated fermentation, which is an inefficient process for a rapidly dividing cell, requires HIF-1. Cancer is known to express abnormal energy metabolism characterized by very high rates of aerobic glycolysis (fermentation in the presence of oxygen) (Warburg (1956) On the origin of cancer cells. Science 123: 309-314). This feature is known as The Warburg Effect and is a consequence of mitochondrial dysfunction and genetic mutations within the cancer cell (Seyfried & Shelton (2010) Cancer as a metabolic disease. Nutrition & metabolism 7: 7). This pathway generates lactate, causing an acidic microenvironment which results in invasion and metastasis due to the oncogene and tumor suppressor mutations. The Warburg Effect creates a glucose-dependency which can be targeted therapeutically (Seyfried & Shelton (2010) Cancer as a metabolic disease. Nutrition & metabolism 7: 7; Seyfried, et al. (2008) Targeting energy metabolism in brain cancer with calorically restricted ketogenic diets. Epilepsia 49 Suppl 8: 114-116).

As such, any conditions which restrict glucose availability (or impair glycolysis) while providing alternative energy sources for healthy cells, can selectively starve cancer cells while leaving normal cells unharmed. Metabolic therapy in the forms of dietary energy restriction or the ketogenic diet (KD) have been shown to elicit anti-cancer effects in a variety of cancers, likely by restricting glucose availability to the tumor and by inhibiting oncogenes that promote cancer progression (Seyfried & Shelton (2010) Cancer as a metabolic disease. Nutrition & metabolism 7: 7; Zhou, et al. (2007) The calorically restricted ketogenic diet, an effective alternative therapy for malignant brain cancer. Nutrition & metabolism 4: 5; Zuccoli, et al. (2010) Metabolic management of glioblastoma multiforme using standard therapy together with a restricted ketogenic diet: Case Report. Nutrition & metabolism 7: 33; Mavropoulos, et al. (2006) Is there a role for a low-carbohydrate ketogenic diet in the management of prostate cancer? Urology 68: 15-18). These metabolic strategies elevate blood ketone concentrations. Due to mitochondrial damage, most cancers are unable to utilize ketones for energy (Maurer, et al. (2011) Differential utilization of ketone bodies by neurons and glioma cell lines: a rationale for ketogenic diet as experimental glioma therapy. BMC Cancer. 2011 Jul. 26; 11:315; Skinner, et al. (2009) Ketone bodies inhibit the viability of human neuroblastoma cells. Journal of pediatric surgery 44: 212; Sawai, et al. (2004) Growth-inhibitory effects of the ketone body, monoacetoacetin, on human gastric cancer cells with succinyl-CoA: 3-oxoacid CoA-transferase (SCOT) deficiency. Anticancer research 24: 2213-2217; Tisdale & Brennan (1983) Loss of acetoacetate coenzyme A transferase activity in tumours of peripheral tissues. British journal of cancer 47: 293-297). Furthermore, ketones have been shown to inhibit cancer cell proliferation (Skinner, et al. (2009) Ketone bodies inhibit the viability of human neuroblastoma cells. Journal of pediatric surgery 44: 212; Sawai, et al. (2004) Growth-inhibitory effects of the ketone body, monoacetoacetin, on human gastric cancer cells with succinyl-CoA: 3-oxoacid CoA-transferase (SCOT) deficiency. Anticancer research 24: 2213-2217; Magee, et al. (1979) The inhibition of malignant cell growth by ketone bodies. The Australian journal of experimental biology and medical science 57: 529-539). For these reasons, we propose the use of supplemental ketone administration to enhance the efficacy of ketogenic diet metabolic therapy. Tumors also possess abnormal vasculature which blocks adequate tissue perfusion, leading to the presence of hypoxic regions that confer chemotherapy and radiation resistance and activate a number of oncogene pathways that promote cancer progression (Vaupel & Harrison (2004) Tumor hypoxia: causative factors, compensatory mechanisms, and cellular response. The oncologist 9 Suppl 5: 4-9; Hoogsteen, et al. (2007) The hypoxic tumour microenvironment, patient selection and hypoxia-modifying treatments. Clinical oncology (Royal College of Radiologists (Great Britain)) 19: 385-396; Vaupel, et al. (2001) Treatment resistance of solid tumors: role of hypoxia and anemia. Medical oncology (Northwood, London, England) 18: 243-259; Vaupel, et al. (2004) Tumor hypoxia and malignant progression. Methods in enzymology 381: 335-354). Hyperbaric oxygen therapy (HBO2T) increases oxygen concentration in tissues, potentially leading to a reversal of the cancer-promoting effects of tumor hypoxia (Vaupel & Harrison (2004) Tumor hypoxia: causative factors, compensatory mechanisms, and cellular response. The oncologist 9 Suppl 5: 4-9; Hoogsteen, et al. (2007) The hypoxic tumour microenvironment, patient selection and hypoxia-modifying treatments. Clinical oncology (Royal College of Radiologists (Great Britain)) 19: 385-396). Both metabolic therapy and HBO2T have been used to inhibit cancer progression and enhance the efficacy of radiation and chemotherapy in animal models; however, additional evidence is needed to determine the potential use of these non-toxic adjuvant treatments (Stuhr, et al. (2004) Hyperbaric oxygen alone or combined with 5-FU attenuates growth of DMBA-induced rat mammary tumors. Cancer letters 210: 35-75; Bennett, et al. (2008) Hyperbaric oxygenation for tumour sensitisation to radiotherapy: a systematic review of randomised controlled trials. Cancer treatment reviews 34: 577-591; Stafford, et al. (2010) The ketogenic diet reverses gene expression patterns and reduces reactive oxygen species levels when used as an adjuvant therapy for glioma. Nutrition & metabolism 7: 74; Takiguchi, et al. (2001) Use of 5-FU plus hyperbaric oxygen for treating malignant tumors: evaluation of antitumor effect and measurement of 5-FU in individual organs. Cancer chemotherapy and pharmacology 47: 11-14; Moen, et al. (2009) Hyperoxia increases the uptake of 5-fluorouracil in mammary tumors independently of changes in interstitial fluid pressure and tumor stroma. BMC cancer 9: 446).

In normal tissues, hypoxia inhibits mitochondrial production of ATP< stimulating an up-regulation of glycolysis to meet energy needs. Thus, the cellular response to tumor hypoxia is mediated by several of the same pathways that are overly active in cancer cells with mitochondrial damage and high rates of glycolysis. This suggests that metabolic therapy and HBO2T target several overlapping pathways and behaviors of cancer cells. Combining metabolic therapy with HBO2T will work synergistically to inhibit cancer progression. The addition of these non-toxic adjuvant therapies to the current standard of care could significantly improve the outcome of many patients with advanced metastatic disease.

While the major oncogene and tumor suppressor gene mutations can be found in many different cancers, one of the only universal traits of tumor cells across tissue types is abnormal energy metabolism (Seyfried & Shelton (2010) Cancer as a metabolic disease. Nutrition & metabolism 7: 7). In the 1930s, Otto Warburg observed that cancer cells express very high rates of aerobic glycolysis, or fermentation in the presence of oxygen (Warburg (1956) On the origin of cancer cells. Science 123: 309-314; Warburg (1956) On respiratory impairment in cancer cells. Science 124: 269-270). This feature, known as The Warburg Effect, is linked to mitochondrial dysfunction and genetic mutations within the cancer cell. While healthy cells derive the vast majority of their energy from ATP production by oxidative phosphorylation (OXPHOS) in the mitochondria, cancer cells rely almost exclusively on ATP production by substrate level phosphorylation (SLP) (Seyfried & Shelton (2010) Cancer as a metabolic disease. Nutrition & metabolism 7: 7). Nearly ubiquitously, cancers utilize SLP of glycolysis in the cytoplasm, as seen in FIG. 1, and, in some cancers, of glutaminolysis and the Kreb's Cycle (Lunt S Y, Vander Heiden M G (2011) Aerobic glycolysis: meeting the metabolic requirements of cell proliferation. Annual review of cell and developmental biology 27: 441-464; Medina (2001) Glutamine and cancer. The Journal of nutrition 131: 2539S-2542S; discussion 2550S-2531S). In fact, cancer cells undergo glycolysis at a rate up to 200-times that of healthy cells (Warburg (1956) On respiratory impairment in cancer cells. Science 124: 269-270). It is well documented that cancer cells across tissue types possess an array of mitochondrial damage, including loss of mitochondrial number, mitochondrial swelling, partial or total cristolysis, abnormalities in mitochondrial lipid composition, and absent, mutated, or decreased activity of mitochondrial enzymes involved in OXPHOS (Cuezva, et al. (2002) The bioenergetic signature of cancer: a marker of tumor progression. Cancer research 62: 6674-6681; Isidoro, et al. (2004) Alteration of the bioenergetic phenotype of mitochondria is a hallmark of breast, gastric, lung and oesophageal cancer. The Biochemical journal 378: 17-20; Arismendi-Morillo & Castellano-Ramirez (2008) Ultrastructural mitochondrial pathology in human astrocytic tumors: potentials implications pro-therapeutics strategies. Journal of electron microscopy 57: 33-42; Kiebish, et al. (2008) Cardiolipin and electron transport chain abnormalities in mouse brain tumor mitochondria: lipidomic evidence supporting the Warburg theory of cancer. Journal of lipid research 49: 2545-2601; Modica-Napolitano & Singh (2004) Mitochondrial dysfunction in cancer. Mitochondrion 4: 755-817; Kataoka, et al. (1991) Ultrastructural study of mitochondria in oncocytes. Ultrastructural pathology 15: 231-239). With this severe mitochondrial damage, cancer cells are unable to produce adequate amounts of ATP through OXPHOS to maintain viability and are forced to up-regulate SLP and glycolysis to survive (Seyfried & Shelton (2010) Cancer as a metabolic disease. Nutrition & metabolism 7: 7). Many of the genes that mediate this shift are known oncogenes and tumor suppressor genes. HIF-1α, IGF-1/PI3K/Akt, MYC, mTOR, and Ras upregulate glycolytic enzymes and GLUT transporter expression (Seyfried & Shelton (2010) Cancer as a metabolic disease. Nutrition & metabolism 7: 7; Miceli & Jazwinski (2005) Common and cell type-specific responses of human cells to mitochondrial dysfunction. Experimental Cell Research 302: 270-280); p53 and PTEN inhibit these responses and are thus inhibited (Liu & Feng (2012) PTEN, energy metabolism and tumor suppression. Acta biochimica et biophysica Sinica). This fermentative phenotype causes cancers to excrete large quantities of lactate, creating an acidic tumor microenvironment that promotes EMT and metastasis (Walenta, et al. (2000) High lactate levels predict likelihood of metastases, tumor recurrence, and restricted patient survival in human cervical cancers. Cancer research 60: 916-921; Dhup< et al. (2012) Multiple biological activities of lactic acid in cancer: influences on tumor growth, angiogenesis and metastasis. Current pharmaceutical design 18: 1319-1330). Lactate can also be returned to the cancer as glucose via the Cori Cycle, replenishing fuel for the glycolysis-dependent tumor cells, as seen in FIG. 1. Due to this metabolic deficiency, cancer cells have elevated rates of glucose consumption relative to healthy cells—a quality that underlies the use of fluorodeoxyglucose-PET scans as an important diagnostic tool for oncologists (Duranti, et al. (2012) PET scan contribution in chest tumor management: a systematic review for thoracic surgeons. Tumori 98: 175-184). Ketogenic diets (KDs) are high fat, low or no carbohydrate diets that have been used to treat pediatric refractory epilepsy for decades (Katyal, et al. (2000) The ketogenic diet in refractory epilepsy: the experience of Children's Hospital of Pittsburgh. Clinical pediatrics 39: 153-159). KDs naturally suppress appetite and often lead to dietary energy restriction (DER) and body weight loss (Katyal, et al. (2000) The ketogenic diet in refractory epilepsy: the experience of Children's Hospital of Pittsburgh. Clinical pediatrics 39: 153-159) or decreased lean body mass (Katyal, et al. (2000) The ketogenic diet in refractory epilepsy: the experience of Children's Hospital of Pittsburgh. Clinical pediatrics 39: 153-159; Paoli, et al. (2012) Ketogenic diet does not affect strength performance in elite artistic gymnasts. Journal of the International Society of Sports Nutrition 9: 34; Johnstone, et al. (2008) Effects of a high-protein ketogenic diet on hunger, appetite, and weight loss in obese men feeding ad libitum. The American journal of clinical nutrition 87: 44-55; Hussain, et al. (2012) Effect of low-calorie versus low-carbohydrate ketogenic diet in type 2 diabetes. Nutrition 28: 1016-1021; Volek, et al. (2004) Comparison of energy-restricted very low-carbohydrate and low-fat diets on weight loss and body composition in overweight men and women. Nutrition & metabolism 1: 13). While low carbohydrate or KDs promote weight loss in overweight individuals, they are known to spare muscle wasting during DER (Paoli, et al. (2012) Ketogenic diet does not affect strength performance in elite artistic gymnasts. Journal of the International Society of Sports Nutrition 9: 34; Manninen (2006) Very-low-carbohydrate diets and preservation of muscle mass. Nutrition & metabolism 3: 9; Cahill (2006) Fuel metabolism in starvation. Annual review of nutrition 26: 1-22; Veech (2004) The therapeutic implications of ketone bodies: the effects of ketone bodies in pathological conditions: ketosis, ketogenic diet, redox states, insulin resistance, and mitochondrial metabolism. Prostaglandins, leukotrienes, and essential fatty acids 70: 309-319). DER has been shown to slow disease progression in a variety of cancers, including brain, prostate, mammary, pancreas, lung, gastric, and colon (Seyfried & Shelton (2010) Cancer as a metabolic disease. Nutrition & metabolism 7: 7; Zuccoli, et al. (2010) Metabolic management of glioblastoma multiforme using standard therapy together with a restricted ketogenic diet: Case Report. Nutrition & metabolism 7: 33; Mavropoulos, et al. (2006) Is there a role for a low-carbohydrate ketogenic diet in the management of prostate cancer? Urology 68: 15-18; Zhou, et al. (2007) The calorically restricted ketogenic diet, an effective alternative therapy for malignant brain cancer. Nutrition & metabolism 4: 5; Mavropoulos, et al. (2009) The effects of varying dietary carbohydrate and fat content on survival in a murine LNCaP prostate cancer xenograft model. Cancer prevention research (Philadelphia, Pa.) 2: 557-565; Otto, et al. (2008) Growth of human gastric cancer cells in nude mice is delayed by a ketogenic diet supplemented with omega-3 fatty acids and medium-chain triglycerides. BMC cancer 8: 122; Masko, et al. (2010) Low-carbohydrate diets and prostate cancer: how low is “low enough”? Cancer prevention research (Philadelphia, Pa.) 3: 1124-1131; Tisdale & Brennan; A comparison of long-chain triglycerides and medium-chain triglycerides on weight loss and tumor size in a cachexia model.pdf; Wheatley, et al. (2008) Low-carbohydrate diet versus caloric restriction: effects on weight loss, hormones, and colon tumor growth in obese mice. Nutrition and cancer 60: 61-68; Rossifanelli, et al. (1991) Effect of Energy Substrate Manipulation on Tumor-Cell Proliferation in Parenterally Fed Cancer-Patients. Clinical Nutrition 10: 228-232). DER appears to facilitate its anti-cancer effects through several metabolic pathways, including inhibition of the IGF-1/PI3K/Akt signaling pathway which promotes proliferation and angiogenesis and inhibits apoptosis (Mukherjee, et al. (2002) Dietary restriction reduces angiogenesis and growth in an orthotopic mouse brain tumour model. British journal of cancer 86: 1615-1621; Mukherjee, et al. (1999) Energy intake and prostate tumor growth, angiogenesis, and vascular endothelial growth factor expression. Journal of the National Cancer Institute 91: 512-523; Thompson, et al. (2004) Effect of dietary energy restriction on vascular density during mammary carcinogenesis. Cancer research 64: 5643-5650; Hursting, et al. (2010) Calories and carcinogenesis: lessons learned from 30 years of calorie restriction research. Carcinogenesis 31: 83-89; Thompson, et al. (2003) Dietary energy restriction in breast cancer prevention. Journal of mammary gland biology and neoplasia 8: 133-142; Thompson, et al. (2004) Identification of the apoptosis activation cascade induced in mammary carcinomas by energy restriction. Cancer research 64: 1541-1545; Zhu, et al. (2005) Effects of dietary energy repletion and IGF-1 infusion on the inhibition of mammary carcinogenesis by dietary energy restriction. Molecular carcinogenesis 42: 170-176). DER has been shown to induce apoptosis in astrocytoma cells but protect normal brain cells from death through activation of adenosine monophosphate kinase (AMPK) (Mukherjee, et al. (2008) Differential effects of energy stress on AMPK phosphorylation and apoptosis in experimental brain tumor and normal brain. Molecular cancer 7: 37). The KD has been successfully used as an adjuvant therapy for Glioblastoma Multiforme (GBM) in a small number of case reports with patients exhibiting marked improvements in quality of life, dramatic slowing of tumor growth, or disappearance of tumor altogether (Zuccoli, et al. (2010) Metabolic management of glioblastoma multiforme using standard therapy together with a restricted ketogenic diet: Case Report. Nutrition & metabolism 7: 33; Nebeling & Lerner (1995) Implementing a ketogenic diet based on medium-chain triglyceride oil in pediatric patients with cancer. Journal of the American Dietetic Association 95: 693-697). Furthermore, a pilot trial of patients with advanced metastatic disease of varying tissue types reported that the KD improved emotional functioning and quality of life in terminally ill patients (Schmidt, et al. (2011) Effects of a ketogenic diet on the quality of life in 16 patients with advanced cancer: A pilot trial. Nutr Metab (Lond). 2011 Jul. 27; 8(1):54).

The KD lowers blood glucose levels, limiting the energy supply for cancer cells, while elevating circulating blood ketone concentration (Seyfried, et al. (2009) Targeting energy metabolism in brain cancer through calorie restriction and the ketogenic diet. Journal of cancer research and therapeutics 5 Suppl 1: 15; Klement & Kämmerer (2011) Is there a role for carbohydrate restriction in the treatment and prevention of cancer? Nutrition & metabolism 8: 75). The two most abundant and physiologically relevant ketone bodies are acetoacetate (ACA) and β-hydroxybutyrate (βHB). Ketone bodies are metabolized exclusively in the mitochondria via the Kreb's Cycle and OXPHOS coupled to the electron transport chain. Abundant literature reports that most if not all cancers possess severe mitochondrial dysfunction and enzyme deficiencies that prevent effective utilization of ketone bodies for energy (Maurer, et al. (2011) Differential utilization of ketone bodies by neurons and glioma cell lines: a rationale for ketogenic diet as experimental glioma therapy. BMC Cancer. 2011 Jul. 26; 11:315; Cuezva, et al. (2002) The bioenergetic signature of cancer: a marker of tumor progression. Cancer research 62: 6674-6681; Fearon, et al. (1988) Cancer cachexia: influence of systemic ketosis on substrate levels and nitrogen metabolism. The American journal of clinical nutrition 47: 42-48; Sawai, et al. (2004) Growth-inhibitory effects of the ketone body, monoacetoacetin, on human gastric cancer cells with succinyl-CoA: 3-oxoacid CoA-transferase (SCOT) deficiency. Anticancer research 24: 2213-2217; Seyfried, et al. (2003) Role of glucose and ketone bodies in the metabolic control of experimental brain cancer. British journal of cancer 89: 1375-1457; Oudard, et al. (1997) Gliomas are driven by glycolysis: putative roles of hexokinase, oxidative phosphorylation and mitochondrial ultrastructure. Anticancer research 17: 1903-1911; John (2001) Dysfunctional mitochondria, not oxygen insufficiency, cause cancer cells to produce inordinate amounts of lactic acid: the impact of this on the treatment of cancer. Medical hypotheses 57: 429-460; Wu, et al. (2007) Multiparameter metabolic analysis reveals a close link between attenuated mitochondrial bioenergetic function and enhanced glycolysis dependency in human tumor cells. American journal of physiology Cell physiology 292: C125-136). Many cancers do not express the Succinyl-CoA: 3-ketoacid CoA-Transferase (SCOT) enzyme, which is required for ketone body metabolism (Sawai, et al. (2004) Growth-inhibitory effects of the ketone body, monoacetoacetin, on human gastric cancer cells with succinyl-CoA: 3-oxoacid CoA-transferase (SCOT) deficiency. Anticancer research 24: 2213-2217; Tisdale & Brennan (1983) Loss of acetoacetate coenzyme A transferase activity in tumours of peripheral tissues. British journal of cancer 47: 293-297). βHB administration rescues healthy brain cells from glucose withdrawal-induced cell death but does not protect glioma cells (Maurer, et al. (2011) Differential utilization of ketone bodies by neurons and glioma cell lines: a rationale for ketogenic diet as experimental glioma therapy. BMC Cancer. 2011 Jul. 26; 11:315). It is well known that ketones can replace glucose to supply most of the brain's metabolic energy needs (>50%) during periods of limited glucose availability resulting from starvation, CR or carbohydrate restriction as in KD (Cahill 2006). Moreover, it is known that ketones are a more efficient mitochondrial energy source than glucose (reviewed in Veech, 2004). The invention described below causes a rapid and sustained elevation of blood ketones with a single oral administration. The therapeutic ketosis produced by the invention could reverse the metabolic dysregulation and oxidative stress associated with many neurological disorders.

While ketones are not an energy source for cancer cells, they are an efficient energy substrate for healthy tissue in the rest of the body. Ketones have been shown to inhibit cancer cell growth and proliferation in vitro in a variety of cell lines, including gastric cancer, transformed lymphoblasts, kidney cancer, HeLa cells, and melanoma (Magee, et al. (1979) The inhibition of malignant cell growth by ketone bodies. The Australian journal of experimental biology and medical science 57: 529-539; Sawai, et al. (2004) Growth-inhibitory effects of the ketone body, monoacetoacetin, on human gastric cancer cells with succinyl-CoA: 3-oxoacid CoA-transferase (SCOT) deficiency. Anticancer research 24: 2213-2217). It is unclear exactly how ketones elicit their anti-cancer effects. Ketone bodies are known to inhibit glycolysis, which may contribute to their efficacy (Wu & Thompson (1988) The effect of ketone bodies on alanine and glutamine metabolism in isolated skeletal muscle from the fasted chick. The Biochemical journal 255: 139-144). Additionally, ketones are transported into the cell via the monocarboxylate transporters (MCTs) which are also responsible for exporting the fermentation product lactate from the cell into the circulation. Lactate confers an acidic tumor microenvironment and is known to play a large role in invasion and metastasis (Dhup< et al. (2012) Multiple biological activities of lactic acid in cancer: influences on tumor growth, angiogenesis and metastasis. Current pharmaceutical design 18: 1319-1330). If ketone body uptake inhibits lactate export by competitive inhibition of the MCT transporters, this might be another mechanism by which ketones hinder cancer progression. Furthermore, it has been well-documented that both calorie restriction and fasting, conditions where ketones take over as a primary fuel, possess very potent anti-cancer effects, further supporting the observation that cancer cells cannot thrive by using ketone bodies for fuel (Hursting, et al. (2010) Calories and carcinogenesis: lessons learned from 30 years of calorie restriction research. Carcinogenesis 31: 83-89; Lee, et al. (2012) Starvation, detoxification, and multidrug resistance in cancer therapy. Drug resistance updates: reviews and commentaries in antimicrobial and anticancer chemotherapy 15: 114-122).

Since ketones appear to possess intrinsic anti-cancer effects, ketone supplementation to tissues is an interesting avenue for cancer therapy. Ketone esters (KE) are non-ionized, water-soluble precursors of ketone bodies that increase plasma ketone levels regardless of the status of dietary energy intake (Clarke, et al. (2012) Kinetics, safety and tolerability of (R)-3-hydroxybutyl(R)-3-hydroxybutyrate in healthy adult subjects. Regulatory toxicology and pharmacology: RTP 63: 401-408). When ingested, KEs elevate blood ketone levels proportionally to the amount of ester taken (Clarke, et al. (2012) Kinetics, safety and tolerability of (R)-3-hydroxybutyl(R)-3-hydroxybutyrate in healthy adult subjects. Regulatory toxicology and pharmacology: RTP 63: 401-408; Desrochers, et al. (1995) Metabolism of (R,S)-1,3-butanediol acetoacetate esters, potential parenteral and enteral nutrients in conscious pigs. The American journal of physiology 268: E660-667; Clarke, et al. (2012) Oral 28-day and developmental toxicity studies of (R)-3-hydroxybutyl(R)-3-hydroxybutyrate. Regulatory toxicology and pharmacology:RTP 63: 196-208). 1,3-butanediol (BD), an approved, non-toxic food additive and hypoglycemic agent, is a compound metabolized by the liver to produce βHB and can also be used as a source of supplemental ketones (Kies, et al. (1973) Utilization of 1,3-butanediol and nonspecific nitrogen in human adults. The Journal of nutrition 103: 1155-1163; td. C (2003) 1,3-Butanediol IUCLID Data Set). We propose that supplemental ketone administration with KE or BD will inhibit cancer progression as a stand-alone treatment and also enhance the efficacy of ketogenic diet therapy. As described, in vitro, in vivo, and human studies all indicate that metabolic therapy targeting the abnormal energy metabolism of cancer cells is a promising direction in cancer research.

Energy metabolism is closely tied to the oxygen saturation state of the cell. Since oxygen is a vital component of ATP production via OXPHOS in the mitochondria, a decrease in tissue oxygen availability (hypoxia) induces a shift towards ATP production via SLP and glycolysis. The two primary cellular mechanisms that respond to hypoxic stress are the AMP-Activated Protein Kinase (AMPK) and Hypoxia-Inducible Factor-1 (HIF-1) pathways. AMPK works as an energy sensor by measuring the AMP:ATP ratio of the cell, a symbol of the cellular energy status. Hypoxia decreases mitochondrial ATP production, promoting activation of the AMPK pathway which stimulates catabolic processes such as fatty acid oxidation and glycolysis to provide energy substrates for the cell (Laderoute, et al. (2006) 5′-AMP-activated protein kinase (AMPK) is induced by low-oxygen and glucose deprivation conditions found in solid-tumor microenvironments. Molecular and cellular biology 26: 5336-5347). AMPK also induces the translocation of glucose transporters (GLUT-4) to the cell membrane, enhancing glucose uptake in tissues (Russell, et al. (1999) Translocation of myocardial GLUT-4 and increased glucose uptake through activation of AMPK by AICAR. The American journal of physiology 277: H643-649; Li J, et al. (2004) Role of the nitric oxide pathway in AMPK-mediated glucose uptake and GLUT4 translocation in heart muscle. American journal of physiology Endocrinology and metabolism 287: E834-841). HIF-1α is the primary oxygen sensing mechanism in the tissue. At normal tissue PO₂, HIF-1α is degraded, and the HIF-1 transcription factor remains sequestered and inactive in the cytoplasm. When tissues become hypoxic, HIF-1α is stabilized, activating HIF-1 which translocates to the nucleus, acting as a transcription factor for several hypoxia-responsive genes. Since this mechanism evolved to promote survival during transient hypoxic conditions, it is not surprising that many of the genes under regulation by HIF-1 are known oncogenes, promoting growth, cell survival, angiogenesis, and inhibiting apoptosis (Wouters, et al. (2004) Targeting hypoxia tolerance in cancer. Drug resistance updates: reviews and commentaries in antimicrobial and anticancer chemotherapy 7: 25-40; Le Q-T, Denko N, Giaccia A (2004) Hypoxic gene expression and metastasis. Cancer metastasis reviews 23: 293-310).

Tumors possess abnormal vasculature which blocks adequate tissue perfusion, leading to the presence of large hypoxic regions with abnormally low tissue oxygen saturation (Vaupel, et al. (2001) Treatment resistance of solid tumors: role of hypoxia and anemia. Medical oncology (Northwood, London, England) 18: 243-259). Healthy tissue oxygen tension varies by tissue type, but tumors contain hypoxic pockets expressing markedly lower PO₂ compared to their tissue of origin (Hoogsteen, et al. (2007) The hypoxic tumour microenvironment, patient selection and hypoxia-modifying treatments. Clinical oncology (Royal College of Radiologists (Great Britain)) 19: 385-396). While the average healthy tissue PO₂ is 55 mmHg, tumors possess an average tissue PO₂ of 8 mmHg, with 25% of tumors exhibiting less than 2.5 mmHg (Hoogsteen, et al. (2007) The hypoxic tumour microenvironment, patient selection and hypoxia-modifying treatments. Clinical oncology (Royal College of Radiologists (Great Britain)) 19: 385-396; Gill & Bell (2004) Hyperbaric oxygen: its uses, mechanisms of action and outcomes. QJM 97). This severe hypoxia confers many growth advantages to the cancer, mostly through the actions of HIF-1 which activates several oncogene pathways that promote cancer progression and metastasis, as seen in FIG. 2 (Vaupel & Harrison (2004) Tumor hypoxia: causative factors, compensatory mechanisms, and cellular response. The oncologist 9 Suppl 5: 4-9; Vaupel, et al. (2004) Tumor hypoxia and malignant progression. Methods in enzymology 381: 335-354). HIF-1 can also be activated by lactate; therefore, it is often functioning throughout the tumor due to the fermentative phenotype of cancer cells (Dhup< et al. (2012) Multiple biological activities of lactic acid in cancer: influences on tumor growth, angiogenesis and metastasis. Current pharmaceutical design 18: 1319-1330). Furthermore, tumor hypoxia has been shown to contribute to chemotherapy and radiation resistance (Vaupel & Harrison (2004) Tumor hypoxia: causative factors, compensatory mechanisms, and cellular response. The oncologist 9 Suppl 5: 4-9; Hoogsteen, et al. (2007) The hypoxic tumour microenvironment, patient selection and hypoxia-modifying treatments. Clinical oncology (Royal College of Radiologists (Great Britain)) 19: 385-396; Vaupel, et al. (2001) Treatment resistance of solid tumors: role of hypoxia and anemia. Medical oncology (Northwood, London, England) 18: 243-259; Vaupel, et al. (2004) Tumor hypoxia and malignant progression. Methods in enzymology 381: 335-354). Hypoxic cancer cells are three-times more resistant to radiation therapy than well-oxygenated cells (Gray, et al. (1953) The concentration of oxygen dissolved in tissues at the time of irradiation as a factor in radiotherapy. The British journal of radiology 26: 638-648).

Hyperbaric oxygen therapy (HBO2T) is the administration of 100% oxygen at elevated pressure (greater than sea level, 1 ATA). HBO2T increases plasma oxygen saturation, facilitating oxygen delivery to the tissue independent of hemoglobin O₂ saturation (Gill & Bell (2004) Hyperbaric oxygen: its uses, mechanisms of action and outcomes. QJM 97). The potential benefit of using HBO2T to combat the cancer-promoting effects of tumor hypoxia is clear. HBO2T alone has been shown to inhibit tumor growth, reduce tumor blood vessel density, and induce the preferential expression of anti-cancer genes in rat models of mammary tumors (Raa, et al. (2007) Hyperoxia retards growth and induces apoptosis and loss of glands and blood vessels in DMBA-induced rat mammary tumors. BMC cancer 7: 23; Stuhr, et al. (2007) Hyperoxia retards growth and induces apoptosis, changes in vascular density and gene expression in transplanted gliomas in nude rats. Journal of neuro-oncology 85: 191-393). Additionally, radiation and many chemotherapy drugs work by producing free radicals within the tumors, leading to cell death. Cancer cells with mitochondrial damage and chaotic oxygen perfusion produce chronically elevated levels of reactive oxygen species (ROS) but are susceptible to oxidative damage-induced cell death with even modest increases in ROS (Daruwalla & Christophi (2006) Hyperbaric oxygen therapy for malignancy: a review. World journal of surgery 30: 2112-2143; Aykin-Burns, et al. (2009) Increased levels of superoxide and H₂O₂ mediate the differential susceptibility of cancer cells versus normal cells to glucose deprivation. The Biochemical journal 418: 29-66; Schumacker (2006) Reactive oxygen species in cancer cells: live by the sword, die by the sword. Cancer cell 10: 175-181). HBO2T enhances tumor-cell production of ROS which can damage or kill cancer cells (D'Agostino, et al. (2009) Acute hyperoxia increases lipid peroxidation and induces plasma membrane blebbing in human U87 glioblastoma cells. Neuroscience 159: 1011-1033) and likely contributes to the synergistic effects of HBO2T as an adjuvant treatment to standard care (Schumacker (2006) Reactive oxygen species in cancer cells: live by the sword, die by the sword. Cancer cell 10: 175-181). Indeed, HBO2T has been demonstrated to enhance the efficacy of both radiation and chemotherapy in animal models (Stuhr, et al. (2004) Hyperbaric oxygen alone or combined with 5-FU attenuates growth of DMBA-induced rat mammary tumors. Cancer letters 210: 35-75; Bennett, et al. (2008) Hyperbaric oxygenation for tumour sensitisation to radiotherapy: a systematic review of randomised controlled trials. Cancer treatment reviews 34: 577-591; Takiguchi, et al. (2001) Use of 5-FU plus hyperbaric oxygen for treating malignant tumors: evaluation of antitumor effect and measurement of 5-FU in individual organs. Cancer chemotherapy and pharmacology 47: 11-14; Daruwalla & Christophi (2006) Hyperbaric oxygen therapy for malignancy: a review. World journal of surgery 30: 2112-2143; Moen, et al. (2009) Hyperoxic treatment induces mesenchymal-to-epithelial transition in a rat adenocarcinoma model. PloS one 4; Petre, et al. (2003) Hyperbaric oxygen as a chemotherapy adjuvant in the treatment of metastatic lung tumors in a rat model. The Journal of thoracic and cardiovascular surgery 125: 85; Moen & Stuhr (2012) Hyperbaric oxygen therapy and cancer—a review. Targeted oncology). Preclinical data suggests HBO2T is efficacious in the treatment of cancer, but additional studies are needed to support its use (Moen & Stuhr (2012) Hyperbaric oxygen therapy and cancer—a review. Targeted oncology).

The mechanisms linking hypoxia to energy metabolism are numerous and have been expertly described in recent reviews (Ralph, et al. (2010) The causes of cancer revisited: “mitochondrial malignancy” and ROS-induced oncogenic transformation—why mitochondria are targets for cancer therapy. Molecular aspects of medicine 31: 145-170; Fogg, et al. (2011) Mitochondria in cancer: at the crossroads of life and death. Chinese journal of cancer 30: 526-539; Taylor (2008) Mitochondria and cellular oxygen sensing in the HIF pathway. The Biochemical journal 409: 19-26). In normal tissues, decreased oxygen availability inhibits OXPHOS and mitochondrial production of ATP< stimulating an up-regulation of glycolytic enzymes to meet energy needs by SLP. Thus, the cellular response to tumor hypoxia is mediated by several of the same pathways that are overly active in cancer cells with mitochondrial damage and high rates of aerobic glycolysis. This suggests that KD or ketone supplement metabolic therapy and HBO2T target several overlapping pathways and tumorigenic behaviors of cancer cells. Importantly, metastatic cancer cells are notoriously glycolytic and hypoxic (Dhup< et al. (2012) Multiple biological activities of lactic acid in cancer: influences on tumor growth, angiogenesis and metastasis. Current pharmaceutical design 18: 1319-1330; Jiang, et al. (2011) EMT: a new vision of hypoxia promoting cancer progression. Cancer biology & therapy 11: 714-723; Lee & Simon (2012) From stem cells to cancer stem cells: HIF takes the stage. Current opinion in cell biology 24: 232-235; Hill, et al. (2009) Cancer stem cells, hypoxia and metastasis. Seminars in radiation oncology 19: 106-111), suggesting that targeting these phenotypes may be effective methods of inhibiting or treating metastatic disease. Combining these treatments will work synergistically to inhibit cancer progression in a preclinical model of systemic metastatic cancer. Further, as the present invention targets a metabolic phenotype that is present in most cancers regardless of the tissue of origin, i.e. the Warburg Effect, it is effective against any glycolytic cancer. The most common diagnostic tool that oncologists use is the FDG-PET scan, which scans for enhanced glucose uptake using the metabolic phenotype targeted by the invention, to diagnose nearly all types of cancers. Therefore, any cancer which utilizes this pathway, which is most, if not all cancers, will be treatable using the present invention.

The use of these metabolic strategies as stand-alone treatments or their addition as adjuvant therapies to standard of care may significantly improve the outcome of many patients with advanced metastatic disease.

### Cellular Effects of CNS-OT

Hyperbaric oxygen-induced seizures, also known as central nervous system oxygen toxicity (CNS-OT) compromise the safety of undersea divers and patients undergoing HBO₂ therapy (HBOT) (Clark and Thom 1997). This condition manifests as tonic-clonic seizures, which carry a significant risk of drowning for divers. Breathing 100% O₂ at P_{B}>2.4 ATA increases the likelihood of seizures in patients, and current applications of HBOT routinely use up to 3 ATA HBO₂ (Tibbles and Edelsberg 1996). The potential for CNS-OT is the primary limiting factor in HBOT. CNS-OT occurs with little or no warning and no effective mitigation strategy against it has been identified. Since HBO₂ provides a unique, reversible and reproducible stimulus for generalized tonic-clonic seizures in animal models, it is an effective model for assessing the neuroprotective potential of anticonvulsant strategies for epilepsy.

The free radical theory of O₂ toxicity predicts the body's antioxidant defenses are overwhelmed by increased production of reactive oxygen species (ROS) (Gerschman, 1954). This theory is supported by the observation that brain levels of ROS and reactive nitrogen species (RNS) increase just prior to HBO₂-induced seizures (Demchenko et al. 2003). Other investigators have confirmed ROS is elevated in various brain regions (Piantadosi and Tatro 1990) and in the blood during hyperoxia (Narkowicz et al. 1993).

It was shown that caudal solitary complex (SC) neurons and CA1 hippocampal neurons in brain slices are strongly stimulated by pro-oxidants and HBO₂ via redox signaling (Dean et al. 2003). In addition, superoxide production and neuronal excitability in the CA1 hippocampus is tightly coupled to tissue O₂ concentration ranging from 20-95% (D'Agostino et al. 2007). Using Ethidium Homodimer-1 (EH-1) staining in hippocampal slices, the inventors have shown an 02-dependent increase in cell death of CA1 neurons, with the highest level of cell death observed after 4 hr exposure to 95% O₂ (D'Agostino et al. 2007). Evidence suggests that hyperoxia-induced cell death is correlated to mitochondrial function impairment (Li et al. 2004a; Metrailler-Ruchonnet et al. 2007). More specifically, the mitochondrial-dependent cell death involves mitogen-activated protein kinase, proapoptotic Bcl-2 and ultimately mitochondrial depolarization and membrane depolarization (Chandel and Budinger 2007).

Considering the cellular and physiological effects of CNS-OT and the neuroprotective effect of therapeutic ketosis, the inventors induced ketosis as a metabolic strategy to prevent CNS-OT. Ketones may counteract the effects of CNS-OT by a variety of mechanisms, including 1) decreasing ROS production (Kim do et al. 2010); 2) enhancing mitochondrial efficiency (Veech 2004); 3) and acting as a direct anticonvulsant (Gasior et al. 2007; Likhodii et al. 2008).

Previous studies in rats show that starvation delays the onset of CNS-OT (Bitterman et al. 1997), presumably by fundamentally shifting brain energy metabolism. Starvation (24-36 h) also delays the latency to seizure from HBO₂ by up to 300%, which is equally or more effective than high doses of anti-epileptic drugs (AEDs) (Bitterman and Katz 1987; Tzuk-Shina et al. 1991) or than experimental anticonvulsants that block excitatory glutamatergic neurotransmission (Chavko et al. 1998).

During periods of starvation or ketogenic diet (KD) use, the body utilizes energy obtained from free fatty acids (FFA) released from adipose tissue; however, the brain is unable to derive significant energy from FFA (Cahill 2006). Hepatic ketogenesis converts FFAs into the ketone bodies β-hydroxybutyrate (BHB) and acetoacetate (AcAc), and a small percentage of AcAc spontaneously decarboxylates to acetone. During prolonged starvation or KD, large quantities of ketone bodies accumulate in the blood (>5 mM) and are transported across the blood brain barrier (BBB) by monocarboxylic acid transporters (MCT1-4) to fuel brain function, and this ketone transport is enhanced under oxidative stress or limited glucose availability (Prins 2008). The brain derives up to 75% of its energy from ketones when glucose availability is limited (Cahill 2006). Starvation and dietary ketosis are often confused with diabetic ketoacidosis (DKA), but this occurs only in the absence of insulin (VanItallie and Nufert 2003). At least two feedback loops prevent runaway ketoacidosis from occurring, including a ketone-induced release of insulin and ketonuria (Cahill 2006). The metabolic adaptations associated with starvation-induced ketosis improve mitochondrial function, decrease reactive oxygen species (ROS) production, reduce inflammation and increase the activity of neurotrophic factors (Maalouf et al. 2009).

KD mimics the metabolic state associated with starvation (i.e. therapeutic ketosis) and is efficacious in treating drug-resistant seizure disorders (Freeman and Kossoff 2010). This therapeutic method is well established in children and adults (Klein et al. 2010). The anticonvulsant effects of the KD correlate with an elevation of blood ketones, especially AcAc and acetone (Bough and Rho 2007; McNally and Hartman 2011). The KD requires extreme dietary carbohydrate restriction and only modestly increases blood ketones compared to levels associated with prolonged starvation (Cahill 2006). In addition, the unbalanced macronutrient profile of the KD is often considered unpalatable and has the potential to negatively impact lipid profile if consumed in unrestricted amounts (Freeman and Kossoff 2010).

Elevating blood ketones with ketogenic medical foods or exogenous ketones is largely ineffective or problematic for a variety of reasons. Ketogenic fats, like medium chain triglyceride oil (MCT oil) are generally not well tolerated by the gastrointestinal system, and supplementation produces only low levels of ketones (<0.5 mM) (Henderson 2008). Oral administration of BHB and AcAc in their free acid form is expensive and ineffective at producing sustained ketosis. One idea has been to buffer the free acid form of BHB with sodium salts, but this is largely ineffective at preventing seizures in animal models and causes a potentially harmful sodium overload at therapeutic levels of ketosis (Bough and Rho 2007). However, esters of BHB or AcAc can effectively induce a rapid and sustained ketosis (Brunengraber 1997; Desrochers et al. 1995) that mimics the sustained ketosis achieved with a strict KD or prolonged starvation without dietary restriction. Producing esters of BHB or AcAc is expensive and technically challenging, but offers great therapeutic potential (Veech 2004). Orally administered KEs have the potential to induce ketosis and circumvent the problems associated with starvation-induced or diet-induced ketosis.

The KE that the inventors have synthesized and tested, R,S-1,3-butanediol acetoacetate diester (BD-AcAc₂), has been shown to induce therapeutic ketosis in dogs (Ciraolo et al. 1995; Puchowicz et al. 2000) and pigs (Desrochers et al. 1995) and was proposed as a metabolic therapy for parenteral and enteral nutrition (Brunengraber 1997). The inventors were interested in esters of AcAc because precursors to BHB do not prevent CNS-OT (Chavko et al. 1999), and animal studies suggest that AcAc and acetone have the greatest anticonvulsant potential (Bough and Rho 2007; Gasior et al. 2007; Likhodii et al. 2003; McNally and Hartman 2011).

The anticonvulsant mechanism the KD is largely unknown (Bough and Rho 2007). Proposed mechanisms for the anticonvulsant effect include, but are not limited to, decreased blood glucose, increased inhibitory neuromodulators, diminished excitatory neurotransmission and enhanced mitochondrial function by ketones (Greene et al. 2003; Hartman et al. 2007; Jahn 2010; Masino et al. 2009). The anticonvulsant mechanism of the KD is of great importance for those involved in developing anti-seizure therapies. There exists an intense interest to develop a substance that produces a rapid, safe and sustained elevation of blood ketones for prevention of seizures, a “ketogenic diet in a pill” (Rho and Sankar 2008). Ketone administration (independent from the KD) may directly mediate anticonvulsant effects by virtue of acetoacetate (AcAc) decarboxylating to acetone, a lipophilic solvent with strong anticonvulsant effects (Bough and Rho 2007; Likhodii et al. 2008). In addition, ketones may prevent synaptic dysfunction by preserving mitochondrial metabolism, reducing ROS (Kim do et al. 2010) and supplying an alternative form of energy with a higher AG′ value of ATP hydrolysis (Veech 2004).

Evidence for the KD working through novel ketone-induced mechanisms is supported by the fact that the KD works when even high doses of multiple antiepileptic drugs (AEDs) fail (Kim do and Rho 2008). Thus, the KD activates mechanisms other than those targeted by any specific AED, or even combinations of AEDs. Surprisingly, no commercially available AEDs attempt to mimic therapeutic ketosis conferred by the KD. However, evidence suggests that a common ketogenic precursor (MCT oil) induces a very mild ketosis that confers anticonvulsant effects (Neal et al. 2009) and improves mild cognitive impairment in patients by (Henderson 2008). Interestingly, inducing ketosis by administration of the primary ketone, beta-hydroxybutyrate (BHB), or BHB precursors does not prevent acutely provoked seizures in animal models (Bough and Rho 2007) including CNS-OT (Chavko et al. 1999). In contrast, elevation of Acc and acetone prevents acutely provoked seizures (chemical, electrical) in animal models (Likhodii et al. 2008; Rho et al. 2002; Yamashita 1976) including CNS-OT (Chavko et al. 1999). Acetone is relatively nontoxic (LD50 >5 g/kg; rat) and anticonvulsant at subnarcotic concentrations (Gasior et al. 2007; Likhodii et al. 2003) and its anticonvulsant effect is due to its membrane stabilizing lipophilic properties. Taken together, these observations suggest that methods of therapeutic ketosis for treatment of CNS O₂ toxicity and seizures should be designed to elevate AcAc, which is typically in a 1:4 ratio with BHB.

### SUMMARY OF THE INVENTION

Central nervous system oxygen toxicity (CNS-OT) seizures occur with little or no warning, and no effective mitigation strategy has been identified. Ketogenic diets (KD) elevate blood ketones and have successfully treated drug-resistant epilepsy. The inventors administered a ketone ester (KE) orally as a non-ionized precursor of acetoacetate (AcAc), R,S-1,3-butanediol acetoacetate diester (BD-AcAc₂) to delay seizures in rats breathing hyperbaric oxygen (HBO₂) at 5 atmospheres absolute (ATA). KE was found to cause a rapid and sustained (>4 h) elevation of BHB (>3 mM) and AcAc (>3 mM), which exceeded values reported with a KD or starvation. KE increased the latency to seizure (LS) by 574±116% compared to control (water), and was due to the effect of AcAc and acetone, but not BHB. BD produced ketosis in rats by elevating BHB (>5 mM), but AcAc and acetone remained low or undetectable. BD did not increase LS. It was found that acute oral administration of KE produced sustained therapeutic ketosis and significantly delayed CNS-OT by elevating AcAc and acetone. KE represents a novel therapeutic mitigation strategy for CNS-OT and seizure disorders, especially AED-resistant seizures.

Accordingly, a method is presented for treating metabolic dysregulation, such as Alzheimer's disease, central nervous system oxygen toxicity, seizures, or cancer. The method includes administering to an animal a ketogenic diet, followed by subjecting the animal to a hyperbaric, oxygen-enriched environment. The hyperbaric, oxygen-enriched environment is optionally 100% oxygen, and can be 2.5 absolute atmosphere. In some embodiments, the animal is subjected to the hyperbaric, oxygen-enriched environment for 90 minutes three times a week.

Additionally, the treatment is optionally supplemented with ketones, such as acetoacetate, adenosine monophosphate kinase, 1,3-butanediol, ketone ester, 1,3-butanediol acetoacetate monoester, 1,3-butanediol acetoacetate diester, MCT oil, or R,S-1,3-butanediol-diacetoacetate ester. The ketone ester supplement is optionally administered at a dose of 10 g/kg. An exemplary ketone supplement includes a combination of R,S-1,3-butanediol acetoacetate monoester and R,S-1,3-butanediol acetoacetate diester. The supplementation may be at 10% to 20%, such as 10%, 11%, 12%, 13%, 14%, 15%, 16%, 17%, 18%, 19%, or 20%. Further, the ketone ester is administered about 30 minutes prior to subjecting the animal to the hyperbaric, oxygen-enriched environment.

A method of protecting against central nervous system oxygen toxicity, convulsions, or hyperoxia-induced oxidative stress is also provided herein. The method includes administering a therapeutically effective dose of a acetoacetate, adenosine monophosphate kinase, 1,3-butanediol, ketone ester, 1,3-butanediol acetoacetate monoester, 1,3-butanediol acetoacetate diester, MCT oil, or R,S-1,3-butanediol-diacetoacetate ester at a predetermined time period, administering to an animal a ketogenic diet. and subjecting the animal to a hyperbaric, oxygen-enriched environment. The acetoacetate, adenosine monophosphate kinase, 1,3-butanediol, ketone ester, 1,3-butanediol acetoacetate monoester, 1,3-butanediol acetoacetate diester, MCT oil, or R,S-1,3-butanediol-diacetoacetate ester is optionally administered about 30 minutes prior to subjecting the animal to the hyperbaric, oxygen-enriched environment. Further, the acetoacetate, adenosine monophosphate kinase, 1,3-butanediol, ketone ester, 1,3-butanediol acetoacetate monoester, 1,3-butanediol acetoacetate diester, MCT oil, or R,S-1,3-butanediol-diacetoacetate ester is optionally administered at between 10% to 20% of the ketogenic diet, such as 10%, 11%, 12%, 13%, 14%, 15%, 16%, 17%, 18%, 19%, or 20%, or at 10 g/kg

The hyperbaric, oxygen-enriched environment is optionally 100% oxygen, and can be 2.5 absolute atmosphere. In some embodiments, the animal is subjected to the hyperbaric, oxygen-enriched environment for 90 minutes three times a week.

### BRIEF DESCRIPTION OF THE DRAWINGS

For a fuller understanding of the invention, reference should be made to the following detailed description, taken in connection with the accompanying drawings, in which:

FIG. 1 is an illustration of energy metabolism of cancer cell compared to a normal cell.

FIG. 2 is an illustration showing linking hypoxia to cancer progression. Image from Vaupel, P. et. al (Vaupel & Harrison (2004) Tumor hypoxia: causative factors, compensatory mechanisms, and cellular response. The oncologist 9 Suppl 5: 4-9).

FIG. 3 is an image depicting that acetone readily crosses the BBB whereas acetoacetate and B-hydroxybutyrate are transported via the monocarboxylic acid transporter (MCT). (Hartman et al. Pediatric Neurology. 2007 May; 36(5): 281-292)

FIGS. 4(A)-(C) are a series of images depicting the effect of ketones superoxide production (dihydroethidium fluorescence; DHE) in neurons treated with Aβ42 and HBO and on cell viability of U87MG cells (cancer cells). (A) Superoxide anion production was significantly lower in ketone treated cells under normobaric pressure (NBO) and hyperbaric pressure (HBO); (B) In case of Aβ42 treated cells a significant reduction of ROS production was observed in NBO and HBO groups treated with ketones. (n=12 cultures/group; *, P<0.05). (C) The total number of dead (ethidium homodimer-1) U87 cells was similar between groups, but the percentage of live (calcein) cancer cells significantly decreased in ketone-treated (2 mM ketones) cultures. (n=30 culture dishes/group; *, P<0.05).

FIGS. 5(A)-(P) are images depicting superoxide production (DHE fluorescence) in the CA1 region of a hippocampal brain slice preparation exposed to graded levels of oxygen over 4 hours. (A) cells treated with 95% oxygen and stained for superoxide production at 0 hr; (B) cells treated with 60% oxygen and stained for superoxide production at 0 hr; (C) cells treated with 40% oxygen and stained for superoxide production at 0 hr; (D) cells treated with 20% oxygen and stained for superoxide production at 0 hr; (E) cells treated with 95% oxygen and stained for superoxide production at 0 hr; (F) cells treated with 60% oxygen and stained for superoxide production at 4 hr; (G) cells treated with 40% oxygen and stained for superoxide production at 4 hr; (H) cells treated with 20% oxygen and stained for superoxide production at 4 hr; (I) cells treated with 95% oxygen and stained for cell death at 0 hr; (J) cells treated with 60% oxygen and stained for cell death at 0 hr; (K) cells treated with 40% oxygen and stained for cell death at 0 hr; (L) cells treated with 20% oxygen and stained for cell death at 0 hr; (M) cells treated with 95% oxygen and stained for cell death at 4 hr; (N) cells treated with 60% oxygen and stained for cell death at 4 hr; (O) cells treated with 40% oxygen and stained for cell death at 4 hr; (P) cells treated with 20% oxygen and stained for cell death at 4 hr. Note the oxygen-dependent increase in superoxide production. Hyperoxia-induced superoxide production was associated with increased cell death (ethidium homodimer-1 staining) (D'Agostino et. al)

FIG. 6 is an image depicting the effect of ketones (2 mM ketones) and a sigma receptor agonist, 1,3,-di-o-tolylguanidine (DTG), on superoxide anion production (DHE fluorescence) in primary cultures of rat cortical neurons under control conditions and hyperbaric oxygen (5 ATA O₂). Primary cortex neurons grown for 10 days under normal conditions were exposed to acute hyperoxia (60 min, 5 ATA O₂). HBO₂ caused a significant increase in superoxide anion production in cells. Ketone treatment decreased baseline superoxide production in a way that resembled the effect of the neuroprotective drug DTG. Both ketones and DTG prevented the hyperoxia-induced increase in superoxide production (n=110 cells analyzed/condition, * indicates p≦0.005).

FIG. 7 is an image depicting the effect of ketones (2 mM ketones) on superoxide anion production in primary cortex neurons exposed to 1 mM of amyloid beta peptides (Aβ40, Aβ42), the peptide associated with Alzheimer's disease pathology. Ketones prevented excess ROS production associated with toxic levels of Ab.

FIG. 8 is an image depicting the blood levels of ketones following oral administration of ketone ester. Specifically, the mean blood β-hydroxybutyrate (βHB) level is shown 2-3 hours after oral administration of R,S-1,3 butanediol acetoacetate monoester (BD-AcAc).

FIG. 9 is an image depicting an electroencephalogram (EEG) signal, showing the latency time to seizure during hyperbaric hyperoxia (HBO₂) at 60 pounds per square inch (PSI) (5 ATA O₂). EEG recordings are a measurement of brain seizure activity. Seizure occurred in 8 minutes without ketone ester administered.

FIG. 10 is an image depicting an electroencephalogram (EEG) signal, showing the latency time to seizure during hyperbaric hyperoxia (HBO₂) at 60 pounds per square inch (PSI) (5 ATA O₂) following administration of KE (BD-AcAc).. EEG recordings are a measurement of brain seizure activity. Seizure was delayed for 110 minutes.

FIG. 11 is a graph depicting the resistance to CNS oxygen toxicity (5 ATA O₂). The responses of individual rats with no treatment, control (water) and administration of ketone ester (R,S-1,3 butanediol acetoacetate monoester) are shown. As shown in the graph, intragastric administration of KE (BD-AcAc) protects rats against CNS oxygen toxicity. Administration of ketone ester (3 ml gavage) 30 minutes prior to hyperbaric oxygen (5 ATA O₂) exposure significantly increased latency time to first electrical discharge (FED) of EEG.

FIG. 12 is a graph depicting the time to oxygen toxicity. The responses of rats without treatment, control (water) and administration of KE (BD-AcAc) are compared.

FIG. 13 is a graph depicting the effect of ketone esters on latency to seizure in rats exposed to 5 ATA O₂. As shown in the graph, acute intragastric administration of ketone esters (10 g/kg), a non-ionized precursor to ketone bodies, given 30 min before diving, delayed seizures in rats exposed to 5 ATA O₂.

FIGS. 14(A)-(D) are a series of images depicting examples of EEG raw data acquisition after the administration of (A) water (n=38), (B) BD (n=6) and (C) KE (n=16). (D) Percent change in LS relative to control: Oral administration of KE caused a significant increase in LS at 5 ATA O₂.

FIG. 15 is a graph depicting ketone diester causes a rapid and sustained increase in total blood plasma ketones.

FIG. 16 is a graph depicting blood plasma levels of BHB in rats (n=6 rats/group) semi-fasted (18 hrs) and gavaged with 3 mL (˜10 g/kg) of water (control), R,S-1,3-Butanediol acetoacetate diester (BD-AcAc₂) (KE) or R,S-1,3-Butanediol (BD). As shown in the graph BHB level was elevated compared to control after administration of either ketogenic compound.

FIG. 17 is a graph depicting blood plasma levels of AcAc in rats (n=6 rats/group) semi-fasted (18 hrs) and gavaged with 3 mL (˜10 g/kg) of water (control), BD-AcAc₂ (KE) or R,S-1,3-Butanediol (BD). As shown in the graph, AcAc level was increased significantly by the ketone ester as compared to water or BD.

FIG. 18 is a graph depicting the change in blood glucose in all groups in response to BD-AcAc₂, which represents a calorically dense (>6 kcal/gram) substance that does not elevate blood glucose. As shown in the graph, blood glucose did not change significantly in any group.

FIG. 19 is a graph depicting a subject's blood levels of BHB in response to BD-AcAc₂ (KE), 1,3-butanediol (1,3-BD) and ketogenic diet (KD) supplemented with MCT oil.

FIGS. 20(A)-(D) are a series of graphs depicting blood ketones and glucose levels following administration of water, KE and BD. (A) (similar to FIG. 16): BHB level was elevated compared to control after administration of either ketogenic compounds; (B) (similar to FIG. 17): AcAc level was increased significantly more by KE compared to water or BD; (C): acetone level increased significantly more after treatment with KE and (D) (similar to FIG. 18): blood glucose level did not change significantly in any group. n=6 rats/group; (NS=not significant).

FIG. 21 is a graph depicting blood gas values and pH following administration of water, KE and BD. (similar to FIG. 22): pO₂ was elevated after administration of KE. n=6 rats/group.

FIG. 22 is an image depicting to BD-AcAc₂ improves oxygenation in the blood as shown by pO₂ being elevated after administration of the ketone ester.

FIG. 23 is a graph depicting pCO₂ is elevated after administration of BD which may indicate that suppression of CNS function due to intoxication from the di-alcohol is a potential problem with raising blood ketones with BD.

FIG. 24 is a graph depicting blood gas values and pH following administration of water, KE and BD. (similar to FIG. 23): pCO₂ was elevated after administration of BD; n=6 rats/group.

FIG. 25 is a graph depicting increasing blood ketones with BD and BD-AcAc₂ causes a mild nonpathological acidosis (from 7.45 to 7.35).

FIG. 26 is a graph depicting blood gas values and pH following administration of water, KE and BD. (similar to FIG. 25): pH was elevated compared to control after administration of either KE or BD; n=6 rats/group.

FIG. 27 is a graph showing animals in the treatment groups showed slower tumor growth than controls. Total body bioluminescence was measured weekly as a measure of tumor size; error bars represent ±SEM.

FIGS. 28(A) and (B) are graphs showing βHB decreases VM-M3 cell proliferation and viability in vitro. (A) VM-M3 proliferation was inhibited when grown in control media supplemented with 5 mM βHB. Cell density was significantly less in ketone supplemented cells at 24, 48, 72 and 96 hours compared to controls (**p<0.01, ***p<0.001; One-Way ANOVA). (B) VM-M3 viability was decreased when grown in the presence of 5 mM βHB. There was a significantly smaller percentage of live cells in βHB treated versus control media (***p<0.001; Two-tailed student's t-test). Results were considered significant when p<0.05. Error bars represent ±SEM.

FIG. 29 is a Kaplan-Meier survival plot graph of study groups showing supplemental ketone administration increases survival time in mice with systemic metastatic cancer. All treatment groups exhibited a significantly different survival from control animals by the Logrank Survival Test (p=0.05) and significant increases in mean survival time means compared to control animals by two-tailed student's t-test (p<0.05.).

FIG. 30 is a graph showing supplemental ketones extend survival time in mice with systemic metastatic cancer. Kaplan-Meier survival curve of treatment groups. BD and KE, but not CR, treated mice demonstrated prolonged survival compared to controls (Logrank test for survival distribution; *p=0.02, **p=0.01, and p=0.37 respectively).

FIG. 31 is a graph showing animal weight. The graph bars indicate average percent of initial body weight for animals at weeks 2, 4, and 6. Error bars represent ±SEM.

FIG. 32 is a graph showing blood glucose levels in animals. Mice receiving supplemental ketone ester (SDKE and KDKE) showed significantly lower glucose than controls on day 7 (p<0.01). Animals in the SDKE, KDKE, and KDBD groups had significantly lower blood glucose levels than controls on day 14 (p<0.01).

FIG. 33(A)-(B) are images showing the effect of supplemental ketones on tumor bioluminescence. Ketone supplement fed mice demonstrated a trend of slower tumor growth compared to controls. (A) Bioluminescence tracked over time as a measure of tumor growth rate. Treated animals exhibited a trend of slower tumor growth rate compared to controls, although bioluminescence was not significantly different from controls at week 3. Error bars represent ±SEM. (B) Representative animals from each group at 21 days post tumor cell inoculation showing whole body tumor bioluminescence. Treated mice exhibited reduced metastatic spread. The control has a ROI of 2=6.457×10⁶; CR has a ROI of 2=1.700×10⁶; BD has a ROI of 2=1.739×10⁶; KE has a ROI of 3=1.536×10⁶.

FIG. 34(A)-(C) are graphs showing effect of ketone supplementation on blood glucose, blood βHB, urine AcAc, and body weight in mice. (A-C) blood glucose; (B) blood levels of ketones; and (C) urine AcAc of healthy VM/Dk mice 0-12 hours after feeding following 8 hour fast. BD and KE treated mice demonstrated decreased blood glucose and elevated blood βHB following feeding (*p<0.05; **p<0.01; ***p<0.001; Two-Way ANOVA). KE, but not BD, treated mice demonstrated elevated urine AcAc 12 hours after feeding (***p<0.001; One-Way ANOVA). Error bars represent ±SEM.

FIG. 35 is a graph showing β-hydroxybutyrate levels in animals. KDKE and KDBD groups had significantly higher blood ketones than controls on both day 7 and day 14 (p<0.05).

FIGS. 36(A)-(C) are graphs showing effect of ketone supplementation on blood (A) Blood glucose, (B) ketones, and (C) body weight of survival study VM-M3 mice. (A) CR and KE treated mice had lower glucose than controls by day 7 (**p<0.01; ***p<0.001; One-Way ANOVA). (B) CR and KE mice had elevated blood βHB compared to controls at day 7 (*p<0.05; ***p<0.001; One-Way ANOVA). (C) CR and KE mice had a 20% reduction in body weight compared to controls at day 14 which was sustained for the duration of study (***p<0.001; One-Way ANOVA). Results were considered significant when p<0.05. Error bars represent ±SEM.

FIGS. 37(A)-(B) are graphs showing (A) decreased blood glucose and (B) weight loss correlated with longer survival. Linear regression analysis of day 7 blood glucose and percent body weight change for all study animals revealed a significant correlation to survival time (p=0.0065 and p=0.0046, respectively). Results were considered significant when p<0.05.

FIG. 38 is a graph showing blood and glucose levels in animals. KD mice showed significantly lower glucose than controls on day 7 (p<0.05) Animals in the KD-Solace, KD-USF, and KD+HBO2T groups had significantly lower blood glucose levels than controls on day 14 (p<0.05).

FIG. 39 is a graph showing animal weight. The graph bars indicate average percent of initial body weight for animals at weeks 2, 4, and 6. Error bars represent ±SEM.

FIG. 40 is a graph showing animals in the treatment groups showed slower tumor growth than controls. Total body bioluminescence was measured weekly as a measure of tumor size; error bars represent ±SEM.

FIG. 41 is a Kaplan-Meier survival plot graph of study groups showing the KD with HBO2T increases survival time in mice with systemic metastatic cancer. Animals receiving KD+HBO2T showed significantly longer survival compared to control animals (p=0.0084).

FIG. 42 is a graph showing β-hydroxybutyrate levels in animals. KD+HBO2T mice had significantly higher blood ketones than controls on day 7 (p<0.001). KD-Solace, KD-USF, and KD+HBO2T mice exhibited a trend of elevated ketones on day 14, but were not significantly different from controls.

FIG. 43 The KD and HBO2T increases survival time in mice with systemic metastatic cancer. (A) Kaplan-Meier survival plot of study groups. Animals receiving KD and KD+HBO2T showed significantly longer survival compared to control animals (p=0.0194 and p=0.0035, respectively; Kaplan-Meier and LogRank Tests for survival distribution).

FIG. 44 is a series of images showing tumor bioluminescence in mice. Tumor growth was slower in mice fed the KD than in mice fed the SD. Representative animals from each treatment group demonstrating tumor bioluminescence at day 21 after tumor cell inoculation. Treated animals showed less bioluminescence than controls with KD+HBO2T mice exhibiting a profound decrease in tumor bioluminescence compared to all groups. The control has a ROI of 2=6.140×10⁶; SD+HBO2T has a ROI of 2=2.601×10⁶; KD has a ROI of 2=1.540×10⁶; KD-HBO2T has a ROI of 2=1.744×10⁶.

FIG. 45 is a graph showing tumor bioluminescence in mice. Tumor growth was slower in mice fed the KD than in mice fed the SD. Total body bioluminescence was measured weekly as a measure of tumor size; error bars represent 6SEM. KD+HBO2T mice exhibited significantly less tumor bioluminescence than control animals at week 3 (p=0.0062; two-tailed student's t-test) and an overall trend of notably slower tumor growth than controls and other treated animals throughout the study.

FIGS. 46(A)-(B) are graphs showing tumor bioluminescence in mice. Tumor growth was slower in mice fed the KD than in mice fed the SD. Day 21 ex vivo organ bioluminescence of SD and KD+HBO2T animals (N=8) in (A) brain, kidney and spleen; and (B) lungs, adipose tissue, and liver. The results demonstrated a trend of reduced metastatic tumor burden in animals receiving the combined therapy. Spleen bioluminescence was significantly decreased in KD+HBO2T mice (*p=0.0266; two-tailed student's t-test). Results were considered significant when p<0.05.

FIG. 47(A)-(B) are graphs showing blood glucose and b-hydroxybutyrate levels in animals. (A) KD-fed mice showed lower blood glucose than controls on day 7 (***p<0.001). Animals in the KD study group had significantly lower blood glucose levels than controls on day 14 (*p<0.05). (B) KD+HBO2T mice had significantly higher blood ketones than controls on day 7 (***p<0.001). Error bars represent 6SEM. Blood analysis was performed with One Way ANOVA with Kruksal Wallis Test and Dunn's Multiple Comparison Test post hoc; results were considered significant when p<0.05.

FIG. 48 is a graphs showing animal weight. Body weight was measured twice a week. Graph indicates average percent of initial body weight animals at days 7 and 14. KD and KD+HBO2T mice lost approximately 10% of their body weight by day 7 and exhibited a significant difference in percent body weight change compared to control animals (*p<0.05; ***p<0.001). Error bars represent 6SEM.

FIGS. 49(A)-(B) are graphs showing (A) glucose and (B) weight change are correlated to survival. Linear regression analysis revealed a significant correlation between day 7 blood glucose and percent body weight change with survival (p=0.0189 and p=0.0001, respectively). Results were considered significant when p<0.05.

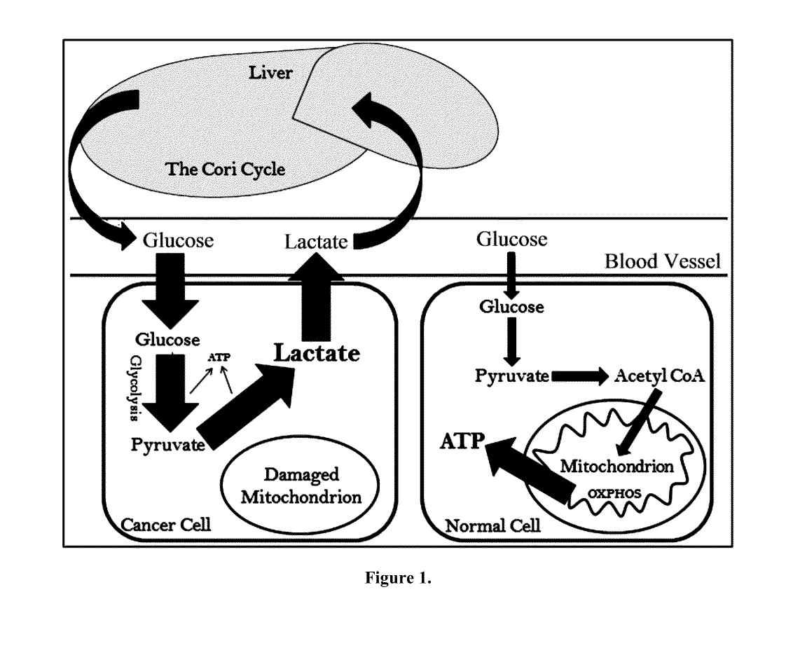

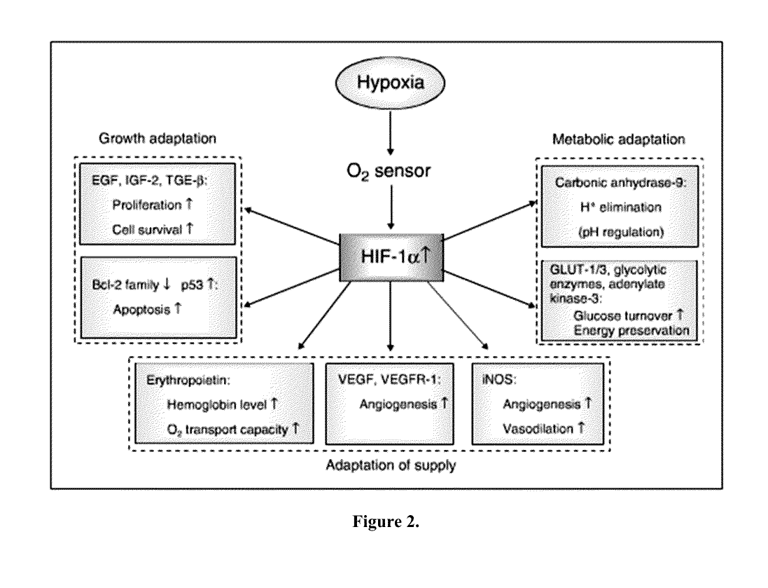

### DETAILED DESCRIPTION OF THE PREFERRED EMBODIMENT

ACA, acetoacetate; AFM, atomic force microscopy; AMPK, adenosine monophosphate kinase ATA, atmospheres absolute pressure (sea level=1 atmosphere, 33 feet of seawater, 760 mmHg); βHB, Beta-hydroxybutyrate; BD, 1,3-butanediol; CO₂, carbon dioxide; CR, calorie restriction; DER, dietary energy restriction; DHE, dihydroethidium; EH-1, Ethidium Homodimer-1; FI, fluorescence intensity; GBM, glioblastoma multiforme; HAFM, hyperbaric atomic force microscopy (AFM inside hyperbaric chamber); H₂O₂, hydrogen peroxide; HBO₂, hyperbaric oxygen; HBO₂T, hyperbaric oxygen therapy; HIF-1, hypoxia inducible factor-1; IGF-1, insulin-like growth factor 1; KD, ketogenic diet; KE, ketone ester; OXPHOS, oxidative phosphorylation; mTOR, mammalian target of rapamycin; MLP< membrane lipid peroxidation; PI3K, phosphoinositide-3 kinase; PO₂, oxygen partial pressure; PTEN, phosphatase and tensin homolog; ROS, reactive oxygen species; .O₂⁻, superoxide anion; Rₐ, average roughness; t, time; SKA, supplemental ketone administration; SLP< substrate level phosphorylation; VEGF, vascular endothelial growth factor.

CNS oxygen toxicity (CNS-OT) is a condition resulting from the harmful effects of breathing molecular oxygen (O₂) at elevated partial pressures, which is known to generate ROS and disrupt brain energy metabolism, which triggers a tonic-clonic seizure. Ketogenesis can be used as a therapeutic strategy to preserve brain metabolism and decrease ROS production in response to toxic levels of hyperbaric oxygen (HBO). Therapeutic ketosis can also be used for a wide range of neuropathologies and cancers resulting from impaired energy metabolism, impaired glucose utilization and elevated levels of oxidative stress.

The etiology of CNS-OT is unknown, but the general consensus is that hyperoxia-induced seizures are triggered by an overproduction of ROS, which disrupts metabolic and ultimately leads to neuronal dysfunction. Therapeutic ketosis counteracts the effects of CNS-OT by a variety of mechanisms, including 1) decreasing ROS production, 2) enhanced mitochondrial efficiency, 3) and by a direct anticonvulsant effect of specific ketones like acetone. Metabolic studies are conducted to determine the precise mechanism of ketone-induced neuroprotection.

Induction of mild ketosis from caloric restriction or the ketogenic diet confers neuroprotection against a wide range of pathologies. Interestingly, the brain's ability to use exogenous ketone bodies for fuel has not been exploited therapeutically. The inventors found that exogenous ketones prevent hyperbaric oxygen-induced seizures in rats, reduce AB-induced oxidative stress in cultured neurons and impair proliferation of brain cancer cells. Results demonstrate that a single intragastric administration of ketone ester in rats (n=12) confers protection from CNS oxygen toxicity (5 ATA O₂) by delaying the latency to seizure from about 16.4±5 minutes (control) to about 79.3 minutes (10 g/kg ketone ester). In the detailed description of preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention. All numerical designations, such as pH, temperature, time, concentration, and molecular weight, including ranges, are approximations which are varied up or down by increments of 1.0 or 0.1, as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

The term “about” or “approximately” as used herein refers to being within an acceptable error range for the particular value as determined by one of ordinary skill in the art, which will depend in part on how the value is measured or determined, i.e. the limitations of the measurement system, i.e. the degree of precision required for a particular purpose, such as a pharmaceutical formulation. For example, “about” can mean within 1 or more than 1 standard deviation, per the practice in the art. Alternatively, “about” can mean a range of up to 20%, preferably up to 10%, more preferably up to 5% and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

Concentrations, amounts, solubilities, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include the individual values and sub-ranges within the indicated range. Thus, included in this numerical range are individual values such as 2, 3, and 4 and sub-ranges such as from 1-3, from 2-4 and from 3-5, etc. This same principle applies to ranges reciting only one numerical value. Furthermore, such an interpretation should apply regardless of the range or the characteristics being described.

“Patient” or “subject” is used to describe an animal, preferably a human, to whom treatment is administered, including prophylactic treatment with the compositions of the present invention. “Patient” and “subject” are used interchangeably herein.

“Ketosis” as used herein refers to an increase in ketone bodies in a subject. Ketosis may improve mitochondrial function, decrease reactive oxygen species (ROS) production, reduce inflammation and increase the activity of neurotrophic factors. Ketosis is safe at levels below about 8 mM and these levels are referred to herein as a nonpathological “mild ketosis” or “therapeutic ketosis”. Ketosis may be due to a ketogenic diet (KD), starvation, or the administration of supplemental ketones.

The term “neurological disorders” as used herein refers to disorders of the central nervous system that are caused by disruptions of brain metabolism. These neurological disorders include, but are not limited to, seizure disorders, Alzheimer's disease, malignant brain cancer including glioblastomas, and traumatic brain injury.

The term “cancer”, “tumor”, “cancerous”, and malignant” as used herein, refer to the physiological condition in mammals that is typically characterized by unregulated cell growth. Examples of cancer include, but are not limited to, brain cancer including tumors in neural tissue such as gliomas, glioblastomas, neuroblastomas, neuroepitheliomatous tumors, and nerve sheath tumors.

“Administration” or “administering” is used to describe the process in which individual ketone esters or any combination of ketone esters thereof of the present invention are delivered to a subject. The composition may be administered in various ways including oral, intragastric, and parenteral (referring to intravenous and intra-arterial and other appropriate parenteral routes), among others. Each of these conditions may be readily treated using other administration routes of ketone esters or any combination thereof to treat a disease or condition.

The “therapeutically effective amount” for purposes herein is thus determined by such considerations as are known in the art. A therapeutically effective amount of individual ketone esters or any combination thereof is that amount necessary to provide a therapeutically effective result in vivo. The amount of ketone esters or any combination of ketone esters thereof must be effective to achieve a response, including but not limited to total prevention of (e.g., protection against) and to improved survival rate or more rapid recovery, or improvement or elimination of symptoms associated with seizure disorders, neurological disorders, cancer or other indicators as are selected as appropriate measures by those skilled in the art. In accordance with the present invention, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of a mammal and the route of administration.

The amount of the ketone ester will depend on absorption, distribution, metabolism, and excretion rates of the ketone ester as well as other factors known to those of skill in the art. Dosage values may also vary with the severity of the condition to be alleviated. The compounds may be administered once, or may be divided and administered over intervals of time. It is to be understood that administration may be adjusted according to individual need and professional judgment of a person administrating or supervising the administration of the compounds used in the present invention.

The dose of the ketone esters administered to a subject may vary with the particular ketone ester, the method of administration, and the particular disorder being treated. The dose should be sufficient to affect a desirable response, such as a therapeutic or prophylactic response against a particular disorder or condition.

Studies of primary cultured cortex neurons fluorescence microscopy with dihydroethidium confirmed that superoxide anion production (measured as DHE fluorescence units) decreased significantly with ketone treatment (2 mM ketones). Superoxide anion production was 27% lower in hyperoxia-treated cultures and 24% lower in Aβ₁-42 treated cultures. Ketone treatment in brain cancer cells (U87MG cultures) significantly reduced cell proliferation (39%) and viability, as assessed by ethidium homodimer-1 staining.

### Clinical Considerations

There has been much confusion about ketosis in the medical community, especially the metabolic function of ketones (VanItallie and Nufert 2003). Many of these concerns result from viewing ketones as “metabolic poison” and the association of therapeutic ketosis with diabetic ketoacidosis (DKA). The pathological state of DKA produces “runaway ketosis” and results in ketone concentrations of 20 mM or greater, but is quickly reversed with insulin administration. A major concern that frequently arises with regards to ketosis is related to the mild metabolic acidosis caused by the accumulation of ketone bodies in the bloodstream. Normal blood pH range is 7.35 to 7.45, and may transiently drop lower during the initial stages of ketosis (Withrow 1980). However, blood pH typically rebounds into normal range as long as ketones are maintained <10 mM (Withrow 1980). The KE data and others (Ciraolo et al. 1995; Desrochers et al. 1995; Puchowicz et al. 2000) have demonstrated that the mild H⁺ load from acute administration of BD-AcAc₂ does not induce a pathological metabolic acidosis. It needs to be determined how the chronic administration of KE influences blood pH. As with the KD, one would expect compensatory metabolic adjustments to buffer the H⁺ load associated with chronic KE-induced ketosis. Furthermore, one would expect chronic KE administration to upregulate ketone transports and further augment the anticonvulsant effects of KE.

### Ketone Esters Treatment

R,S-1,3-butanediol and t-butylacetoacetate were purchased from Sigma (Milwaukee, Wis., USA). All commercial solvents and reagents used were high-purity reagent-grade materials. The KEs synthesized, R,S-1,3-butanediol acetoacetate (BD-AcAc) and R,S-1,3-butanediol acetoacetate diester (BD-AcAc₂), are a non-ionized sodium-free and pH-neutral precursors of AcAc. KEs were synthesized by transesterification of t-butylacetoacetate with R,S-1,3-butanediol (Savind Inc., Seymour, Ill.). The resultant product consisted of a mixture of monoesters and diester, the ratio of which could be adjusted by varying the stoichiometry of reactants. Following synthesis the crude product was distilled under reduced pressure to remove all solvents and starting materials, and the resultant BD-AcAc or BD-AcAc₂ was obtained and assessed for purity using gas chromatography—mass spectrometry (GC-MS).

Hyperbaric Chamber HBO₂ system consisted of two main elements: 1) a plexiglass chamber (˜3 liter capacity, Diamond Box, Buxco, Electronics Inc., model PLY3114), that housed the rat during the experiment, and 2) a hyperbaric chamber (Reimers System Inc.—7.8 ATA MWP), that contained the plexiglass chamber and functioned as the pressure vessel. Both chambers were connected to an air compressor (oil-less rotary scroll compressor—model DK6086, Powerex).

During each experiment (hyperbaric hyperoxia), both the main chamber and the animal chamber were filled with air (dive profile). Rats were placed into the plexiglass chamber and allowed ten minutes to acclimate, at which time the plexiglass chamber was flushed with 100% 02. The animal was then allowed 15 minutes to acclimate before both chambers were compressed to 5 ATA (58.8 PSIG) at a rate of 0.7 ATA/min. The outer chamber was pressurized using air (capacity ˜205 liters) to minimize the risk of an electrical-induced fire. Each experiment was visually monitored via a live camera. LS was calculated from the moment at which the internal and the external chambers reached 5 ATA until the onset of convulsions, identified as high-amplitude, high frequency spikes lasting 10 to 30 sec, followed by polyspikes and wave formation concurrent with tonic-clonic motions of forelimbs and head. After the onset of seizures, the plexiglass chamber was flushed with air to quickly terminate seizure, and both chambers decompressed to sea level. Decompression rate was 1 ATA/min. Rats were then allowed a 15 min recovery period in air at 1 ATA before being removed from the chamber.

The radiotelemetry system consisted of an implantable 4ET radio-transmitter able to amplify and broadcast signals via a receiver (DSI PhysioTel, model RPC-2) connected to an acquisition interface unit (ACQ 7700 Ponemah) via electrical penetrations in the wall of the hyperbaric chamber. The acquisition interface unit was connected to a computer for real time data collection and storage. The same acquisition unit also recorded chamber pressure and temperature, which were measured, respectively, by a thermocouple and pressure gauge directly connected to the acquisition system via BNC (Bayonet Neill-Concelman) cables.

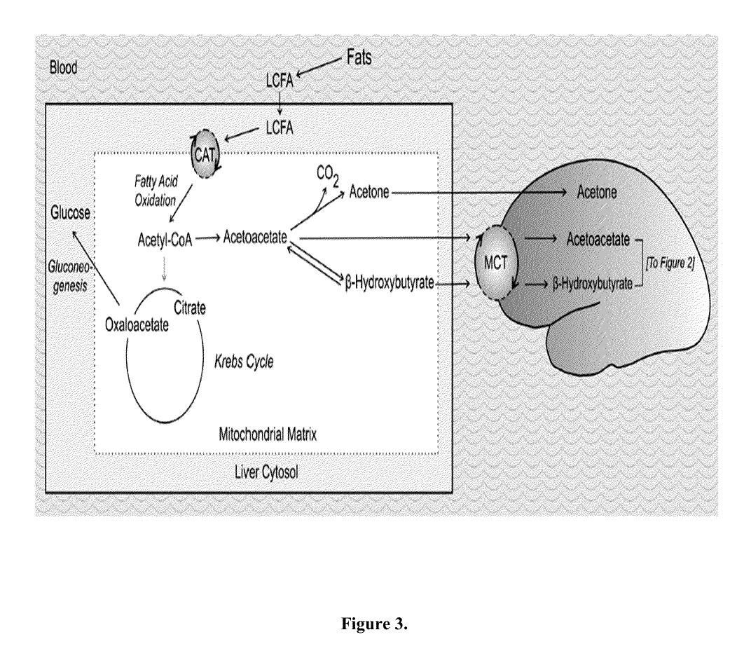

Ketogenic diets (KDs), calorie restriction (CR) and ketogenic precursors (e.g. ketone esters) increase ketone body formation. Ketone bodies represent alternative energy substrates for brain metabolism with anticonvulsant and neuroprotective properties. Acetone readily crosses the blood brain barrier (BBB), whereas acetoacetate and β-hydroxybutyrate were transported via the monocarboxylic acid transporter (MCT) as illustrated in FIG. 3.

### Example 1

Effect of ketones on superoxide production in neurons treated with Aβ₁₋₄₂ and HBO and cell viability of U87MG cells was examined Primary dissociated neuronal cultures of the hippocampus and cortex were acquired from Brain Bits LLC, to increase time efficiency and to minimize cost associated with purchasing rats. Hippocampal or cortical tissue from Brain Bits (shipped in Hibernate®) were enzymatically and mechanically dissociated via pipette trituration. Neurons were plated on 12 mm glass coverslips and allowed to adhere for 1-2 hrs in an incubator maintained at 9-20% O₂ in a humidified atmosphere. Cultures were maintained in media purchased from Brain Bits, including NbActiv1® and NbActive4. After incubation for 7 to 21 days the neurons were placed in the cell chamber on the stage of the hyperbaric imaging system and gently superfused (0.5 ml/min) with aCSF equilibrated with the test level of 02. For experimental protocols cell cultures were maintained in artificial cerebrospinal fluid (aCSF in mM: 125 NaCl, 3.5 KCl, 1 CaCl₂, 1 MgCl₂, 24 NaHCO₃, 0.6 NaH₂PO₄, and 15 glucose) equilibrated with a range of O₂ levels (from 0.09 to 5.0 ATAO₂).

Presence of intracellular ROS is measured by detection of superoxide anion using Dihydroethidium (DHE). Cells were exposed to HBO₂ (5 ATA O₂). Following treatment, cells were incubated in 5 μM DHE for 10 minutes in the dark. DHE is permeable to the cell membrane and freely enters the cell where it reacts with superoxide anion to produce the oxidized ethidium. Ethidium intercalates into the DNA and fluoresces red with an excitation/emission of 485/515 nm. Cells were washed in PBS and then visualized using fluorescent microscopy or quantified using spectrophotometry.

Cells were stained with fluorescent probes for use with fluorescence and confocal microscopy (Invitrogen) as follows:

Dihydroethidium, DHE (1-10 μM; Exλ 525, Emλ 590) detects intracellular .O₂⁻ generation (Bindokas et al. 1996; D'Agostino et al. 2007).

Calcein-AM (4 μM Exλ, 490, Emλ, 535) detects cell volume and monitors cell viability (Crowe, 1995; Inglefield, 1998; Inglefield, 1999).

Ethidium Homodimer-1, EH-1 (6 μM; Exλ, 525, Emλ, 590) enters cells upon membrane damage and thus labels dead or dying cells (Bickler and Hansen 1998; Pinheiro et al. 2006).

Acquisition and statistical analyses of fluorescence imaging was performed as previously reported (D'Agostino, 2007; Filosa, 2002; Ritucci, 1996; Ritucci, 1997; Ritucci, 1998; Crowe, 1995; Weinlich, 1998; Inglefield, 1998; Inglefield, 1999). Average fluorescence intensity (FI) for each cell is calculated as the percent change in fluorescence from baseline, ΔFI=(1−FI/FI_{b})×100, where FI_{b} is the basal fluorescence defined by the two images preceding the experimental recordings. Each cell serves as its own control. Statistical differences between control data and hyperoxic data were tested using ANOVA and the appropriate multiple comparisons post hoc test (P<0.05). All FI values were reported as the mean±SEM. Differences between measured values or between groups were determined using the Student's t-test analysis at the P<0.05 significance level.

Presence of intracellular ROS is measured by detection of superoxide anion using Dihydroethidium (DHE). Cells were exposed to HBO₂ (5 ATA O₂). Following treatment, cells were incubated in 5 μM DHE for 10 minutes in the dark. DHE is permeable to the cell membrane and freely enters the cell where it reacts with superoxide anion to produce the oxidized ethidium. Ethidium intercalates into the DNA and fluoresces red with an excitation/emission of 485/515 nm. Cells were washed in PBS and then visualized using fluorescent microscopy or quantified using spectrophotometry.

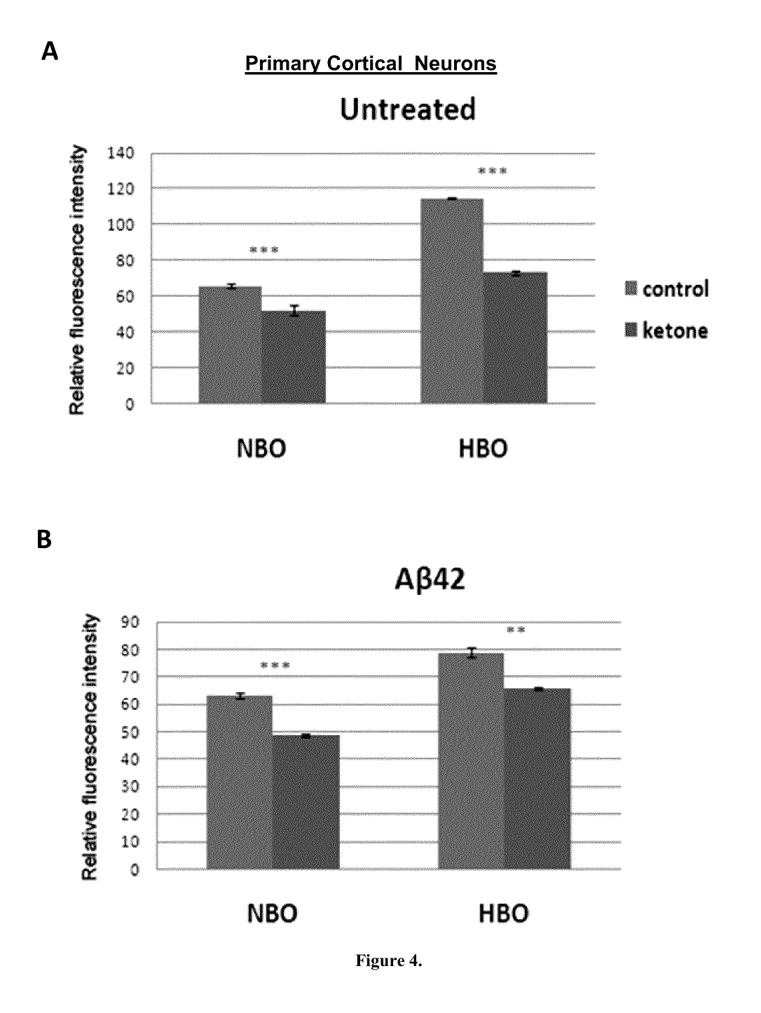

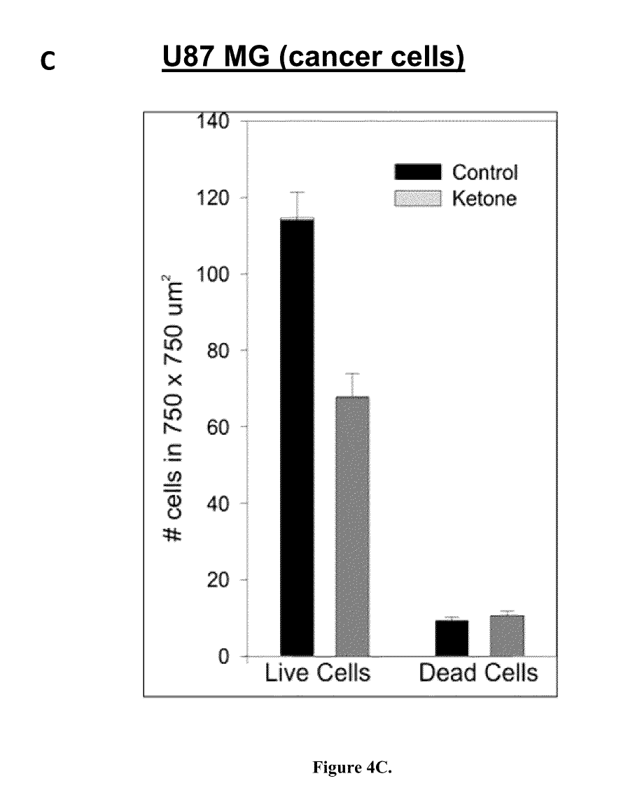

In studies of primary cultured cortex neurons fluorescence microscopy with dihydroethidium confirmed that superoxide anion production (measured as DHE fluorescence units) decreased significantly with ketone treatment (2 mM ketones). FIG. 4(A) shows superoxide anion production was significantly lower in ketone treated cells under normobaric pressure (NBO) and hyperbaric pressure (HBO). FIG. 4(B) shows that in the case of Aβ₁₋₄₂ treated cells a significant reduction of ROS production was observed in NBO and HBO groups treated with ketones. FIG. 4(C) shows the total number of dead (ethidium homodimer-1) U87 cells was similar between groups, but the percentage of live (calcein) cancer cells significantly decreased in ketone-treated (2 mM ketones) cultures. (n=30 culture dishes/group; *, P<0.05). These results implicate the applicability of supplemental ketones as a therapy for neurological disorders in which AB is implicated such as Alzheimer's disease. Ketones protect neurons from oxidative stress, but increase cell death in cancer cells, which cannot use ketones as a metabolic fuel due to defective mitochondria.

Superoxide anion production was 27% lower in hyperoxia-treated cultures and 24% lower in Aβ₁₋₄₂ treated cultures. Ketone treatment in brain cancer cells (U87MG cultures) significantly reduced cell proliferation (39%) and viability, assessed with ethidium homodimer-1 staining These results implicate the applicability of supplemental ketones as a potential therapy for brain cancer.

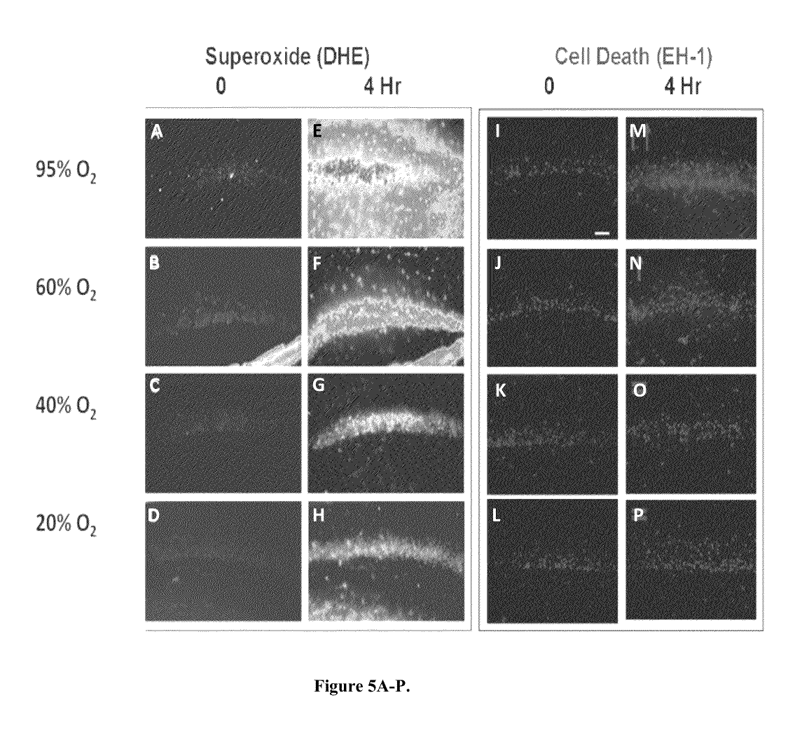

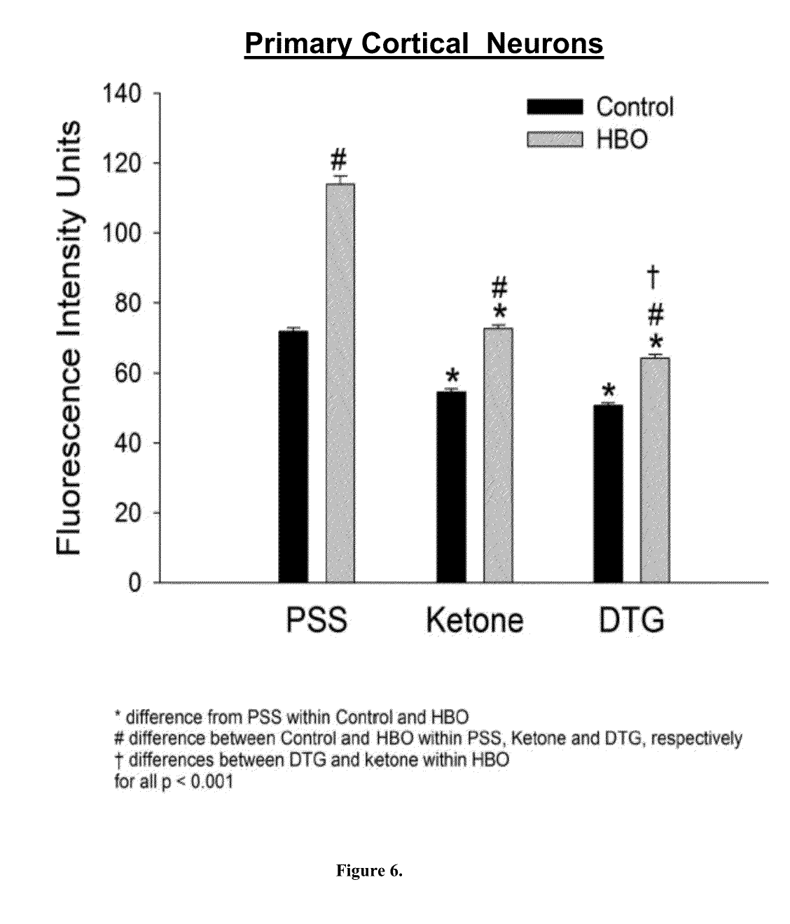

Brain images illustrating superoxide production in CA1 division of hippocampus exposed to graded levels of oxygen were shown in FIGS. 5(A)-(P). Ketones were found to protect cells from hyperoxia-induced oxidative stress as shown in FIG. 6. Primary cortex neurons grown for 10 days under normal conditions were exposed to acute hyperoxia (60 min, 5 ATA O₂). This caused a significant increase in superoxide anion production. Ketone treatment decreased baseline superoxide production in a way that resembled the effect of the neuroprotective drug DTG. Both ketones and DTG prevented the hyperoxia-induced increase in superoxide production (n=110 cells analyzed/condition, * indicates p≦0.005).

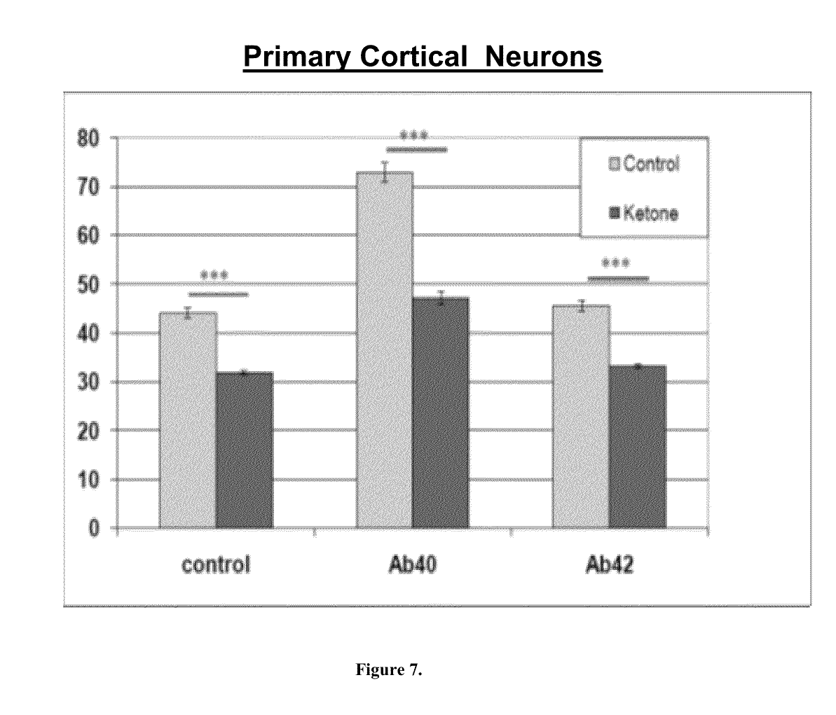

The effect of ketones on superoxide anion production in primary cortex neurons is shown in FIG. 7. Ketones reduced oxidative stress in primary cultured neurons exposed to the proteins implicated in Alzheimer's disease. These results indicate that the administration of supplemental ketone esters can be used as a potential therapy against Alzheimer's disease.

### Example 2

Anticonvulsant effect of supplemental ketones was tested in rats exposed to hyperbaric oxygen (5 ATA O₂). The effects of ketone esters (KEs) in preventing CNS-OT in rats were assessed before, during and after HBO₂ exposure by measuring various parameters.

Adult male Sprague-Dawley rats (n=60) rats (300-450 grams; 3 to 6 month old) were obtained from Harlan, anesthetized in 3-5% isoflurane (in 02) and implanted with a 4ET radio-transmitter (Data Sciences International, DSI) using sterile surgical technique. The rat chamber was ventilated with pure O₂ while the hyperbaric chamber, containing the radio-receiver (DSI), was pressurized in parallel with air to 5 atmospheres absolute (ATA). One pair of leads (positive and negative poles) was implanted in the costal diaphragm at the junction with the abdominal wall for diaphragmatic electromyogram (dEMG) signals, one pair of electrodes was inserted in the pectoral muscle to acquire electrocardiogram (ECG) data, and two pairs of wires were embedded in the skull between Bregma and Lambda, with one lead on either side of midline for each pair (EEG recordings). The EMG wires were not inserted into crural diaphragmatic muscle because of the high risk of pneumothorax due to the thinness of the muscle (419 to 630 μm). 4ET radio-transmitters also monitored core body temperature and physical activity. Rats were weighed immediately before surgery and subsequently once every 7 days, just prior to the weekly exposures to HBO₂. After surgery, every animal recovered for ≧1 week. The rats were food (not water) deprived for 18 hours prior to the start of the experiment. Test substances of distilled water (control), BD (10 g/kg) or BD-AcAc₂ (10 g/kg) were administered by 3 ml oral gavage (this was time 0).

Each rat underwent two dives at 5 ATA O₂ in the hyperbaric chamber, consisting of control (water gavage) and treatment, including R,S-1,3-butanediol AcAc diester (BD-AcAc₂) and R,S-1,3-butanediol (BD) given in random order. Data showed that BD-AcAc₂ was the most effective KE against CNS-OT. In each case animals were gavaged about 30 minutes prior to diving. Total ketones were significantly elevated (>5 mM) about 30 minutes after gavaging BD-AcAc₂. One week after the control dive, the same rats were dived following treatment. Subsequent exposure to HBO₂, blood ketones and blood glucose were assayed using a blood glucose/ketone monitor (NovaMax Plus), commercially available kits (Caymen Chemical) or assayed at the metabolomics core facility at Case Western Reserve.

Table 1 depicts the CNS-OT Prevention Protocols

| Acute Treatment | Control | Dose/volume/freq. |
|---|---|---|
| (ketogenic precursor) | (water) | (gavage) |
| R,S-1,3-butanediol AcAc diester | 1-3 ml | 5-10 g/kg/3 ml/one dose |
| (BD-AcAc₂) | | |
| R,S-1,3-butanediol | 1-3 ml | 5-10/kg/3 ml/one dose |
| (BD) | | |

Whole blood samples (10 μl) were acquired for analysis of glucose and BHB utilizing a commercially available glucose/ketone monitoring system (Nova Max® Plus) at time 0, 30, 60, 120, 180 and 240 min. In addition, heparinized blood samples (200 μl) were collected into Eppendorf tubes at time 0, 30, 60, 120, 180 and 240 min. Samples were processed for the detection and quantification of BHB, AcAc, and acetone. Briefly, samples were chilled on ice for 30 s, centrifuged in a micro-centrifuge (13,000 G) for 3-5 min and plasma (>100 μl), treated with reducing reagent of cold 0.2M sodium borodeuteride (NaBD₄; Sigma, 205591, CAS 15681-89-7) dissolved in 0.1M NaOH (8.4 mg NaBD₄ in 1 ml of 0.1M NaOH) and then immediately frozen on dry ice before storing at −80° C. Acetone was analyzed at the 60 minute time point, which was the predicted peak of blood AcAc levels (Desrochers et al. 1995). 300 μl of whole blood were collected in addition to the above collections, stabilized with cold 0.2M NaBD₄, and then immediately frozen on dry ice. Samples were stored at −80° C. until analyzed for ketones. Internal standards of [²H₆]BHB or [²H₈]isopropanol were added to the treated plasma or blood samples (50 μl or 15 μl) and the BHB, AcAc (as M+1 of BHB) or acetone (as 2-propanol) metabolites were analyzed by gas chromatography-mass spectrometry (GC-MS) using an Agilent 5973 mass spectrometer, linked to a 6890 gas chromatograph equipped with an autosampler. Briefly, GC-MS conditions were either EI or CI mode (electron or chemical ionization mode); the samples were detected by selected ion monitoring as the BHB- and AcAc-trimethylsilyl derivatives (EI) or the derivative of acetone-pentafluorobenzoyl (CI).

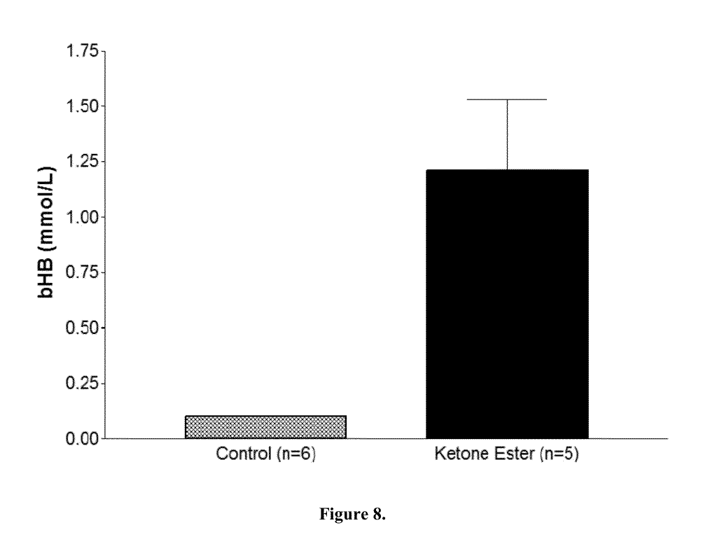

Blood levels of the ketone β-hydroxybutyrate following oral administration of ketone ester were illustrated in FIG. 8. As shown in the figure, within 30 minutes levels of blood ketones rose above 1 mM. The neuroprotective effect of ketones was proportional to the level of ketogenesis. The test measured only BHB, but it is estimated that total ketones (including acetoacetate) were approximately twice as high (˜2.5 mM). Safe levels of ketosis were typically under about 8 mM.

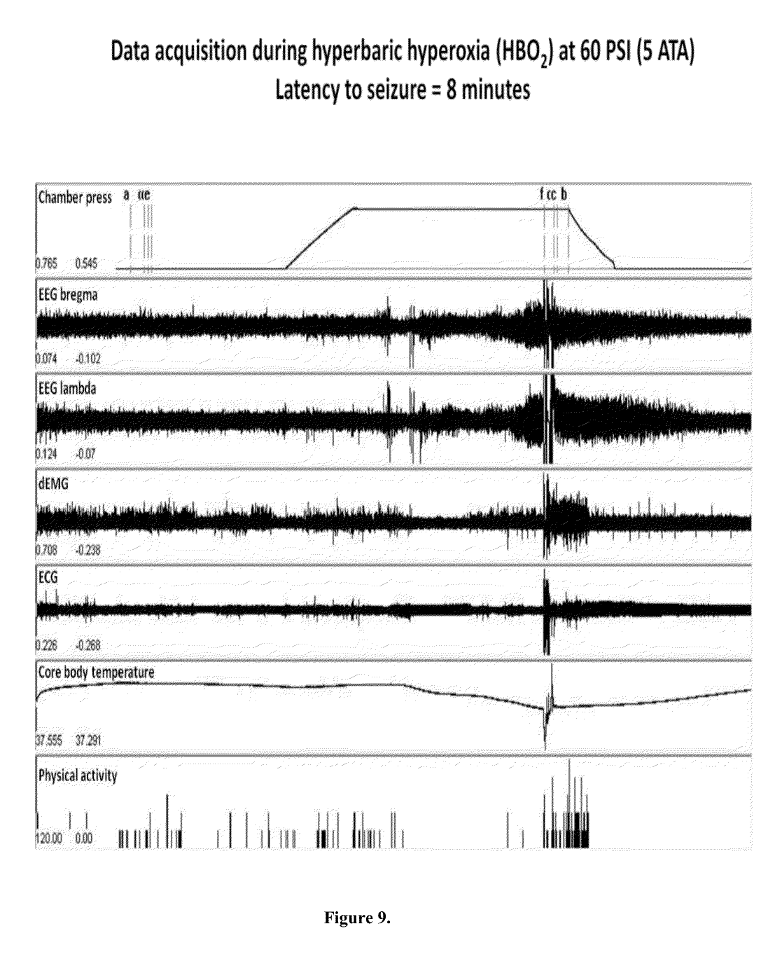

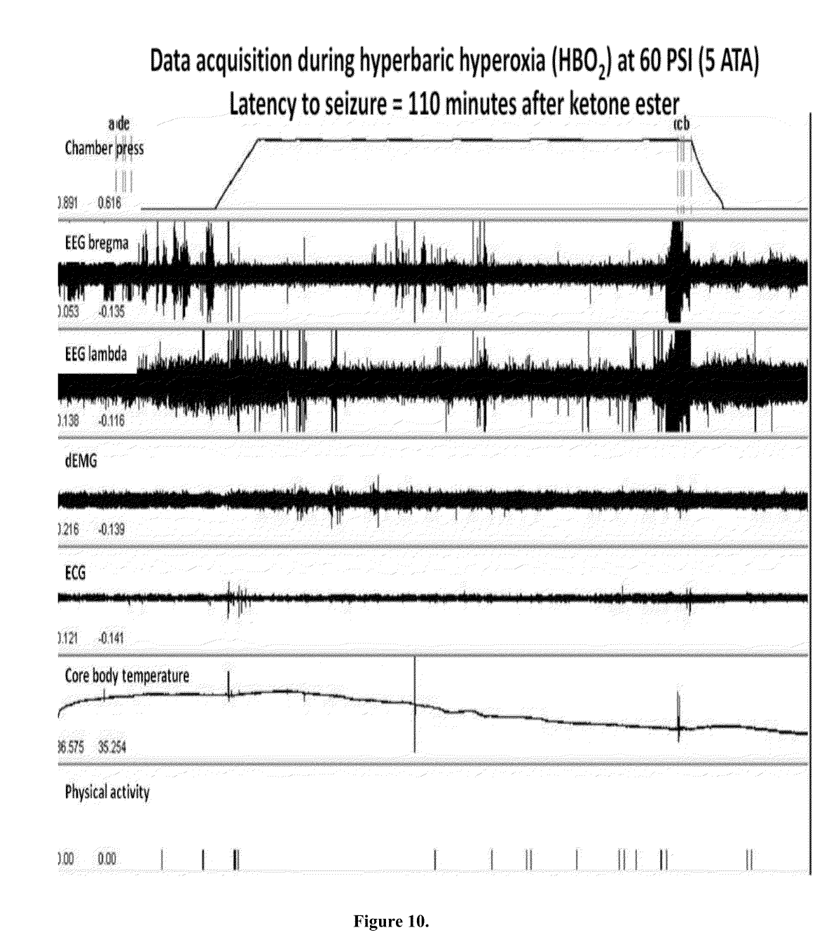

Data acquisition during HBO₂ at 60 PSI (5 ATA) is shown in FIG. 9. FIG. 9 illustrates raw data of a rat exposed to HBO₂ with a latency to seizure time of equal to about 8 minutes. When the same rat was given ketone ester, the animal resisted seizures from HBO₂ for about 110 min, as seen in FIG. 10.

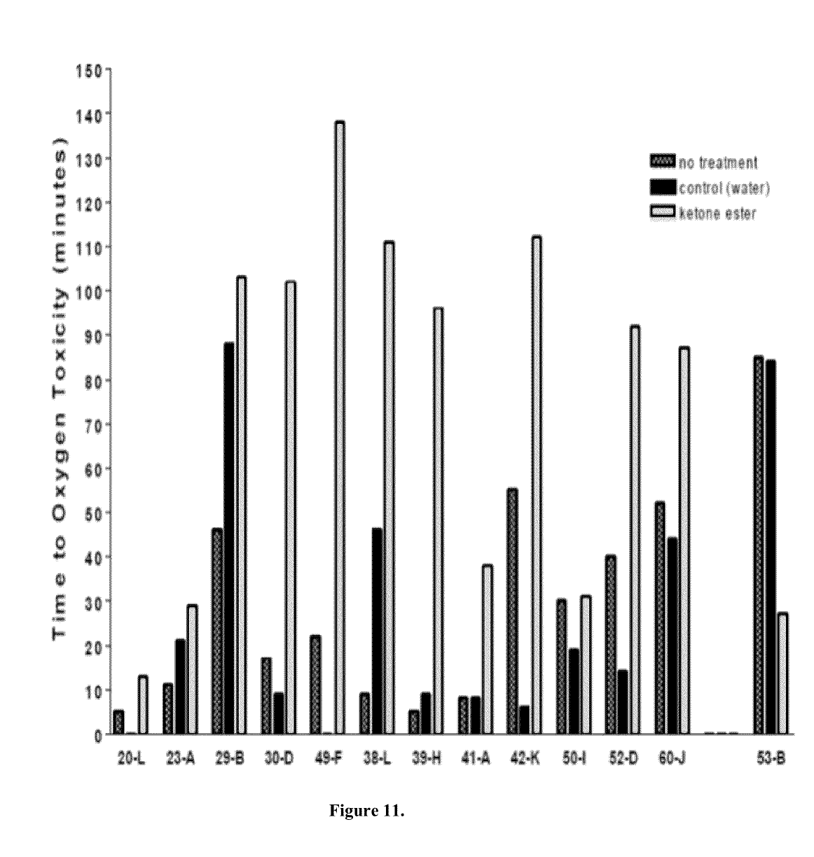

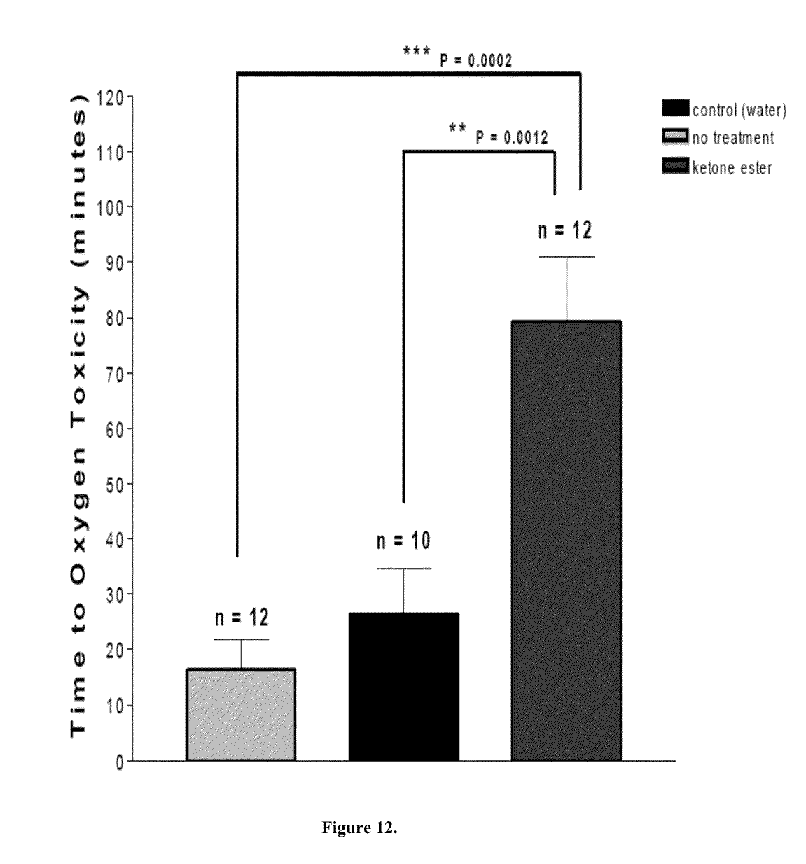

Responses from individual rats with no treatment, control (water) and ketone ester treatment are illustrated in FIG. 11. Administration of ketone ester (˜3 mL) about 30 minutes prior to exposure to HBO₂ (5 ATA O₂) significantly increased the latency time to seizure, as seen in FIG. 12. Average time to seizure due to HBO₂ was measured as the time to the first electrical discharge in the EEG. Intragastric administration of ketone esters, specifically BD-AcAc₂, protected rats against CNS-OT. It was also found that administration of ketone esters (3 mL gavage) about 30 minutes prior to HBO₂ (5 ATA O₂) exposure significantly increased the latency time to first electrical discharge of EEG.

Radio-telemetry physiology experiments confirmed the efficacy of two KEs (R,S)-1,3-butanediol acetoacetate monoester (BD-AcAc) and diester (BD-AcAc₂) in the prevention of CNS-OT in unanesthetized conscious rats. Administration of BD-AcAc and BD-AcAc₂, but not (R)-1,3-butanediol ester (BHB ester) or BD 30 minutes prior to exposure to HBO₂ (5 ATA O₂) significantly increased the latency time to seizure. The standard gavage volume was about 3 ml (˜10 g/kg) for all treatments. All substances were gavaged in about a 3 ml dose (˜10 g/kg). Average time to seizure from exposure to HBO₂ was measured and confirmed with video-EEG in untreated, control (water) and treatment groups. Precursors to AcAc, but not BHB, delayed CNS-OT, occasionally causing onset of pulmonary toxicity (after prolonged HBO₂ exposure).

Direct effect of specific ketones was shown by Chavko et al (1999), which demonstrated that an elevation of the primary ketone body BHB (via 1,3-butanediol injection) did not delay CNS-OT. This observation is consistent with the finding that inducing ketosis by administration of BHB does not prevent seizures in animal models (Bough and Rho 2007). It is well known that BD produces ketosis, but primarily through the generation of BHB, and thus produces only low levels of AcAc and acetone (Tate et al. 1971). However, elevation of AcAc and acetone prevents acutely provoked seizures (e.g. chemical, electrical, audiogenic) in animal models (Likhodii et al. 2008; Rho et al. 2002). Acetone is relatively nontoxic (LD50>5 g/kg; rat) and has an anticonvulsant effect at subnarcotic concentrations (Gasior et al. 2007). Endogenous acetone levels are typically very low unless prolonged starvation is achieved (Cahill 2006). Collectively, these studies demonstrate that AcAc and acetone, but not BHB, have intrinsic anticonvulsant properties in standardized animal models of seizures. The inventors developed and tested a KE that elevated all three ketone bodies, but with the highest potential to elevate and sustain blood levels of AcAc (Ciraolo et al. 1995; Desrochers et al. 1995), which by spontaneous decarboxylation, would elevate acetone.

The data show that preferential utilization of AcAc and acetone, elevated by KE, delays CNS-OT. Evidence exists for a direct effect of these ketone bodies on hyperpolarizing neuronal membrane potential and reducing synaptic release of excitatory neurotransmitters (Yellen 2008). This data support the idea that KATP channels are activated in the presence of ketone bodies (BHB and AcAc), but the mechanism of this activation is largely unknown. Work by Juge et al (2010) demonstrates that AcAc inhibited glutamate release by competing with Cl⁻ at the site of allosteric regulation (Juge et al. 2010). Very little is known about the anticonvulsant mechanism of acetone. Like other solvents, acetone can alter plasma membrane fluidity, which may counteract hyperoxia-induced alterations in plasma membrane function and structure (D'Agostino et al. 2009).

The foregoing results have demonstrated the anticonvulsant effect of boosting ketogenesis and have shown that intragastric administration of ketone esters protects rats against CNS oxygen toxicity (seizures). A dietary supplement of ketone esters can rapidly elevate blood ketones and significantly maintain elevated ketone levels for several hours, even higher than levels achieved with fasting, CR or KD, and without fear of metabolic acidosis associated with diabetic ketoacidosis (DKA). A comparison of ketogenesis from starvation, KD, ketone ester, diabetic ketoacidosis and alcoholic ketoacidosis is shown in Table 2.

Table 2 depicts the comparison of ketogenesis from starvation,
ketogenic diet, ketone ester with the pathological state of diabetic
ketoacidosis (DKA) and alcoholic ketoacidosis (AKA).

| | | Therapeutic Ketosis |
|---|---|---|
| | Diabetic Ketoacidosis | (ketone ester) |
| Blood Ketones (mM) | >10-20 | 0.5-8 |
| Insulin | Dysregulated/Absent | Low |
| Glycemia | High | Low |
| Renal Metabolism | Ketonuria, glycosuria, | Mild osmotic |
| | reduced GFR | diuresis |
| Acidosis | Very high | Mild and regulated |
| Pathology | Hypovolemia, hypotension | None |
| | and death | |
| Cognitive Performance | Impaired | Enhanced |
| Physical Performance | Impaired | Enhanced |

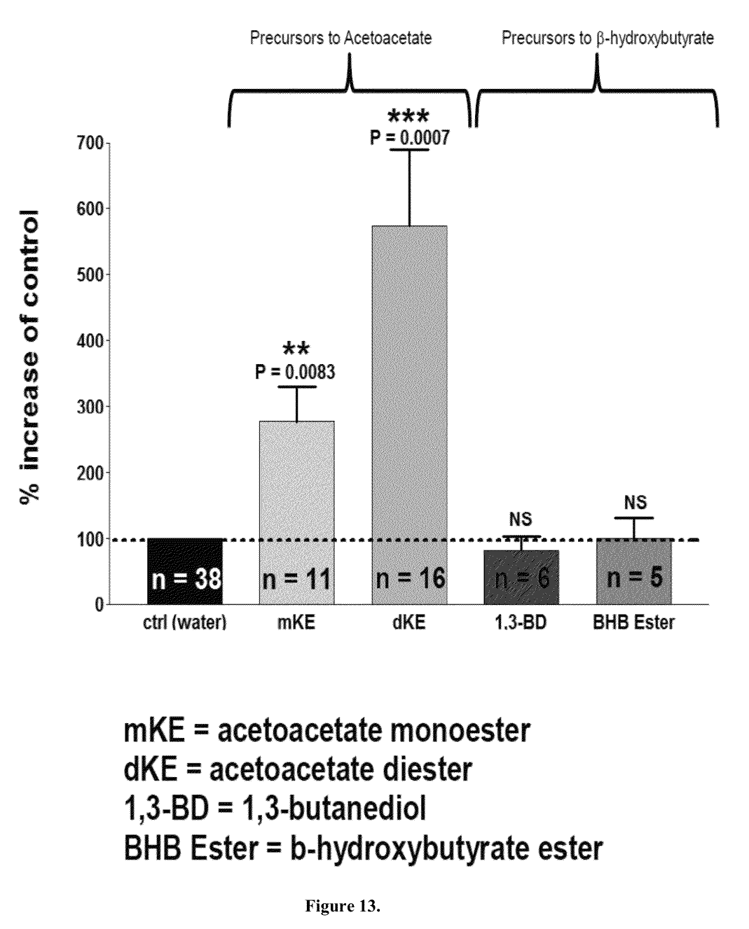

Acute intragastric administration of ketone esters (10 g/kg), a non-ionized precursor to ketone bodies, given 30 min before diving, delayed seizures in rats exposed to 5 ATA O₂, as seen in FIG. 13. Acetoacetate monoester (mKE) and diester (dKE) increased the latency to seizure by 285% and 570%, respectively. 1,3-butanediol and B-hydroxybutyrate ester elevated blood levels of B-hydroxybutyrate, but had no effect on seizure latency. These results demonstrate the anticonvulsant effect of acetoacetate esters. Ketone esters, specifically BD-AcAc₂, increase latency to seizure in rats exposed to 5 ATA O₂. The data indicates increased resistance to oxygen-induced seizures (570 of the esters tested, the AcAc esters which are rich in BD-AcAc₂, provide the most effective neuroprotection against CNS-OT.

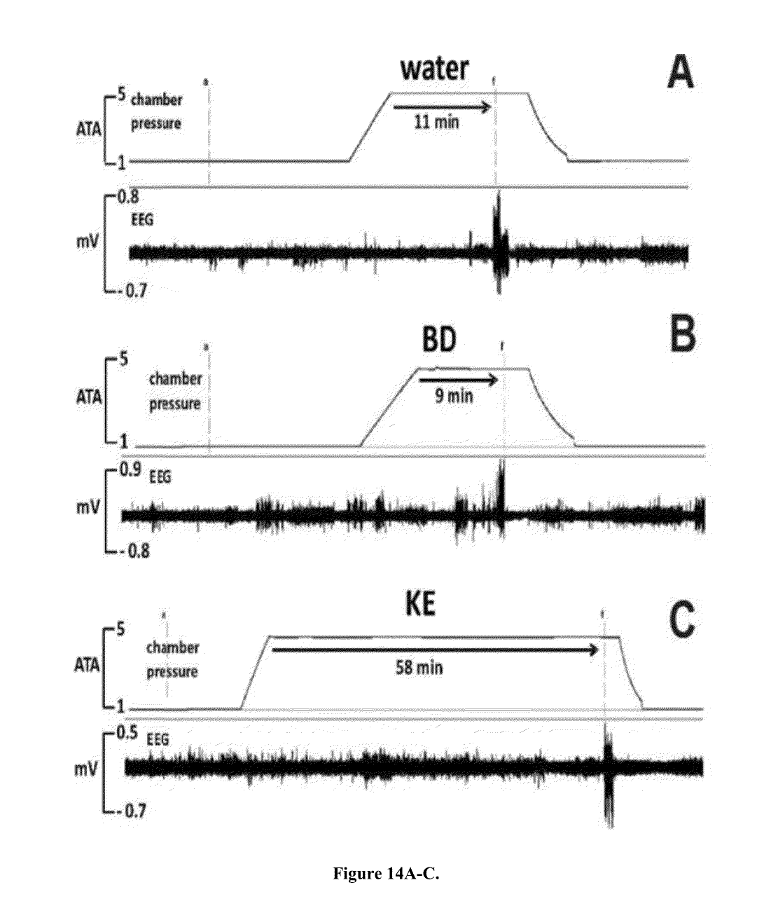

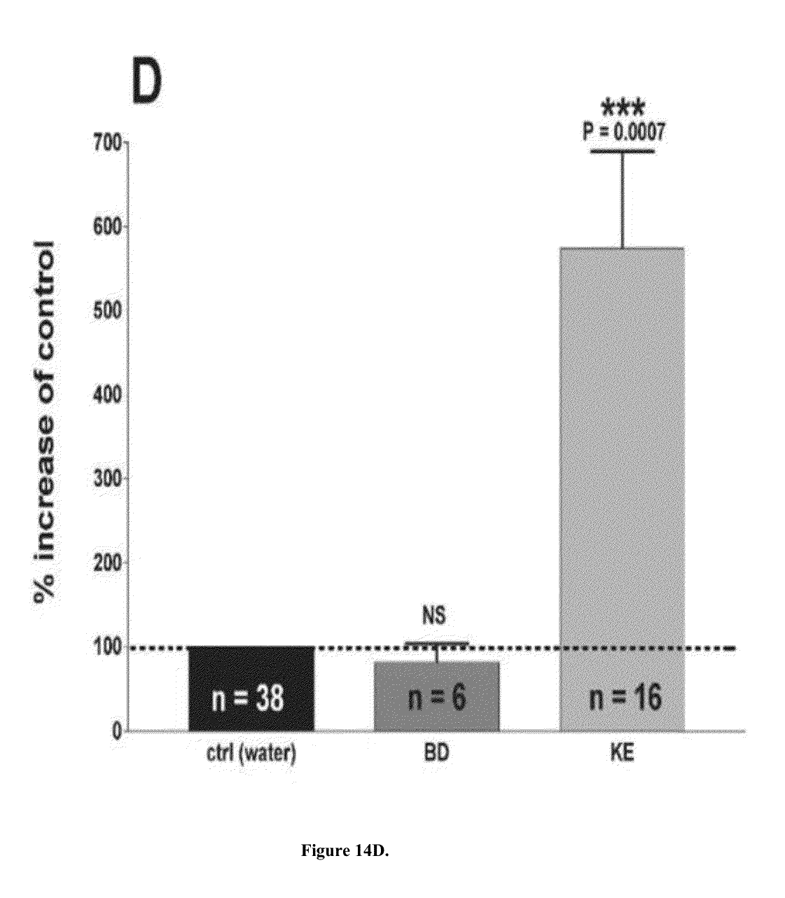

FIGS. 14 (A), (B) and (C) show three examples of real time EEG recordings after intragastric administration of water, BD and KE, respectively. Latency to seizure (LS) was calculated as the percentage increase compared to the control, seen in FIG. 14(D). Following the intragastric administration of KE in 16 rats, the LS was significantly longer (574±115%, P<0.01). In contrast, BD administration did not delay CNS-OT.

As shown above, the inventors tested the potential of KE-induced therapeutic ketosis as a mitigation strategy against CNS-OT seizures. A single oral administration of the KE, BD-AcAc₂, caused: (1) rapid and significant elevations of BHB (>3 mM) and AcAc (>3 mM) that resulted in a sustained elevation of total ketones >6 mM for over 4 hrs; (2) significant elevation in acetone (˜0.7 mM) within about 60 minutes; and (3) increased latency to seizure (LS) >570% compared to control (water) or BD, even though BD caused a significant increase in BHB.

### Example 3

Blood ketones and glucose levels were examined following administration of water,

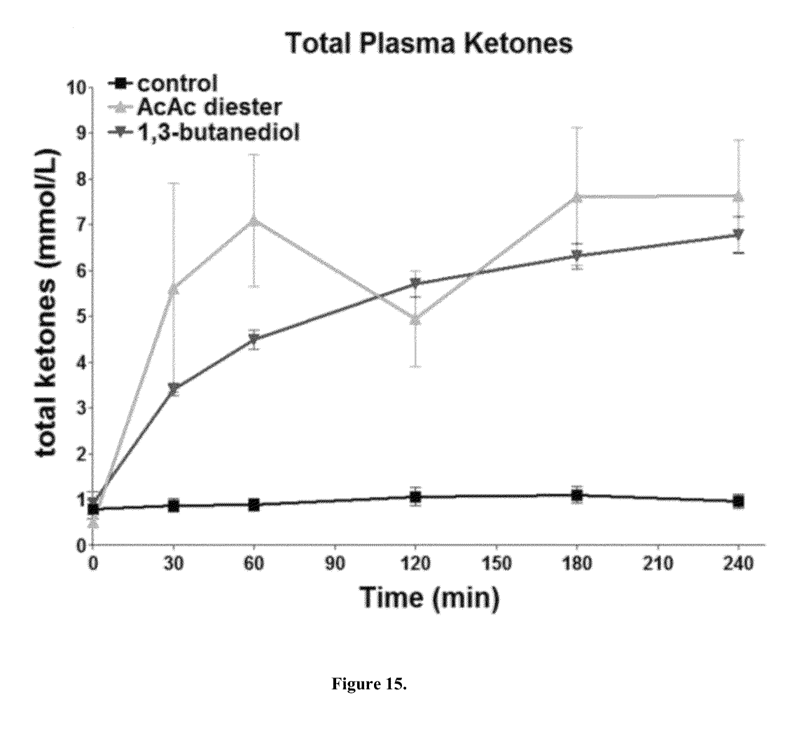

KE and BD. The ketone diester (dKE) was found to cause a rapid and sustained increase in total blood plasma ketones. Blood plasma concentration of total ketones (BHB+AcAc) levels in adult Sprague Dawley rats (n=6 rats/group; 250 to 350 g) semi-fasted (18 hrs) and gavaged with 3 mL (˜10 g/kg) of water (control), BD-AcAc₂) or BD are illustrated in FIG. 15. Blood was collected and processed as described in the previous example.

Ketone measurements were taken at 30, 60, 120, 180 and 240 minutes. Blood plasma was treated with sodium borodeuteride (NaB₂H₄) to stabilize ketone concentration and then assayed by GC-MS. The rapid rise relating to blood ketones from BD-AcAc₂ is due primarily from rapid desterfication in blood and tissues. Desterification of BD-AcAc₂ releases 1,3-butanediol, which is metabolized in the liver to BHB.

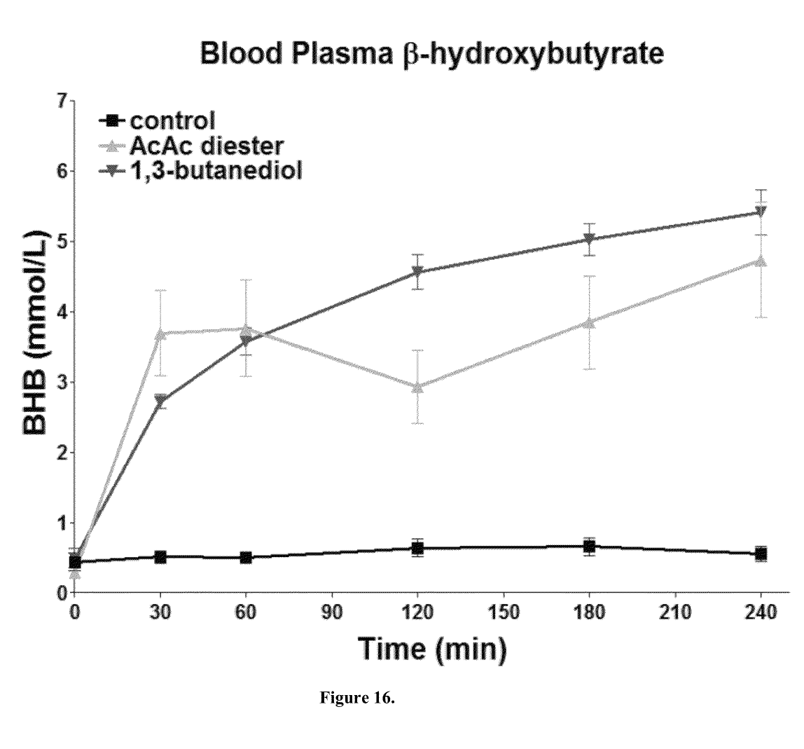

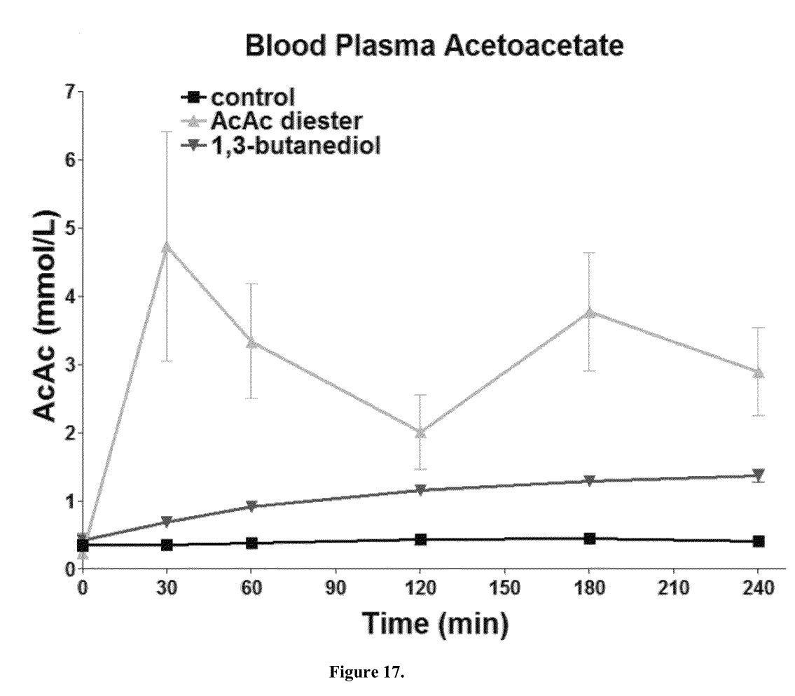

FIG. 16 shows blood plasma levels of BHB in rats (n=6 rats/group) semi-fasted (18 hrs) and gavaged with 3 mL (˜10 g/kg) of water (control), R,S-1,3-Butanediol acetoacetate diester (AcAc Diester) or R,S-1,3-Butanediol. Elevated BHB levels were demonstrated as compared to the control after administration of either ketogenic compound. FIG. 17 shows blood plasma levels of AcAc in rats (n=6 rats/group) semi-fasted (18 hrs) and gavaged with 3 mL (˜10 g/kg) of water (control), BD-AcAc₂ (AKE) or R,S-1,3-butanediol (BD). The results of FIG. 17 illustrate that AcAc level was increased significantly more by KE as compared to water or BD.

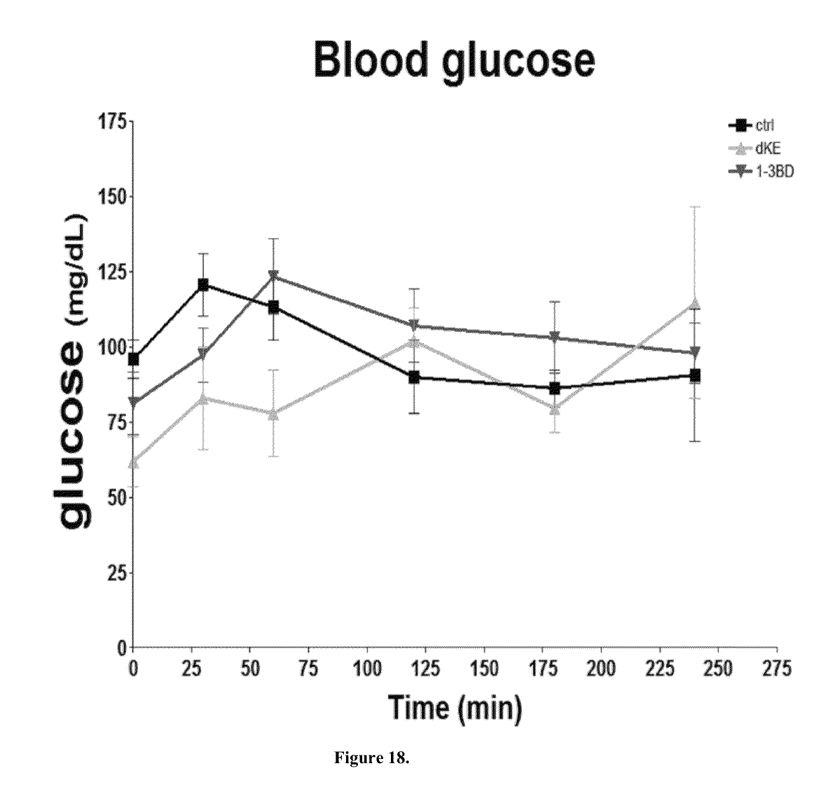

FIG. 18 illustrates that there is no change in blood glucose in all groups in response to BD-AcAc₂, which represents a calorically dense (6 kcal/gram) substance that does not elevate blood glucose. A sharp rise in blood glucose can induce a seizure and stimulate the progression of existing cancer. BD-AcAc₂ represents a novel therapeutic strategy to provide metabolic fuel without increasing blood glucose, which occurs following ingestion of carbohydrates and protein (via gluconeogenesis).

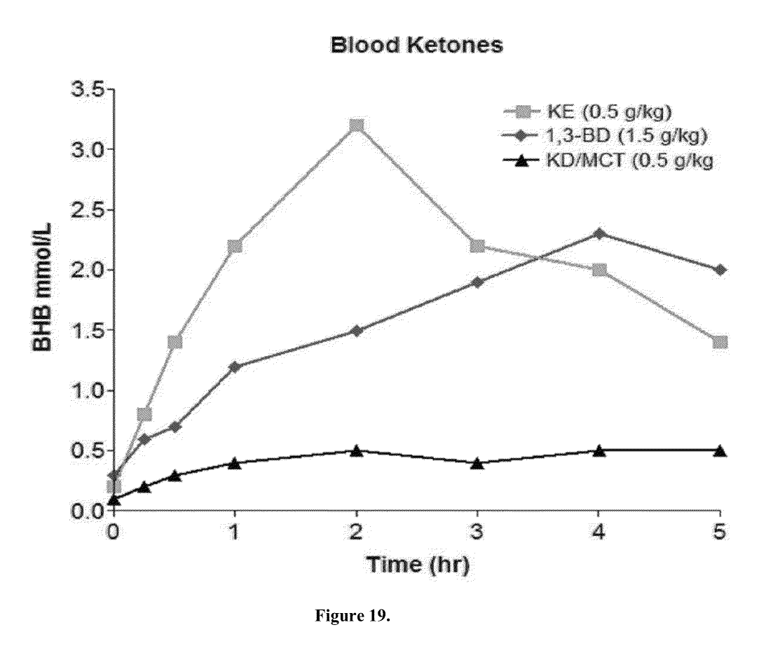

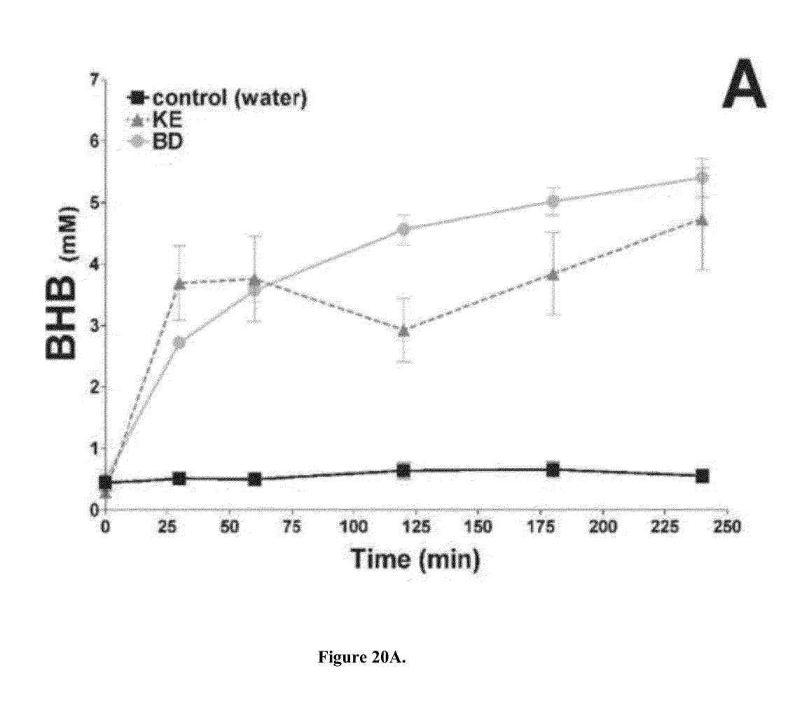

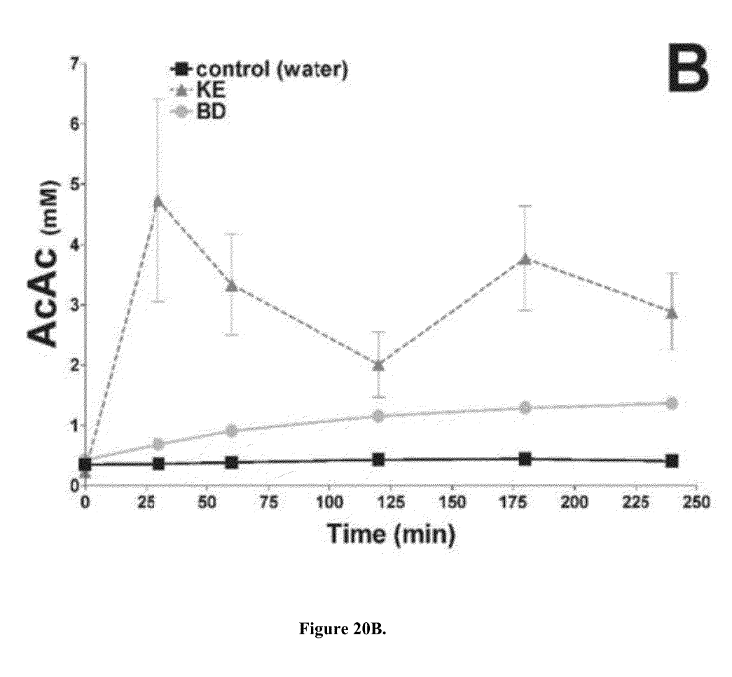

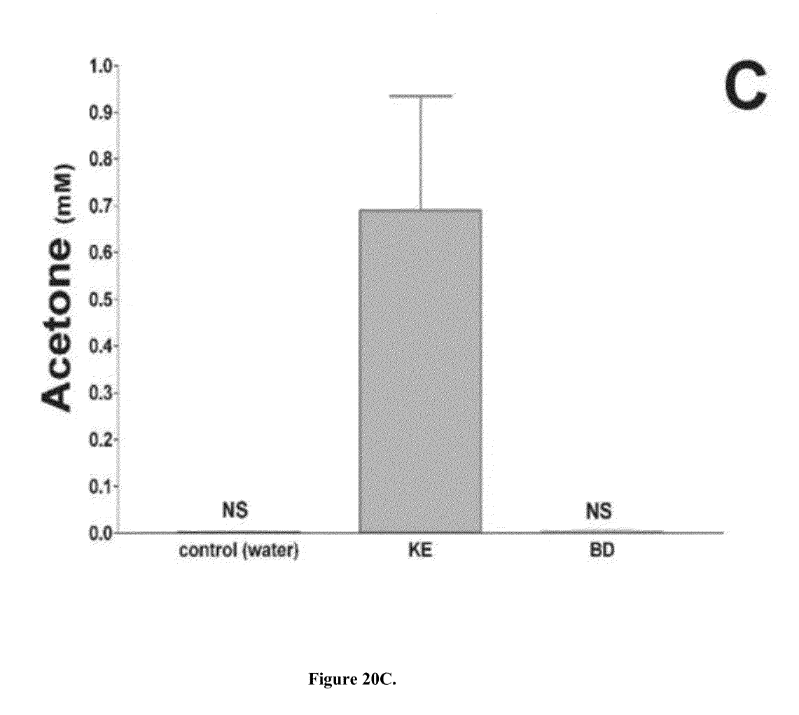

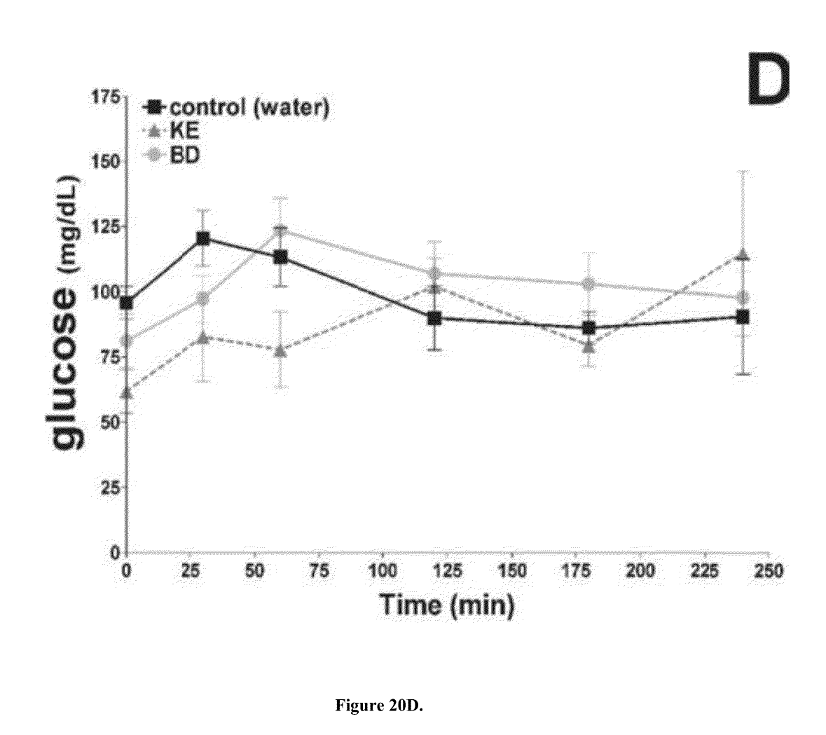

Blood levels of BHB in response to BD-AcAc₂ (KE), 1,3-butanediol (BD) and ketogenic diet (KD) supplemented with MCT oil are shown in FIG. 19. Note the dose of KE relative to 1,3-BD, which required considerably more 1,3-BD to raise BHB levels. KE caused a significant increase in BHB and AcAc at 30 minutes, which remained elevated for 4 hours after intragastric administration, as seen in FIGS. 20(A)-(C). BD administration caused similar elevation in BHB, but only modest elevation in AcAc relative to KE, seen in FIG. 20(B). The breakdown product of AcAc, acetone, was significantly higher at 60 minutes following KE, but not BD administration, seen in FIG. 20(C). In contrast, supplying calories (˜6 kcal/gram) in the form of KE or BD had no significant effect on blood glucose levels relative to control (water) over 4 hrs, as seen in FIG. 20(D).

### Example 4

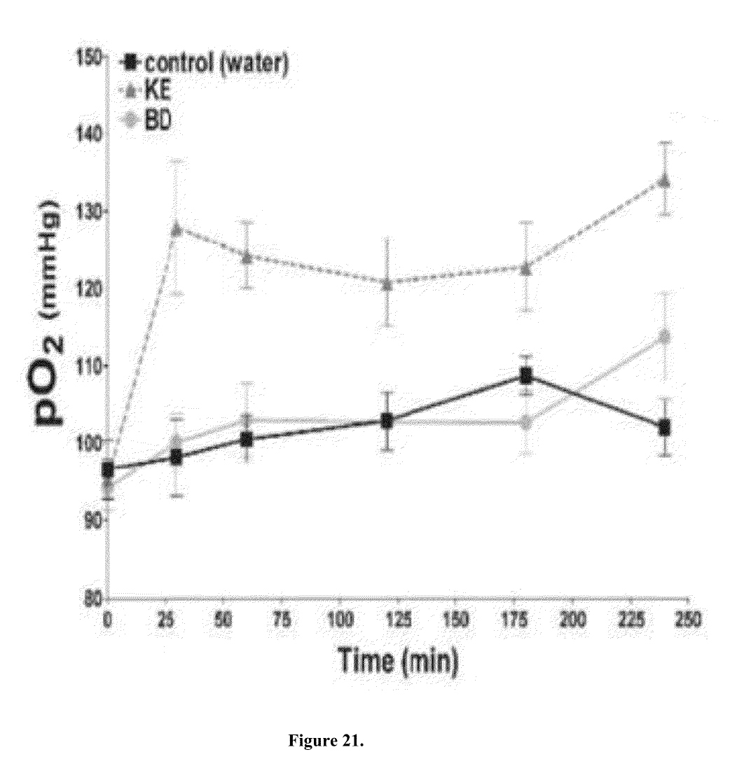

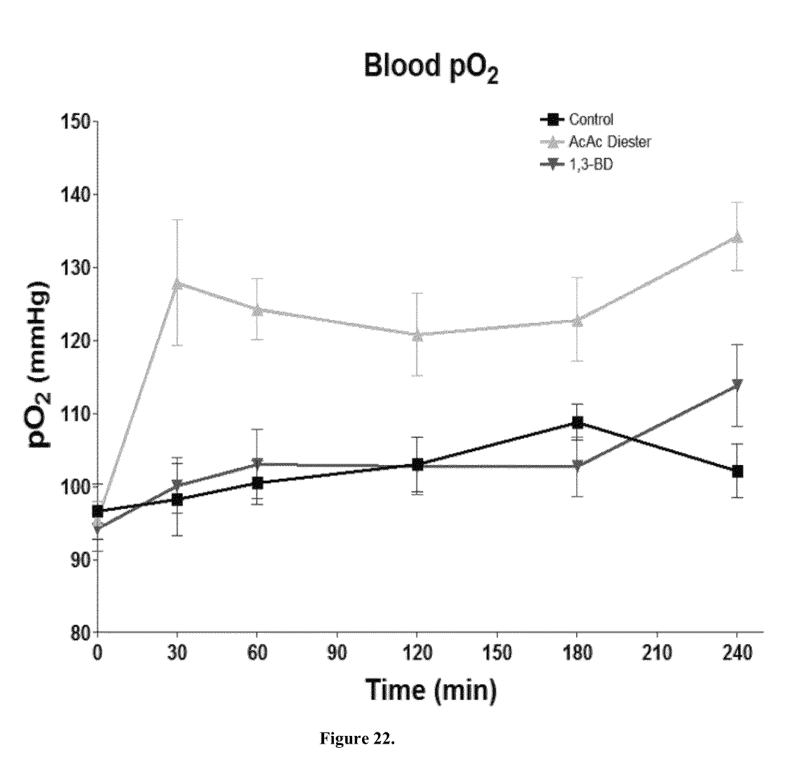

Sprague Dawley rats (n=6 rats/group; 250 to 350 g) were semi-fasted (18 hrs) and gavaged with 3 mL (˜10 g/kg) of water (control), BD-AcAc₂) or BD. 60 μl blood sample was withdrawn at each time point and immediately analyzed with a blood gas analyzer (OPTI CCA-TS© Blood Gas Analyzer, cat #: GD7013—Global Medical Instrumentation, Inc.) for blood pH, pO₂, and pCO₂. Ketone measurements were taken at 30, 60, 120, 180 and 240 minutes. Blood plasma was treated with sodium borodeuteride (NaB₂H₄) to stabilize ketone concentration and then assayed by GC-MS. There were no differences in pO₂ after administration of water or BD, but pO₂ values were considerably higher in KE group and remained relatively hyperoxic (pO₂>120 mmHg) during the 4 hour experiment, as seen in FIGS. 18 and 21. FIG. 22 shows BD-AcAc₂ (KE) improves oxygenation in the blood. BD-AcAc₂ appears to stimulate breathing by augmenting the neural control of autonomic regulation by stimulating acid-sensing neurons. Alternatively, the BD-AcAc₂ possibly reduces oxygen demands and maintain redox balance during hyperoxygenation by enhancing cellular respiration.

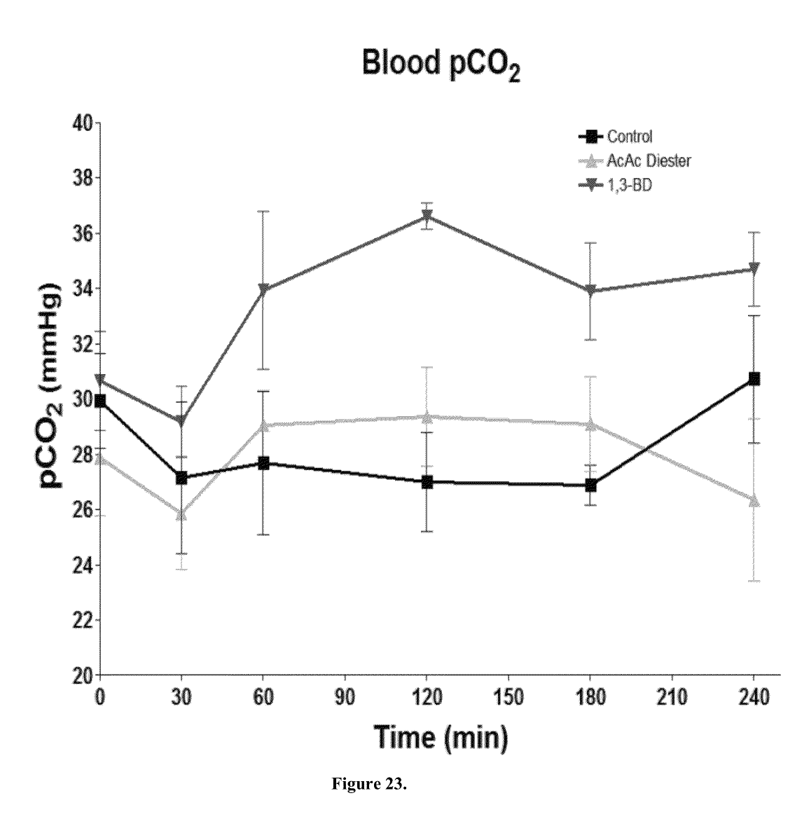

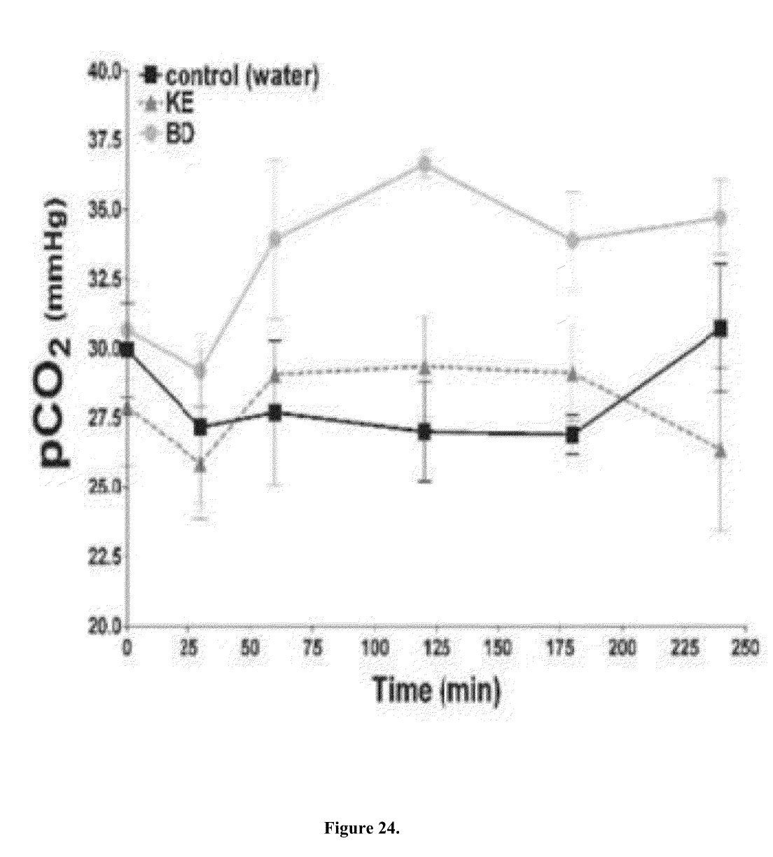

The pCO₂ of control and KE groups were normal, but was significantly higher with BD, although still normocapnic, as seen in FIGS. 23 and 24. FIG. 23 shows that a potential problem with raising blood ketones with 1,3-butanediol is suppression of CNS function due to intoxication from the di-alcohol. The increased CO₂ with BD may be due to a depression in the neural control of respiration. BD-AcAc₂ raises blood ketones without causing an increase in blood pCO₂.

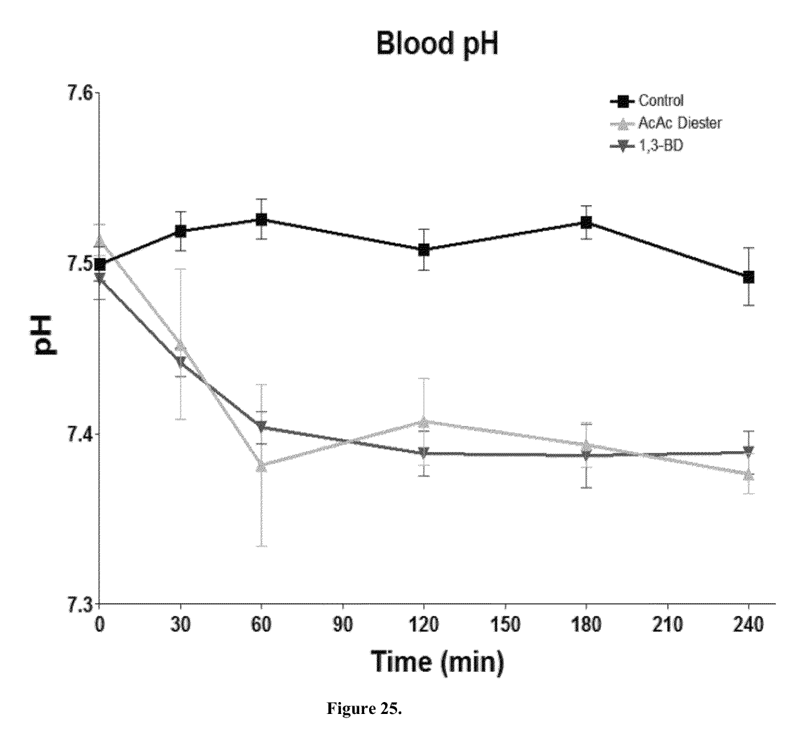

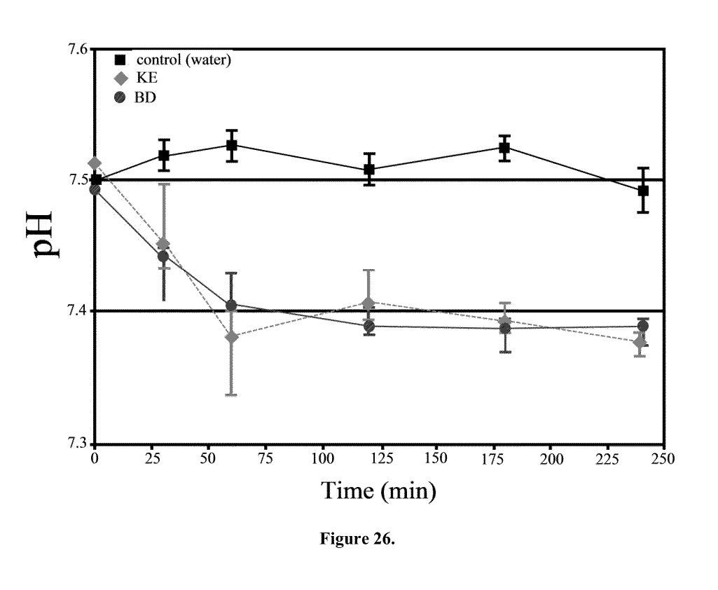

FIG. 25 shows increasing blood ketones with BD and BD-AcAc₂ causes a mild nonpathological acidosis. Mild acidosis is also common during the initial stages of the KD, and is typically attenuated with respiratory and renal compensation. Blood pH following KE or BD decreased compared to the control (pH˜7.5), by a mean of 0.05 after about 30 minutes and 0.1 after about one hour. No significant difference in pH was found between KE and BD treatment, seen in FIG. 26.

An unexpected finding was that KE caused a significant and sustained increase in blood pO₂ levels of approximately 30%. It's conceivable that these changes in PO₂ result from KE-induced redox alterations in the neural control of autonomic regulation, including cardiorespiratory function (Mulkey et al. 2003). Current studies are being done to determine the specific contribution of KE on brain O₂ consumption, ventilatory drive and cardiorespiratory modulation preceding CNS-OT.

One explanation for the mechanism by which KE delays CNS-OT is a shift in redox homeostasis, or a preservation of redox state during a hyperoxia-induced oxidative stress. This mechanism is plausible if one accepts the “free radical theory of CNS-OT”, which posits that the body's antioxidant defenses are overwhelmed by increased production of ROS (Gerschman et al. 1954). In support of this theory is the observation that brain and blood levels of ROS and reactive nitrogen species (RNS) increase just prior to HBO₂-induced seizures (Clark and Thom 1997; Demchenko et al. 2003). Previous research by the inventors has shown that superoxide production and neuronal excitability in the CA1 hippocampus is tightly coupled to tissue O₂ concentration ranging from 20-95% (D'Agostino et al. 2007). Considering the cellular and physiological effects of CNS-OT and the redox modulating effects of ketones (Maalouf et al. 2007; Veech 2004), it is not surprising that supra-physiological therapeutic ketosis significantly delays CNS-OT.

As reported previously, the anticonvulsant mechanism of therapeutic ketosis is largely unknown (Bough and Rho 2007). Therapeutic ketosis through fasting, calorie restriction and the KD activate numerous endogenous antioxidant pathways (Maalouf et al. 2009). These observations may explain how therapeutic ketosis, induced by fasting, protects against HBO₂-induced lipid peroxidation (Habib et al. 1990). Recently it has been shown that diet-induced ketogenesis improves mitochondrial redox state via activation of transcription factor Nrf2 (Milder et al. 2010), which is considered a master regulator of endogenous antioxidant regulation systems. Exogenous ketones also have direct antioxidant effects and protect against models of neurodegenerative disease (Maalouf et al. 2007). The metabolic shift in substrate utilization (from glucose to ketones) stabilizes synaptic function (Hartman et al. 2007), and activates signaling pathways associated with synaptic stability. Preliminary evidence suggests that an elevation of specific ketones (AcAc) may be responsible for stabilization of synapses. Ketones may prevent synaptic dysfunction by preserving brain metabolism during metabolic stress or oxidative stress from excess ROS production (Kim do et al. 2010; Veech 2004). This consistent with previous in vitro experiments, which showed that ketones significantly decrease superoxide production in primary neuronal cultures exposed to hyperoxia (D'Agostino et al. 2011).

The buffering systems that maintain redox homeostasis are highly compartmentalized with three major redox couples: GSH/GSSG, oxidized/reduced thioredoxin and cysteine/cystine. These redox couples control the equilibrium between oxidized and reduced states of cysteines and methionines. Importantly, the redox couples are not in equilibrium with each other and therefore can be considered as independent nodes of redox control (Jones, 2004). The oxidation state, affecting proteins with thiol/disulfide switches, can be altered by metabolic changes, environmental stressors and disease states. Although the intracellular GSH/GSSG redox state appears to most accurately reflect the tissue antioxidant defense capability, the extracellular Cys/CySS redox state is known to regulate cell functions (Hansen, 2006). Evidence suggests that therapeutic ketosis will influence extracellular redox state (Milder and Patel 2011; Veech 2004).

The neuroprotective effects of ketone bodies may be linked to their antioxidant effects. Glutamate-induced ROS production is inhibited by ketone bodies in primary cultures of rat neocortical neurons (Maalouf et al. 2007). Recently it's been shown that diet-induced ketogenesis improves mitochondrial redox state via the transcription factor Nrf2 (Milder and Patel 2011; Milder et al. 2010), which is considered the “hub” of endogenous antioxidant regulation. Ketone bodies also protect against cell death and impairment of long term potentiation after neocortical slices are exposed to hydrogen peroxide (Maalouf et al. 2009). In addition to effects on neurotransmission, ketones may prevent synaptic dysfunction by reducing ROS and preserving brain metabolism during metabolic or oxidative stress (Kim do et al. 2010; Veech 2004).

Data suggests preferential utilization of specific ketones for brain function confers neuroprotection against CNS-OT. Chavko et al (1999) demonstrated fasting (24 hrs) delays CNS-OT, but this effect was independent of blood glucose or elevation of BHB (via 1,3-butanediol injection). The present results support Chavko et al. and the lack of efficacy with BHB precursors (1,3-BD and 1,3-BD BHB ester). 1,3-BD AcAc monoester and 1,3-BD AcAc diester delays CNS-OT, but the mechanism is unknown, so it becomes essential to determine how ketogenesis affects markers of metabolic function and synaptic stability. The anticonvulsant effects of KE can be enhanced with chronic administration, due higher levels of ketones (primarily AcAc and acetone), and metabolic adaptation that involves upregulation of monocarboxylic acid transporters 1-4 (MCT 1-4) and activation of neuroprotective redox-sensitive metabolic signaling pathways.

Ketone bodies target a number of metabolic and neurophysiological signaling pathways (McNally and Hartman 2011), including reduced mitochondrial ROS production in response to an oxidative challenge (Kim do et al. 2010) and enhanced mitochondrial function (Veech et al. 2001). KE-induced neuroprotection is dependent on elevated ketones (AcAc, acetone), reduced oxidative stress and activation of neuroprotective pathways. Specific KE's confer protection against CNS-OT through multiple mechanisms involving enhanced brain metabolism and activation of neuroprotective redox-dependent signaling pathways. Neuroprotection against CNS-OT may require an elevation of ketone levels that mimics starvation (>3 mM), and that a significant rise in AcAc is essential.

### Example 5

In order to determine the anti-cancer effects of supplemental ketone therapy in the VM-M3 mouse model of metastatic cancer, R,S-1,3-butanediol diacetoacetate ester (KE) and 1,3-butanediol (BD) as sources of supplemental ketones for metabolic therapy, survival time, rate of tumor growth, body weight, blood glucose, and blood ketones was measured in mice with VM-M3 metastatic cancer treated with KE and BD administered with either standard or ketogenic diets.

Many cancers are unable to effectively utilize ketone bodies for energy (Maurer, et al. (2011) Differential utilization of ketone bodies by neurons and glioma cell lines: a rationale for ketogenic diet as experimental glioma therapy. BMC Cancer. 2011 Jul. 26; 11:315; Tisdale & Brennan (1983) Loss of acetoacetate coenzyme A transferase activity in tumours of peripheral tissues. British journal of cancer 47: 293-297; Magee, et al. (1979) The inhibition of malignant cell growth by ketone bodies. The Australian journal of experimental biology and medical science 57: 529-539). Furthermore, evidence suggests that ketones themselves possess inherent anti-cancer properties as βHB administration inhibits cancer cell proliferation and viability in vitro (Sawai, et al. (2004) Growth-inhibitory effects of the ketone body, monoacetoacetin, on human gastric cancer cells with succinyl-CoA: 3-oxoacid CoA-transferase (SCOT) deficiency. Anticancer research 24: 2213-2217; Magee, et al. (1979) The inhibition of malignant cell growth by ketone bodies. The Australian journal of experimental biology and medical science 57: 529-539). KDs are low carbohydrate, high fat diets that induce a modest elevation in blood ketone levels. R,S-1,3-butanediol-diacetoacetate ester (KE) is a non-ionized precursor to ketone bodies resulting in rapid elevation in ACA, and sustained elevation in βHB. 1,3-butanediol (BD) is a non-toxic food additive and hypoglycemic agent that is metabolized by liver to produce 3-hydroxybutyrate. Both are potential food sources of supplemental ketone bodies which significantly elevate blood ketone concentrations regardless of diet (Desrochers, et al. (1995) Metabolism of (R,S)-1,3-butanediol acetoacetate esters, potential parenteral and enteral nutrients in conscious pigs. The American journal of physiology 268: E660-667; Kies, et al. (1973) Utilization of 1,3-butanediol and nonspecific nitrogen in human adults. The Journal of nutrition 103: 1155-1163; Puchowicz, et al. (2000) Dog model of therapeutic ketosis induced by oral administration of R,S-1,3-butanediol diacetoacetate. The Journal of nutritional biochemistry 11: 281-287; Brunengraber (1997) Potential of ketone body esters for parenteral and oral nutrition. Nutrition 13: 233-235; Tobin, et al. (1975) Nutritional and metabolic studies in humans with 1,3-butanediol. Federation proceedings 34: 2171-2176). To investigate the anti-cancer potential of ketones in vivo, the effects of supplemental ketone administration were tested alone and combined with the KD on the VM-M3 mouse model of metastatic cancer.

Mice were separated into treatment groups, as provided in Table 3. On day 0 of the study, 1 million VM-M3/Fluc cells in 300 μL PBS were subcutaneously implanted into the abdomen of male, as described in the previous example, and randomly assigned to one of the five study groups.

Table 3 depicts the mouse feed groups for treatment

| Treatment group | Treatments (food and pressure treatment) |
|---|---|
| SD (Control) | Standard diet fed ad libitum |
| CR | Calorie Restricted diet |
| KE | Standard diet + 10% KE fed ad libitum |
| SDKE | Standard diet + 20% KE fed ad libitum |
| SDBD | Standard diet + 20% BD fed ad libitum |
| KDKE | KD-USF ketogenic diet food + 10% KE fed ad libitum |
| KDBD | KD-Solace ketogenic diet food + 20% BD fed |
| | ad libitum |

Two sources of supplemental ketones were used in this study: the R,S-1,3-butanediol-diacetoacetate ester (Ketone Ester, KE) and 1,3-butanediol (BD). The KE was synthesized (Savind Inc., Seymour Ill.) as previously described (D'Agostino et al., Therapeutic ketosis with ketone ester delays central nervous system oxygen toxicity seizures in rats. Am J Physiol Regul Integr Comp Physiol 2013, 304(10):R829-836) by transesterification of t-butylacetoacetate with R,S-1,3-butanediol (Savind Inc) and is a non-ionized, sodium-free, pH-neutral precursor of acetoacetate (ACA). The KE consists of two ACA molecules esterified to one molecule of 1,3-butanediol, an organic alcohol commonly used as a solvent in food flavoring agents. When ingested, gastric esterases rapidly cleave the KE to release two ACA molecules which are absorbed into circulation, rapidly elevating blood ketone concentration (Desrochers, et al. (1995) Metabolism of (R,S)-1,3-butanediol acetoacetate esters, potential parenteral and enteral nutrients in conscious pigs. The American journal of physiology 268: E660-667). The 1,3-butanediol molecule is absorbed and metabolized by the liver to produce βHB, providing a more sustained elevation of blood ketones. Administration of dietary BD is the second supplemental ketone source we will test, and it works to elevate ketone levels as previously described. The calorie restricted diet (CR) consisted of providing a daily food allotment of standard rodent chow restricted to 40% by weight compared to normal food consumption.

SDKE mice received standard rodent chow mixed at 20% KE and 1% saccharin by volume. SDBD mice received standard rodent chow mixed at 20% BD and 0.1 to 1% saccharin by volume. KDKE mice received KD-USF ketogenic diet food mixed at 20% KE and 1% saccharin by volume. Mice in the KDBD treatment group received KD-Solace ketogenic diet food mixed at 20% BD, 29% H₂O (to form a solid paste) and 0.1 to 1% saccharin by volume. See Table 4 for macronutrient information of diets and ketone supplements. Initial studies indicated that KD-Solace mixed with KE was severely unpalatable to the mice, so KD-USF mixed at 10% KE will be used for the KDKE group< since the KE was unpalatable to mice and was not consumed at 20% or when mixed with KD-Solace. Diets were continuously replaced to maintain freshness and allow mice to feed ad libitum.

Table 4 depicts the macronutrient information for SD, KD-Solace, KD-USF, BD,
and KE.

| | | Ketovolve | Custom | | |
|---|---|---|---|---|---|
| Macronutrient | Standard | (KD- | (KD- | Ketone | 1,3-Butane- |
| Information | Diet (SD) | Solace) | USF) | Ester (KE) | diol (BD) |
| % Cal from Fat | 6.2 | 89.2 | 77.1 | 0 | 0 |
| % Cal from Protein | 18.6 | 8.7 | 22.4 | 0 | 0 |
| % Cal from | 75.2 | 2.1 | 0.5 | 0 | 0 |
| Carbohydrate | | | | | |
| Caloric Density | 3.1 Kcal/g | 7.12 Kcal/g | 4.7 Kcal/g | 5.58 Kcal/g | 4 Kcal/g |

On the day of tumor inoculation, mice were randomly assigned to a treatment group. Control mice received standard rodent chow fed ad libitum. Mice receiving ketone supplementation diet therapy were administered their respective diet fed ad libitum in lieu of standard rodent chow. Saccharin was added to increase palatability and does not have a measurable effect on metabolism. Supplemental ketones may be unpalatable to the mice causing the mice to self-calorie restrict (Kashiwaya, et al. (2010) A ketone ester diet increases brain malonyl-CoA and Uncoupling proteins 4 and 5 while decreasing food intake in the normal Wistar Rat. The Journal of biological chemistry 285: 25950-25956).

Blood was collected from the study animals every 7 days. Blood glucose and βHB concentrations were measured using a commercially available Glucose and Ketone (βHB) Monitoring System (Nova Biomedical and Abbott Laboratories). Mice were weighed twice weekly for the duration of the study using the AWS-1Kg Portable Digital Scale (AWS). Blood and weight measurements were taken at the same time of day each week to control for normal fluctuations in feeding or circadian metabolic changes. Studies will focus on health and behavior of the animals on a daily basis. Survival time was measured as the time in days from cancer cell inoculation to presentation of defined criteria (diminished response to stimuli, loss of grooming or feeding behavior, lethargy, severe ascites, or failure to thrive). At that time, mice were humanely euthanized by CO₂ asphyxiation and survival time noted.

It was expected that supplemental ketone administration will increase survival time, slow tumor growth rate, decrease blood glucose, and elevate blood ketones in VM-M3 mice with metastatic cancer compared to control animals. Since the KE supplies more ketones to the tissues than BD, and ketones inhibit cancer cell proliferation in vitro (Sawai, et al. (2004) Growth-inhibitory effects of the ketone body, monoacetoacetin, on human gastric cancer cells with succinyl-CoA: 3-oxoacid CoA-transferase (SCOT) deficiency. Anticancer research 24: 2213-2217; Magee, et al. (1979) The inhibition of malignant cell growth by ketone bodies. The Australian journal of experimental biology and medical science 57: 529-539), it was expected that the anti-cancer effects of the supplemental ketone administration would be greater in KE-fed mice. Further, since carbohydrate restriction decreases blood glucose which cancer cells rely on for energy, combining KE or BD with a ketogenic diet was expected to be more effective than when combined with standard diet.

Cell proliferation rate was measured using the MTT Cell Proliferation Assay (ATCC). Cells were plated onto a 96 well plate and grown to desired density. Cells were treated for 72 hrs with low (5 mM) glucose, high (15 mM) glucose, or 5 mM βHB with or without HBO2T (100% 02, 2.5 ATA absolute, for 90 min). In proliferating cells, MTT is reduced to purple formazan which absorbs light at 490-520 nm and whose excitation can be measured using standard fluorescent microscopy and spectrophotometry. Rapidly dividing cells reduce MTT at very high rates, indicating their rate of proliferation. Cell proliferation can also be measured with Ki67 immunohistochemistry staining, cell viability can also be evaluated with the LDH Cytotoxicity Assay (Cayman Chemical).

Cell viability was measured using the LIVE/DEAD Viability/Cytotoxicity Kit for Mammalian Cells (Invitrogen). Cells were grown to desired density on a coverslip and washed with Dulbecco's phosphate-buffered saline (D-PBS). Cells were treated for 72 hrs with low (5 mM) glucose, high (15 mM) glucose, or 5 mM βHB with or without HBO2T (100% 02, 2.5 ATA absolute, for 90 min). The two-color fluorescence assay contains two probes which specifically label live or dead cells. Live cells possess ubiquitous intracellular esterases which cleave the non-fluorescent calcein AM into the highly fluorescent calcein. Calcein produces an intense green fluorescence with an excitation/emission of 495/515 nm. Ethidium homodimer-1 (Ethd-1) enters cells with damaged membranes and binds to nucleic acid. Ethd-1 bound to DNA produces a red fluorescence in dead cells with an excitation/emission of 495/635 nm. Live and dead cells were identified and quantified using standard fluorescent microscopy.

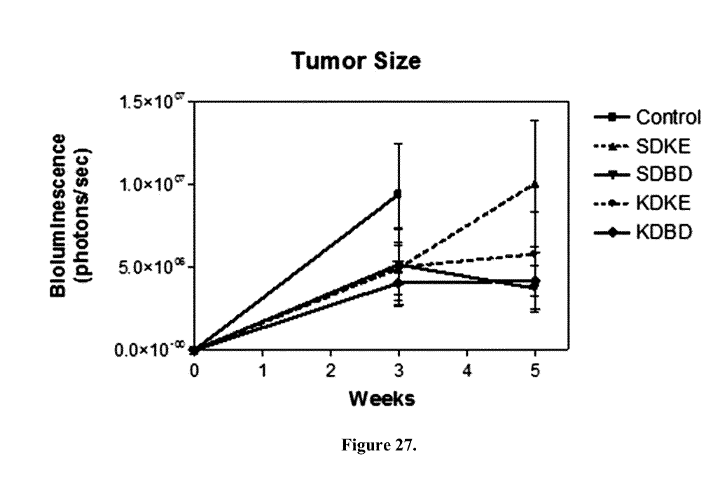

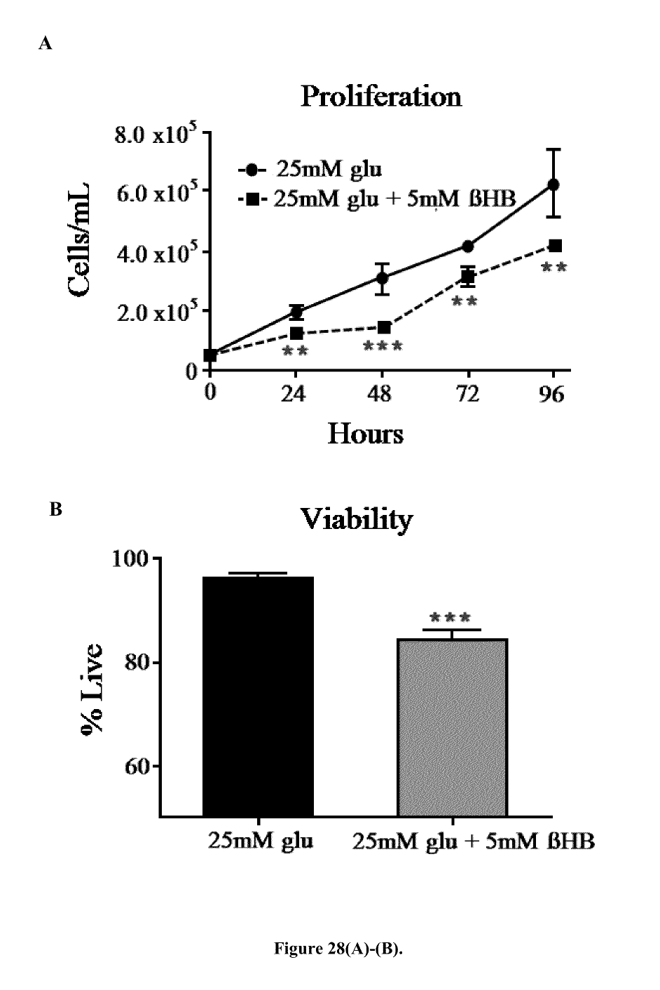

Animals receiving supplemental KD exhibited reduced tumor growth, seen in FIG. 27. Proliferation was significantly decreased in VM-M3 cells grown in 5 mM βHB supplemented control media compared to VM-M3 cells grown in control media at 24, 48, 72, and 96 hrs (**p<0.01, ***p<0.001; One-Way ANOVA; FIG. 28(A). Viability was significantly decreased by 12.1% in VM-M3 cells treated for 24 hrs with 5 mM βHB supplemented control media compared to VM-M3 cells grown in control media (***p<0.001; Two-tailed student's t-test; FIG. 28(B).

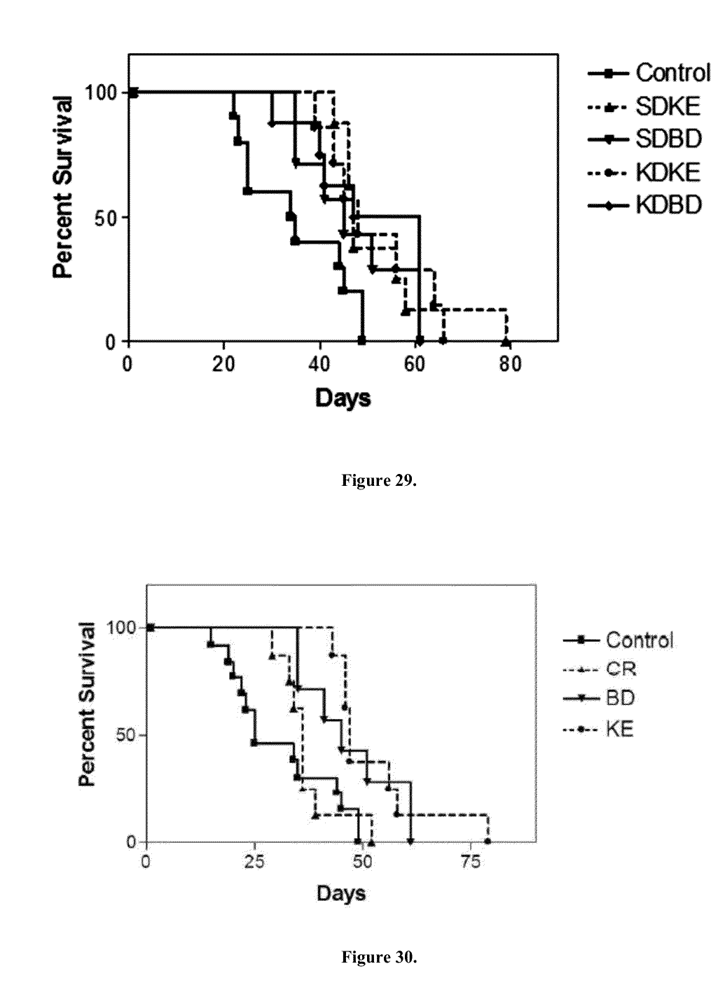

Animal survival was analyzed with the Kaplan-Meier and Logrank Tests for survival distribution. Mean survival and cell viability were analyzed by two-tailed student's t-tests. Supplemental KD increased mean survival, seen in FIG. 29 and Table 5. BD and KE treated mice demonstrated a significantly prolonged survival curve by LogRank Test for survival distribution compared to control animals (*p=0.02 and **p=0.01, respectively, FIG. 30). BD and KE treated mice also showed a significant increase in mean survival time compared to control animals, as seen in Table 5 (Two-tailed student's t-test; *p<0.05 and ***p<0.001, respectively). While previous reports have demonstrated that CR increases survival time in various animal cancer models, in this study, CR treated mice did not show statistically different survival time, as compared to controls (Logrank Test for survival distribution and Two-tailed student's t-test; p>0.05). Control (SD) mice lived an average of 31.2 days while CR mice had a non-statistically significant different mean survival time of 36.9 days (p>0.05; Two-tailed student's t-test; FIG. 30. BD treatment increased mean survival time by approximately 16 days (51%), and KE treatment increased mean survival time by approximately 22 days (69%) compared to controls (*p<0.05 and ***p<0.001, respectively; Two-tailed student's t-test), as seen in Table 5.

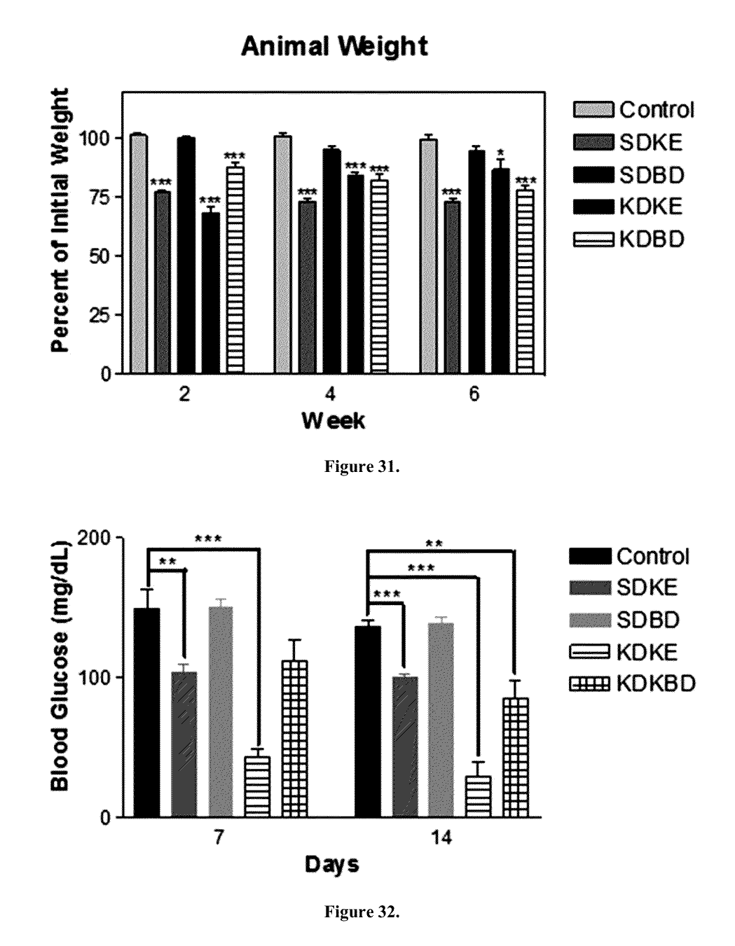

Supplementation of food with 20% KE (SDKE) or KD-USF ketogenic diet food mixed with 20% KE (KDKE) significantly reduced mouse weight at week 2, with KDKE mice normalizing slightly in weeks 4 and 6, as seen in FIG. 31. KD-Solace ketogenic diet food with 20% BD (KDBD) also exhibited decreased animal weight compared to control and rodent chow mixed with 20% BD (SDBD), though less dramatic than SDKE, as seen in FIG. 31. Blood glucose levels mirrored animal weight correlations, with SDKE, KDKE, and KDBD showing reduced glucose levels compared to controls, as seen in FIG. 32.

Table 5 shows the supplemental ketogenic diet increased survival time in mice with systemic metastatic cancer. The treatment cohort group and median survival times are shown.

| | | | % increase in |
|---|---|---|---|
| Treatment | Cohort size (N) | Mean survival (days) | survival time |
| control (SD) | 13 | 31.2 | — |
| CR | 8 | 36.9 | 18.3 |
| KE | 8 | 52.8 | 69.2* |
| SDKE | 8 | 52.8 | 69.2* |
| SDBD | 7 | 47 | 50.6* |
| KDKE | 7 | 51.6 | 65.4* |
| KDBD | 8 | 50.3 | 61.2* |

*p < 0.05
SDBD showed no body weight loss, increase in survival attributed to ketone supplement, not CR.

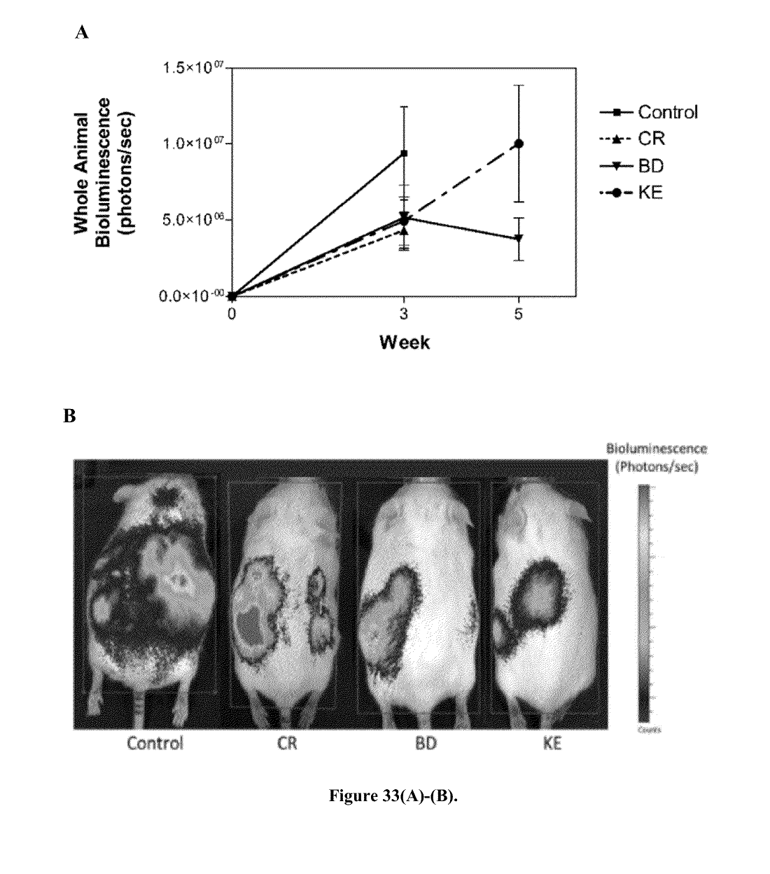

Tumor progression was measured using bioluminescence on the Xenogen IVIS Lumina cooled CCD camera (Caliper LS, Hopkinton, Mass.), seen in FIG. 33(B). Bioluminescent signal of the luciferase-tagged cancer was acquired with the Living Image® software (Caliper LS). Mice received an i.p. injection of 50 mg/kg D-Luciferin (Caliper LS) 15 minutes prior to imaging. Bioluminescent signal was obtained using the IVIS Lumina cooled CCD camera system with a 1 sec exposure time. Whole animal bioluminescent signal was measured in photons/sec once a week as an indicator of metastatic tumor size and spread. As seen in FIG. 33(A), KE treatment drastically reduced tumor progression, with BD treatment also showing profound effect. While previous reports have demonstrated that CR increases survival time in various animal cancer models, in this study, CR treated mice exhibited a trend of increased latency to disease progression measured as tumor bioluminescence, as seen in FIG. 33(B).

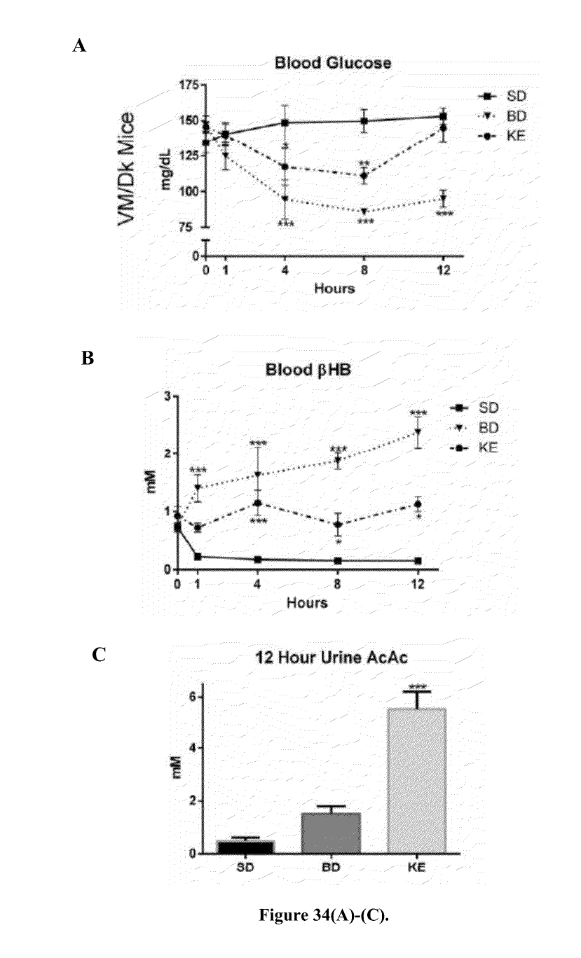

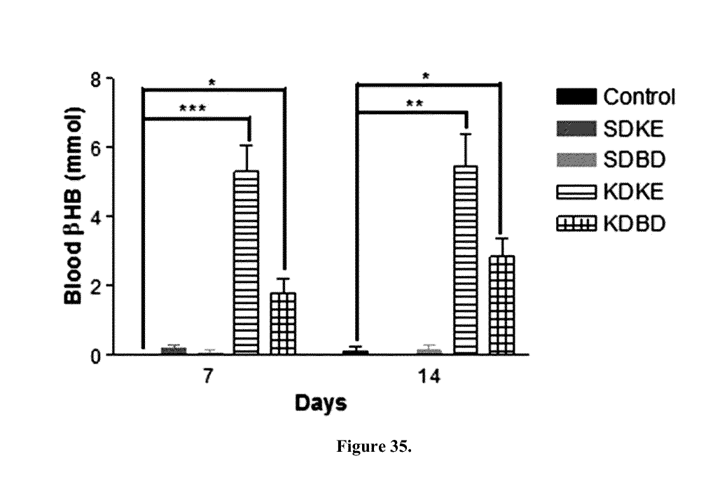

Standard high carbohydrate rodent chow with ketone supplementation significantly lowered blood glucose for 8 hours and 4 hours, in BD and KE groups, respectively (p<0.05; Two-Way ANOVA; FIG. 34(A). BD significantly elevated βHB levels after 1 hour which further increased in the next 11 hours, while KE caused significant βHB elevation only after 4 hours and remained at a similar level after 12 hours (p<0.05; Two-Way ANOVA; FIG. 34(B). BD only elevated βHB while KE elevated both βHB and AcAc (p<0.05; Two-Way ANOVA for βHB and One-Way ANOVA for AcAc; FIG. 34 (B, (C). Unlike the KD treatments disclosed above, ketone supplementation resulted in significantly higher ketone levels in KDKE and KDBD, at both 7 and 14 days, as seen in FIG. 35.

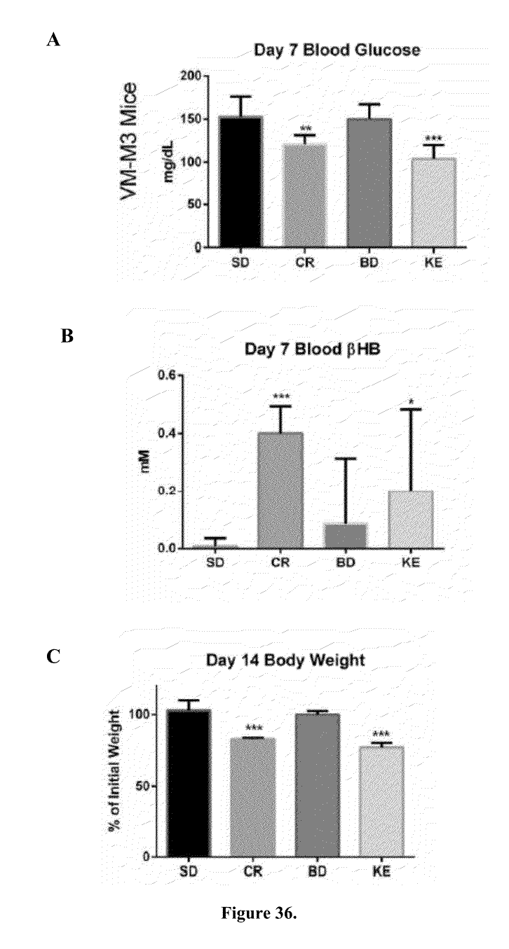

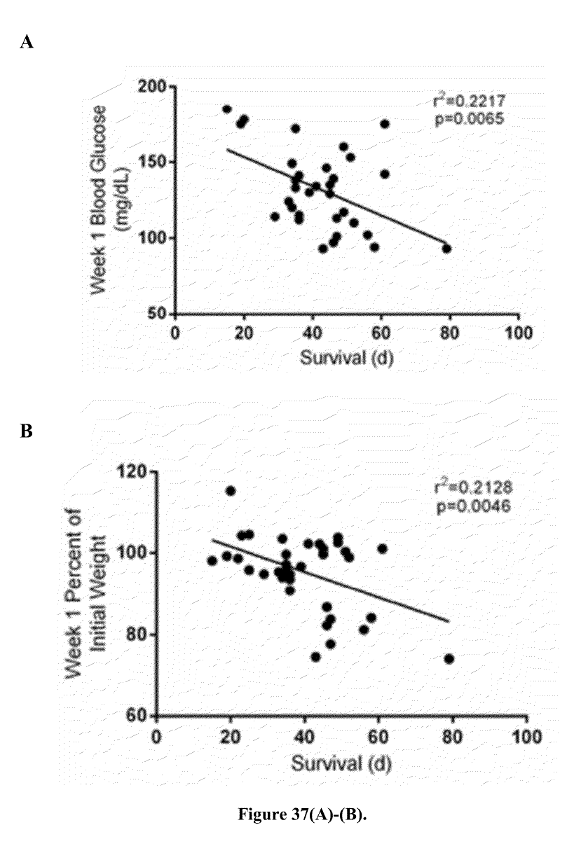

Acute ketosis with supplementation in healthy VM/Dk mice. Blood and weight measures in VM-M3 survival study mice. Initial blood glucose, βHB, and body weights were similar between groups (data not shown). Chronic ketone supplementation at day 7 resulted in lower blood glucose and elevated blood βHB in CR and KE treated animals compared to controls (p<0.05; One-Way ANOVA; FIG. 36 (A), (B). By day 14, CR and KE treated mice lost approximately 20% of their initial body weight (p<0.001; One-Way ANOVA), as seen in FIG. 36(C) and maintained that weight loss for the duration of the study. Day 7 blood glucose and body weight change were significantly correlated to survival (p=0.0065 and p=0.0046, respectively; Linear Regression Analysis), as seen in FIGS. 37(A) and (B).

On day 21 of the study, mice were euthanized by CO₂ asphyxiation and brain, heart, lungs, liver, kidneys, spleen, intestine, and samples of adipose tissue and skeletal muscle will be surgically removed Immediately following tissue extraction, organs were incubated in 300 μg/mL D-Luciferin in PBS for 5 min. Bioluminescence of the individual organs were imaged using a 1 second exposure time on the Xenogen IVIS Lumina cooled CCD camera (Caliper LS, Hopkinton, Mass.). Bioluminescent signal of the luciferase-tagged cancer was acquired with the Living Image® software (Caliper LS). Mice received an i.p. injection of 50 mg/kg D-Luciferin (Caliper LS) 15 minutes prior to imaging. Bioluminescent signal was obtained using the IVIS Lumina cooled CCD camera system with 1 sec exposure time. Whole animal bioluminescent signal was measured in photons/sec once a week as an indicator of metastatic tumor size and spread, through measuring intensity of bioluminescent signal (photon count) produced by the organs. Tissues were immediately flash frozen in liquid nitrogen to preserve viability for vessel density and protein expression studies.

Flash frozen hepatic tumor tissue were embedded in OCT compound and cut with a cryostat to produce 10 μm tissue sections for analysis of blood vessel density. Sections were mounted onto histological slides and stained with anti-mouse von Willibrand factor (vWf), an endothelial cell-specific glycoprotein, staining blood vessels brown. Slides were visualized and blood vessel density will be determined by counting the number of vWf⁺ blood vessels within a region of interest in a blinded manner.

Lung tumor protein expression of Insulin-like Growth Factor-1 (IGF-1), Activated Akt, Activated Mammalian Target of Rapamycin (mTOR), Hypoxia-Inducible Factor-1α (HIF-1α), and Vascular Endothelial Growth Factor (VEGF) were measured by standard western blot techniques using Anti-IGF-1, Anti-Phospho-Akt, Anti-Phospho-mTOR, Anti-HIF-1α, and Anti-VEGF antibodies (Sigma-Aldrich). Protein density will be determined using the GE Typhoon 9400 Imager with ImageQuant TL software (GE Life Sciences).

It was concluded that supplemental ketone administration confers anti-cancer effects when delivered with either standard or ketogenic diet. SDBD mice did not show significant loss of weight but still had effects, indicating that ketones did induce significant results

### Example 6

To determine the anti-cancer effects of KD metabolic therapy and HBO2T, survival time, rate of tumor growth, body weight, blood glucose, and blood ketones were measured in mice with VM-M3 metastatic cancer treated with KD, HBO2T, or combined KD+HBO2T.

KD is useful as a metabolic therapy for cancer by reducing availability of glucose, the main energy substrate for tumors, and inhibiting several oncogene pathways such as IGF-1, MYC, mTOR, and Ras. HBO2T increases oxygen saturation inside tissues, reversing the cancer-promoting effects of tumor-hypoxia and enhancing ROS production which can induce cell death (D'Agostino, et al. (2009) Acute hyperoxia increases lipid peroxidation and induces plasma membrane blebbing in human U87 glioblastoma cells. Neuroscience 159: 1011-1033). While these therapies have been evaluated separately, the overlapping mechanisms mediating their efficacy can be significantly enhanced by combining the treatments. Furthermore, even though metastasis is responsible for 90% of cancer deaths, few studies have evaluated metabolic therapy or HBO2T as a treatment for metastatic cancer. Therefore, the individual and combined anti-cancer effects of the ketogenic diet and HBO2T were evaluated in the VM-M3 mouse model of metastatic cancer (Huysentruyt, et al. (2008) Metastatic cancer cells with macrophage properties: evidence from a new murine tumor model. International journal of cancer Journal international du cancer 123: 73-84).

VM-M3/Fluc cells (T. Seyfried; Boston College) were obtained from a spontaneous tumor in a VM/Dk inbred mouse and adapted to cell culture (Huysentruyt et al., Metastatic cancer cells with macrophage properties: evidence from a new murine tumor model. Intl J Can. 2008, 123(1):73-84). VM-M3/Fluc cells were transfected with the firefly luciferase gene which produces a bioluminescent product in the presence of the enzymatic substrate luciferin (Shelton, et al. (2010) A novel pre-clinical in vivo mouse model for malignant brain tumor growth and invasion. Journal of neuro-oncology 99: 165-176). Bioluminescence can be detected and measured with the Xenogen IVIS Lumina System (Caliper LS). Intensity of bioluminescent signaling (photon count) is directly correlated to the number of luciferase-tagged cells within the animal (Kim, et al. (2010) Non-invasive detection of a small number of bioluminescent cancer cells in vivo. PloS one 5: e9364; Lim, et al. (2009) In vivo bioluminescent imaging of mammary tumors using IVIS spectrum. Journal of visualized experiments: JoVE) and is a well-accepted method of measuring tumor size in animals with luciferase-expressing tumors (Lyons (2005) Advances in imaging mouse tumour models in vivo. The Journal of pathology 205: 194-205; Close, et al. (2011) In vivo bioluminescent imaging (BLI): noninvasive visualization and interrogation of biological processes in living animals. Sensors (Basel, Switzerland) 11: 180-206). Mice received an i.p. injection of 50 mg/kg D-Luciferin 15 minutes prior to in vivo imaging. Bioluminescent signal was recorded using a 1 second exposure time on the IVIS Lumina cooled CCD camera. Progression of the metastatic cancer was measured by tracking the bioluminescent signal of the whole animal over time. Tumor bioluminescence was measured once weekly for the duration of the study.

Adult male mice (2-4 months of age) were separated into treatment groups, as provided in Table 6. On day 0 of the study, 1 million VM-M3/Fluc cells in 300 μL PBS were subcutaneously implanted into the abdomen of male, 10-18 week old VM/Dk mice using a 27 g needle. With the VM-M3 model, an adipose tumor quickly appeared following inoculation and rapidly metastasized to most major organs, including brain, lungs, liver, spleen, kidneys, and bone marrow (Huysentruyt, et al. (2008) Metastatic cancer cells with macrophage properties: evidence from a new murine tumor model. International journal of cancer Journal international du cancer 123: 73-84). On the day of tumor inoculation, mice will be randomly assigned to one of five study groups: Control, KD-Solace, KD-USF, SD+HBO2T or KD+HBO2T. On the day of tumor inoculation, mice were randomly assigned to a treatment group<shown in Table 6.

Table 6 shows mouse feed groups for treatment

| Treatment group | Treatments (food and pressure treatment) |
|---|---|
| Control | Standard diet fed ad libitum; ambient pressure |
| KD-Solace | Commercially available (Ketovolve, Solace Nutrition) |
| | ketogenic food fed ad libitum; ambient pressure |
| KD-USF | Teklad Custom Research Ketogenic diet designed by |
| | researchers (Harlan Laboratories) fed ad libitum; |
| | ambient pressure |
| SD + HBO2T | Standard diet fed ad libitum + HBO2T |
| KD + HBO2T | KD-Solace food fed ad libitum + HBO2T |

Control mice were fed standard rodent chow (2018 Teklad Global 18% Protein Rodent Diet, Harlan Laboratories) fed ad libitum. Mice on a diet therapy received their respective diet fed ad libitum in lieu of standard rodent chow. Mice in the KD-Solace treatment group received KD-Solace (Solace Nutrition) ketogenic diet food, mixed 1:1 with H₂O to form a paste. Mice in the KD-USF treatment group received a Teklad Custom Research Diet (Harlan Laboratories) designed by the researchers. The macronutrient information of the diets used in this study is provided in Table 7. The macronutrient ratio of the custom designed KD-USF diet is similar to ketogenic diets with very low carbohydrate (VLC), containing a high percentage of MCT oil (30-40%) and high protein (22%). The KD-USF diet is notably more palatable to the mice. Diets will be continuously replaced to maintain freshness and allow mice to feed ad libitum.

Table 7 depicts macronutrient information for SD, KD-Solace,
and KD-USF.

| | Standard Diet | Ketovolve | Custom |
|---|---|---|---|
| Macronutrient Information | (SD) | KD-Solace | KD-USF |
| % Cal from Fat | 6.2 | 89.2 | 77.1 |
| % Cal from Protein | 18.6 | 8.7 | 22.4 |
| % Cal from Carbohydrate | 75.2 | 2.1 | 0.5 |
| Caloric Density | 3.1 Kcal/g | 7.12 Kcal/g | 4.7 Kcal/g |

Mice in the SD+HBO2T and KD+HBO2T treatment groups received HBO2T (100% oxygen) at 2.5 ATA absolute (1.5 ATA gauge) for 90 minutes three times a week (M,W,F) pressurized in a hyperbaric chamber.

Blood was collected from the study animals every 7 days. Blood glucose and βHB concentrations were measured using a commercially available Glucose and Ketone (βHB) Monitoring System (Nova Biomedical and Abbott Laboratories). Mice were weighed twice weekly for the duration of the study using the AWS-1Kg Portable Digital Scale (AWS). Blood and weight measurements were taken at the same time of day each week to control for normal fluctuations in feeding or circadian metabolic changes. Studies will focus on health and behavior of the animals on a daily basis. Survival time was measured as the time in days from cancer cell inoculation to presentation of defined criteria (diminished response to stimuli, loss of grooming or feeding behavior, lethargy, severe ascites, or failure to thrive). At that time, mice were humanely euthanized by CO₂ asphyxiation and survival time noted.

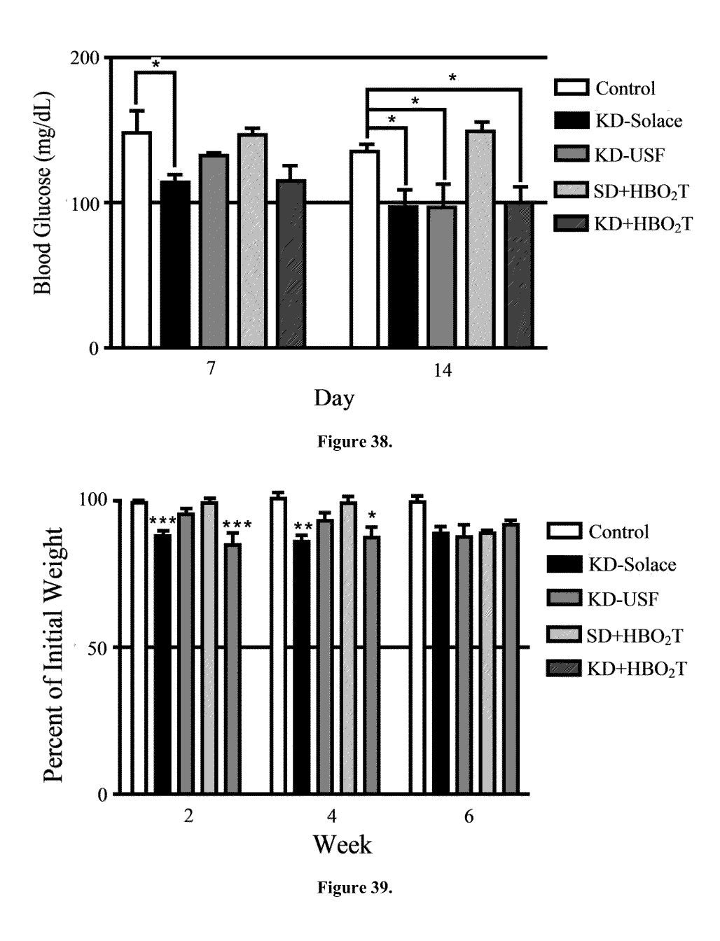

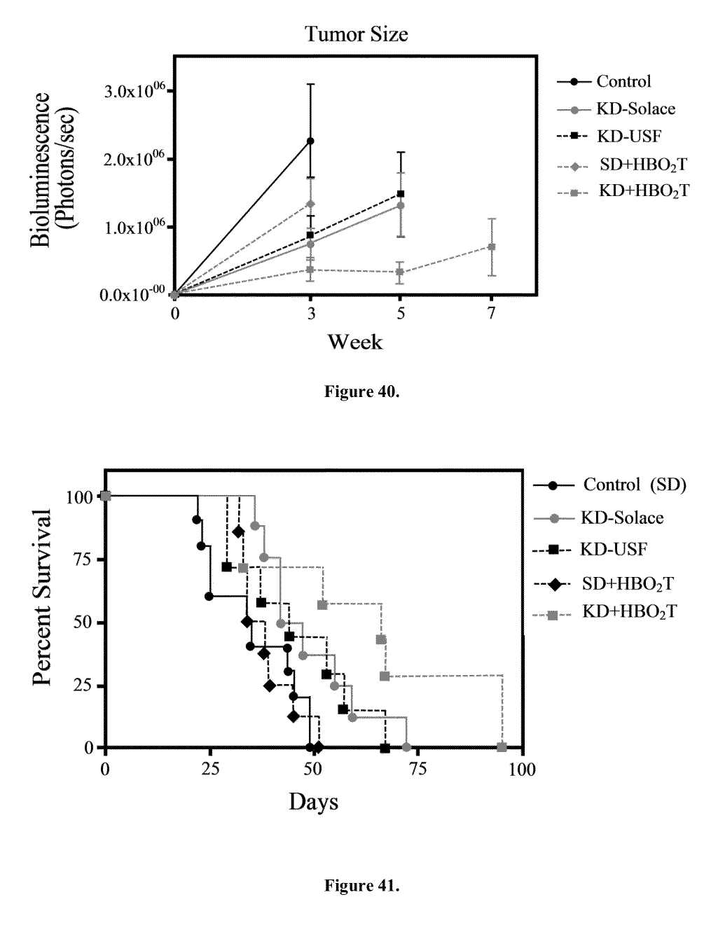

Animals receiving KD had lower glucose, and some body weight loss compared to controls, as seen in FIGS. 38 and 39, respectively. Due to the previously reported anti-cancer effects of the KD and HBO2T, animals in these treatment groups exhibited reduced tumor growth and prolonged survival relative to control, seen in FIGS. 40 and 41, respectively. Since metabolic therapy and HBO2T target overlapping pathways, combining the KD with HBO2T were expected to result in a synergistic decrease in tumor growth rate and increase in survival, as seen in Table 8.

Table 8 depicts the treatment group cohort size and median survival times.
KD-Solace mice exhibited a 34% increase in mean survival time compared
to controls (p = 0.0249); KD-HBO2T mice exhibited an 80% increase
in mean survival time compared to controls (p = 0.0082).

| | | Mean survival | % increase in survival |
|---|---|---|---|
| Treatment | Cohort size (N) | (days) | time |
| control (SD) | 10 | 35.1 | — |
| KD-Solace | 8 | 48.9 | 39.3* |
| KD-USF | 7 | 45.1 | 28.5 |
| SD + HBO2T | 8 | 38.8 | 10.5 |
| KD + HBO2T | 11 | 55.5 | 80** |

*p < 0.05
**p < 0.001

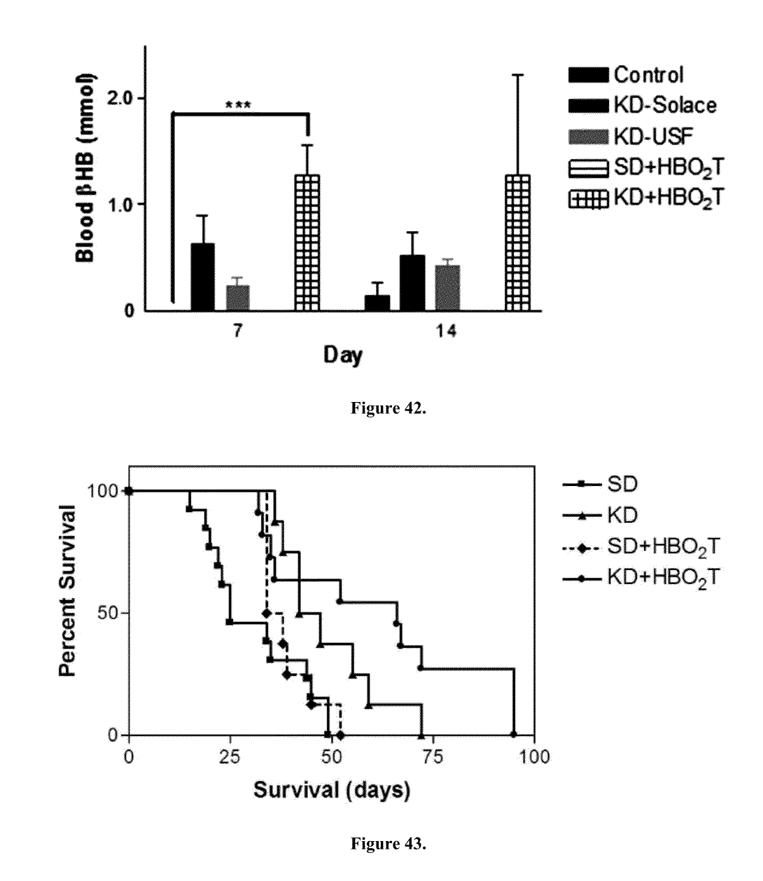

While it was expected that KD treatment would result in higher ketones, results showed a transitory increase in blood ketones, with a non-significant difference at 14 days, as seen in FIG. 42.

Most studies examining the effects of HBO2T on cancer have focused on solid, primary tumors. Since hypoxia is most prevalent inside large tumors, it is possible that HBO2T will not be as effective a treatment for metastatic disease compared to solid tumors. This would not be a problem but rather evidence providing insight into the specific conditions in which HBO2T might serve as an effective anti-cancer therapy. When given as individual therapies, the KD but not HBO2T elicited anti-cancer effects in mice with systemic metastatic cancer. However, combining the KD with HBO2T elicited profound, supra-additive anti-cancer effects, indicating a synergistic mechanism of action.

### Example 7

To determine if supplemental ketone metabolic therapy and HBO2T work synergistically to inhibit the progression of metastatic cancer, survival time, rate of tumor growth, body weight, blood glucose, and blood ketones were measured in mice receiving supplemental ketones with HBO2T. To further assess this combination therapy, the extent of organ metastasis in time-matched tumors, blood vessel density, and protein expression of important cancer signaling molecules were examined in VM-M3 mouse tumors ex vivo following treatment with the proposed therapies.

Data indicate that individually, the KD and ketone supplementation inhibit cancer progression, and that combining the KD with HBO2T had profound synergistic anti-cancer effects. Throughout the study, the KDKE mice exhibited the lowest blood glucose and highest blood ketone levels of the treatment groups. As previously discussed, lowering blood glucose and elevating blood ketones work through several mechanisms to inhibit cancer growth. Furthermore, KDKE therapy resulted in greater anti-cancer effects than the KD alone. Since KD combined with HBO2T induced supra-additive anti-cancer effects and KDKE therapy was more efficacious than KD-alone, combining the KDKE diet therapy with HBO2T elicited an even greater response. To determine the efficacy of these combined treatments, the survival, rate of tumor growth, body weights, blood glucose, and blood ketones was studied in VM-M3 mice receiving KDKE+HBO2T therapy. To further investigate the synergistic effects of KD, supplemental ketones, and HBO2T treatment on metastatic cancer, the extent of organ metastasis, blood vessel density, and protein expression of important signaling molecules in tumors ex vivo was measured from VM-M3 mice receiving KD+HBO2T, KDKE, and KDKE+HBO2T therapies compared to control animals.

Combining the KD with HBO2T or KE confers potent anti-cancer effects in our model; therefore, KDKE+HBO2T treated mice should demonstrate even greater efficacy with increased survival time and decreased tumor growth rate. All treated mice should demonstrate reduced organ metastasis compared to control animals although it is unclear if this will be due to inhibition of primary tumor growth or effects on the metastatic process itself. Animals treated with HBO2T will likely demonstrate significantly less tumor vasculature, as hyperoxia inhibits many angiogenic factors known to be overactive in cancer. The proposed signaling molecules should be elevated in relation to the hypoxic and glycolytic phenotype of cancer through mechanisms previously discussed. Therefore, we expect the expression of these molecules to be decreased in animals treated with metabolic therapy and HBO2T compared to controls.

To gain a greater understanding of the mechanisms of the anti-cancer effects of these treatments, cell proliferation, viability, reactive oxygen species (ROS) production, and cell morphology of VM-M3 cells in vitro following exposure to low and high glucose, ketones, and HBO2T were measured. The rate of cell proliferation, cell viability, production, and membrane lipid peroxidation induced-changes in cell morphology (indicative of oxidative stress) of VM-M3/Fluc cells in response to treatment with low (3 mM) glucose, high (15 mM) glucose, 5 mM βHB, and HBO2T (100% 02, 2.5 ATA) compared to control, non-treated cells. Cells were treated with low glucose (5 mM); high glucose (15 mM); 5 mM βHB; with/without hyperbaric oxygen therapy (100% 02, 2.5 ATA).

VM-M3/Fluc cells transduced with a lentivirus vector containing the firefly luciferase gene, as discussed in the previous examples, were cultured in Eagle's Minimum Essential Medium with 2 mM L-glutamine, 10% fetal bovine serum, 1% penicillin-streptomycin, and 10 mM D-glucose. Cells will be maintained in a CO₂ incubator at 37° C. in 95% air and 5% CO₂. Cells receiving HBO2T were placed in a standard hyperbaric chamber and pressurized to 2.5 ATA absolute with 100% O₂ for 90 min. 5 mM HEPES is added to maintain CO₂ concentrations while in HBO2T chamber.

Forty VM/Dk adult male mice (10-18 weeks of age) were s.c. implanted into the abdomen on day 0 with VM-M3/Fluc cells (1 million cells in 300 mL PBS) using a 27 gage needle. Inoculation results in rapid and systemic metastasis to most major organs, namely liver, kidneys, spleen, lungs, and brain as previously described (Huysentruyt, et al. (2008) Metastatic cancer cells with macrophage properties: evidence from a new murine tumor model. International journal of cancer Journal international du cancer 123: 73-84).

On the day of tumor inoculation, mice were randomly assigned to one of four groups: SD (Control); SD+HBO2T; KD; or KD+HBO2T. Mice in the SD group were fed standard rodent chow (2018 Teklad Global 18% Protein Rodent Diet, Harlan) ad libitum. Mice in the KD group received KD-Solace ketogenic diet ad libitum. KD-Solace is a commercially available ketogenic diet powder (KetoGen, Solace Nutrition) and was mixed 1:1 with H₂O to form a solid paste. Macronutrient information for SD and KD-Solace are shown in Table 9. Diets were continuously replaced every other day to maintain freshness and allow mice to feed ad libitum.

Table 9 depicts macronutrient information for SD, KD-Solace in this
example.

| | Standard Diet | Ketovolve |
|---|---|---|
| Macronutrient Information | (SD) | KD-Solace |
| % Cal from Fat | 18.0 | 89.2 |
| % Cal from Protein | 1240 | 8.7 |
| % Cal from Carbohydrate | 58.0 | 2.1 |
| Caloric Density | 3.1 Kcal/g | 7.12 Kcal/g |

Mice undergoing HBO2T received 100% O₂ for 90 minutes at 1.5 ATM gauge (2.5 ATM absolute) three times per week (M, W, F) in a hyperbaric chamber (Model 1300B, Sechrist Industries, Anaheim, Calif.).

Bioluminescent signal was tracked as a measure of tumor size throughout the study. Tumor growth was monitored as a measure of bioluminescent signaling using the Xenogen IVIS Lumina system (Caliper LS, Hopkinton, Mass.). Data acquisition and analysis was performed using the Living ImageH software (Caliper LS). Approximately 15 minutes prior to in vivo imaging, the mice received an i.p. injection of D-Luciferin (50 mg/kg) (Caliper LS). Bioluminescent signal was obtained using the IVIS Lumina cooled CCD camera system with a 1 sec exposure time. As only the cancer cells contained the luciferase gene, bioluminescent signal (photons/sec) of the whole animal was measured and tracked over time as an indicator of metastatic tumor size and spread.

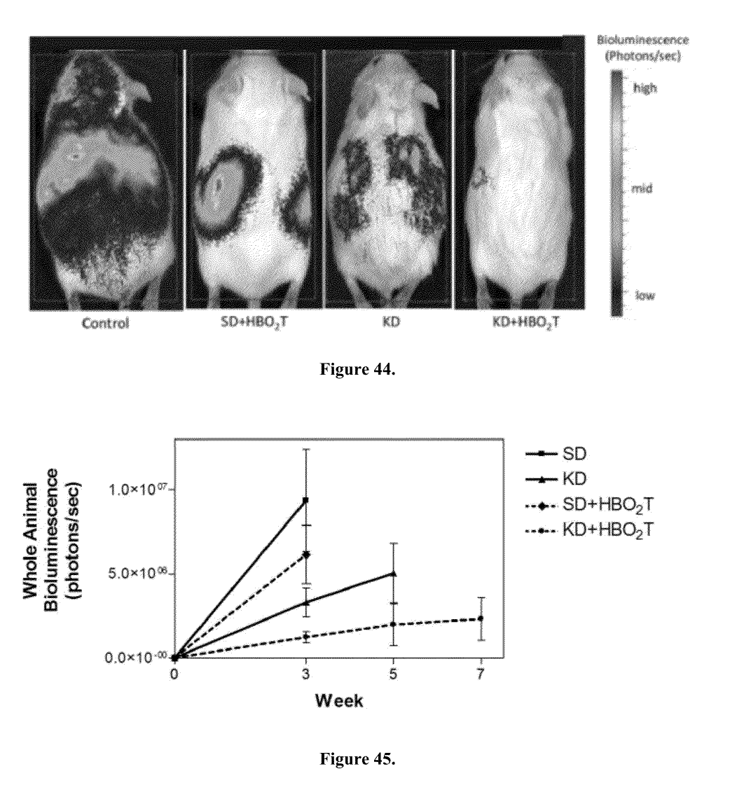

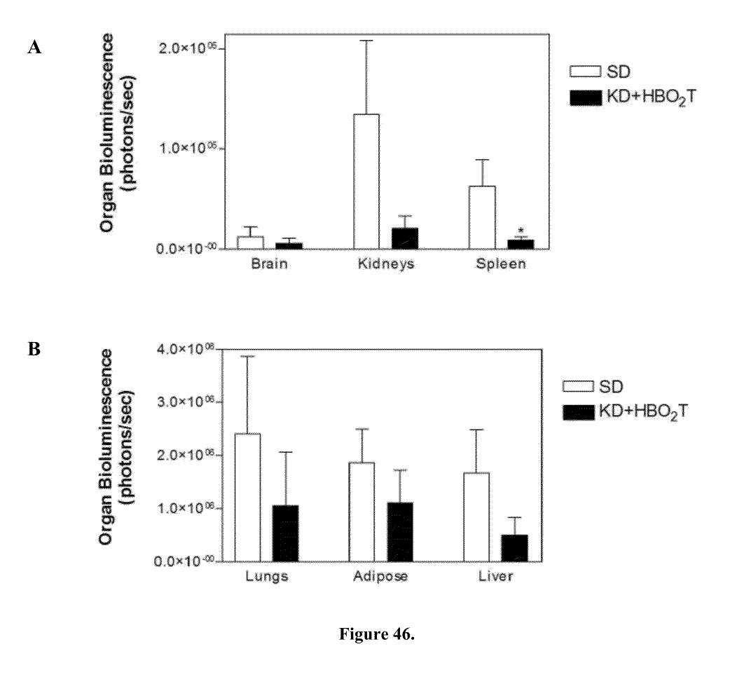

Animals receiving the KD alone or in combination with HBO2T demonstrated a notable trend of slower tumor growth over time. This trend was more pronounced in KD+HBO2T mice and reflected the increase in survival time seen in these animals, as seen in FIGS. 43 through 46(B), and Table 10. The difference in mean tumor size between KD+HBO2T and control animals at week 3 was statistically significant (p=0.0062), seen in FIG. 45. Day 21 ex vivo organ bioluminescence of KD+HBO2T mice demonstrated a trend of reduced metastatic tumors in animals compared to the SD group<seen in FIGS. 44 through 46(B). Spleen bioluminescence was significantly decreased in KD+HBO2T mice (p=0.0266).

TABLE 10
Treatment group cohort size and mean survival times. KD mice exhibited
a 56.7% increase in mean survival time compared to controls (p = 0.0044;
two-tailed student's t-test); KD + HBO2T mice exhibited a 77.9% increase
in mean survival time compared to controls (p = 0.0050; two-tailed
student's t-test). Results were considered significant when p < 0.05.

| | | Mean Survival Time |
|---|---|---|
| Treatment | Cohort Size (N) | (days) |
| Control (SD) | 13 | 31.2 |
| KD | 8 | 48.9 |
| SD + HBO2T | 8 | 38.8 |
| KD + HBO2T | 11 | 55.5 |

Throughout the study, health and behavior of the mice were assessed daily. Mice were humanely euthanized by CO₂ asphyxiation according to IACUC guidelines upon presentation of defined criteria (tumor-associated ascites, diminished response to stimuli, lethargy, and failure to thrive), and survival time was recorded.

KD and KD+HBO2T treated mice demonstrated a statistically different survival curve by Logrank Test with an increase in survival time compared to control animals (p=0.0194 and p=0.0035, respectively), as seen in FIG. 43. KD fed and KD+HBO2T animals also showed a significant increase in mean survival time compared to control animals by the two-tailed student's t-test (p=0.0044 and p=0.0050, respectively), seen in Table 10. Although previous studies have reported that HBO2T alone can increase survival time in animals with various cancers (Stuhr, et al. (2007) Hyperoxia retards growth and induces apoptosis, changes in vascular density and gene expression in transplanted gliomas in nude rats. Journal of neuro-oncology 85: 191-393; Stuhr et al., (2004) Hyperbaric oxygen alone or combined with 5-FU attenuates growth of DMBA-induced rat mammary tumors. Cancer letters 210: 35-75; Daruwalla & Christophi, (2006) Hyperbaric oxygen therapy for malignancy: a review. World journal of surgery 30: 2112-2143; Moen & Stuhr, (2012) Hyperbaric oxygen therapy and cancer—a review. Targeted oncology 7: 233-242), no effect was on survival in mice receiving SD+HBO2T.

Control (SD) mice had a mean survival time of 31.2 days whereas SD+HBO2T mice had a non-statistically different mean survival of 38.8 days seen in Table 10. The KD alone increased mean survival time by approximately 17 days (56.7%), and when combined with HBO2T, mice exhibited an increase in mean survival time of approximately 24 days (77.9%). This finding strongly supports the efficacy of the KD and HBO2T as therapies to inhibit tumor progression and prolong survival in animals with metastatic cancer.

Every 7 days, blood was collected from the tail using approved methods. Glucose was measured using the Nova MaxH Plus™ Glucose and b-Ketone Monitoring System (Nova Biomedical, Waltham, Mass.), and b-hydroxybutyrate was measured using the Precision Xtra™ Blood Glucose & Ketone Monitoring System (Abbott Laboratories, Abbott Park, Ill.). Mice were weighed between 1 and 3 pm twice a week for the duration of the study using the AWS-1KG Portable Digital Scale (AWS, Charleston, S.C.).

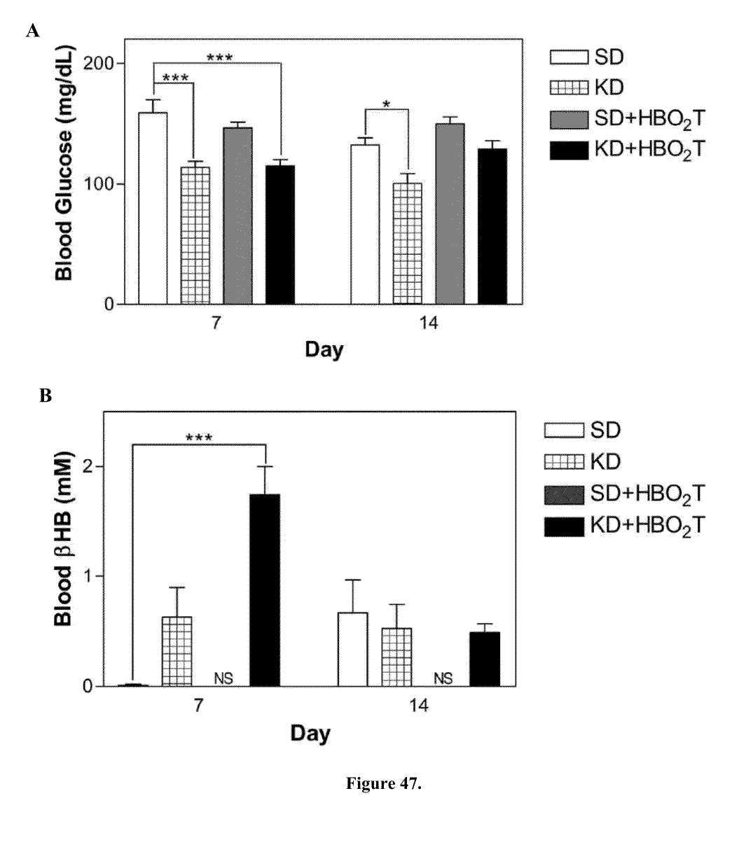

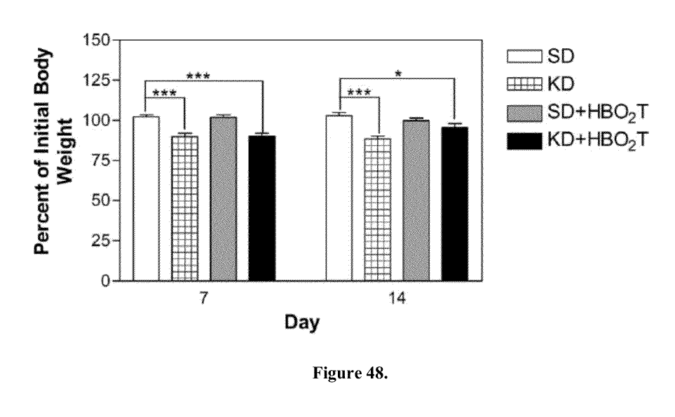

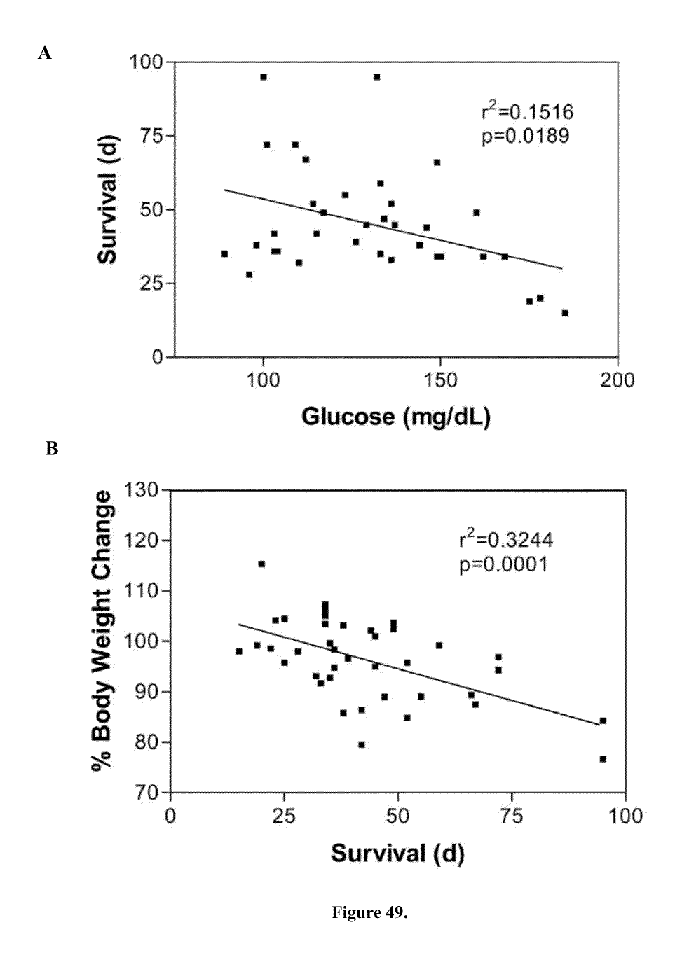

Prior to the study, initial blood glucose, ketone, and body weights were similar among the groups (data not shown). Blood glucose levels were lower in the KD-treated mice than in the SD-treated mice by day 7 (p<0.001), seen in FIGS. 47(A)-(B). While all KD-fed mice demonstrated a trend of elevated blood ketone levels throughout the duration of the study, only the KD+HBO2T animals showed significantly increased ketones compared to controls on day 7 (p<0.001). By day 7, KD-fed mice lost approximately 10% of their initial body weight and maintained that weight for the duration of the study, as seen in FIG. 48. Day 7 blood glucose and percent body weight change were significantly correlated to survival time (p=0.0189 and p=0.0001, respectively), seen in FIGS. 49(A)-(B).

Presence of intracellular ROS was measured by detection of superoxide anion (˜02) using 5 μM Dihydroethidium (DHE) following 72 hr treatment of low (5 mM) glucose, high (15 mM) glucose, or 5 mM βHB, with or without HBO2T (100% 02, 2.5 ATA, 90 min.). DHE is permeable to the plasma membrane and freely enters the cell where it reacts with .O₂⁻ to produce the oxidized ethidium. Ethidium intercalates into the DNA and fluoresces red with an excitation/emission of 485/515 nm which will be visualized using confocal fluorescent microscopy. Alternatively, ROS production can also be examined by the CellROX Deep Red Reagent (Invitrogen).

Atomic force microscopy (AFM) was utilized to analyze surface topography of VM-M3 cells in order to detect ultrastructural changes in cell morphology, such as lipid peroxidation-induced membrane blebbing (D'Agostino, et al. (2009) Acute hyperoxia increases lipid peroxidation and induces plasma membrane blebbing in human U87 glioblastoma cells. Neuroscience 159: 1011-1033; D'Agostino, et al. (2012) Development and testing of hyperbaric atomic force microscopy (AFM) and fluorescence microscopy for biological applications. Journal of microscopy 246: 129-142), following treatment with low (5 mM) glucose, high (15 mM) glucose, or 5 mM βHB. Hyperbaric atomic force microscopy (HAFM) will be similarly used to determine the effects of HBO2T (100% O₂, 2.5 ATA) on VM-M3 cell morphology.

HBO2T is known to increase ROS production in normal cells and to an even greater extent in cancer cells (Daruwalla & Christophi (2006) Hyperbaric oxygen therapy for malignancy: a review. World journal of surgery 30: 2112-2143). ROS cause oxidative stress, inducing lipid peroxidation-induced membrane blebbing which can be detected by AFM (D'Agostino, et al. (2009) Acute hyperoxia increases lipid peroxidation and induces plasma membrane blebbing in human U87 glioblastoma cells. Neuroscience 159: 1011-1033). As such VM-M3 cells should exhibit significant alterations in cell membrane morphology following HBO2T. Ketones have been shown to reduce ROS production in healthy tissues (Maalouf, et al. (2007) Ketones inhibit mitochondrial production of reactive oxygen species production following glutamate excitotoxicity by increasing NADH oxidation. Neuroscience 145: 256-264), but it is unclear if they will attenuate ROS production to the same degree in cancer cells. Mitochondrial defects of cancer should limit the ability of βHB to inhibit ROS production lipid peroxidation in the VM-M3 cells. Since glucose restriction, ketone administration, and HBO2T have been shown to inhibit cancer progression, treatments should decrease proliferation rate and reduce viability in VM-M3 cells. Since metabolic therapy and HBO2T work by overlapping mechanisms, the anti-cancer effects of low glucose and βHB treatment should be enhanced by HBO2T.

As described above, KD has a profound neuroprotective and anticonvulsant effect and is used in children to treat drug-resistant epilepsy. The literature suggests that the anticonvulsant effect of the KD depends on an elevation of a specific blood ketone (AcAc), but that βHB also provides unique neuroprotective properties. A dietary supplement of ketone esters can rapidly elevate blood ketones and significantly maintain elevated ketone levels for several hours, even higher than levels achieved with fasting, CR or KD, and without fear of metabolic acidosis associated with diabetic ketoacidosis (DKA).

The invention presented herein details the neuroprotective and anticonvulsant effect of ketone esters against CNS-OT (seizures). More specifically, it has been found that a single dose of ketone ester formulas including BD-AcAc and R BD-AcAc₂, can dramatically increase resistance to seizures (i.e. latency time to seizure) in rats exposed to hyperbaric oxygen (HBO₂; 5 ATA O₂). In addition, supplemental ketone administration prevents hyperoxia-induced oxidative stress (superoxide anion production) in cultured cortical neurons. Currently, there is no commercially-available food product or pharmaceutical that elevates ketones as significantly as ketone esters.

The inventors developed ketone esters from esters of acetoacetate (AcAc) because precursors to B-hydroxybutyrate (BHB) do not prevent CNS-OT (Chavko et al. 1999), and animal studies suggest that AcAc and acetone have the greatest anticonvulsant potential (Bough and Rho, 2007; Gasior et al., 2007; Likhodii et al., 2003; McNally and Hartman, 2011)

The inventors developed specific esters, including an enriched BD-AcAc and a purified form of BD-AcAc₂. These esters can be used alone or in mixtures. BD-AcAc is relatively water soluble, whereas BD-AcAc₂ is poorly water soluble and lipophilic.

The BD-AcAc and BD-AcAc₂ are non-ionized sodium-free precursors of the ketone body acetoacetate. When ingested these KEs are de-esterified in the blood and tissues by esterase enzymes and release acetoacetate in a rapid and sustained process. The resulting R,S-1,3 butanediol is a common food additive that breaks down to β-hydroxybutyrate. The metabolic fate of R,S-1,3 butanediol involves alcohol dehydrogenase, which catalyses the initial step in metabolism of 1,3-butanediol to β-hydroxybutyraldehyde, which is rapidly oxidized to 3-hydroxybutyrate by aldehyde dehydrogenase. Subsequent metabolic steps to acetoacetate and acetyl CoA supplies substrate for the Krebs cycle (tricarboxylic-acid cycle) to produce carbon dioxide and reducing equivalents (that are converted to ATP by the electron transport chain).

In the preceding specification, all documents, acts, or information disclosed does not constitute an admission that the document, act, or information of any combination thereof was publicly available, known to the public, part of the general knowledge in the art, or was known to be relevant to solve any problem at the time of priority.

The disclosures of all publications cited above are expressly incorporated herein by reference, each in its entirety, to the same extent as if each were incorporated by reference individually.

While there has been described and illustrated specific embodiments of ketosis and hyperbaric treatment for neurological disorders and cancers, it will be apparent to those skilled in the art that variations and modifications are possible without deviating from the broad spirit and principle of the present invention. It is intended that all matters contained in the foregoing description or shown in the accompanying drawings shall be interpreted as illustrative and not in a limiting sense. It is also to be understood that the following claims are intended to cover all of the generic and specific features of the invention herein described, and all statements of the scope of the invention which, as a matter of language, might be said to fall therebetween.

What is claimed is:

1. A method of treating metabolic dysregulation, comprising:
administering to an animal a ketogenic diet; and
subjecting the animal to a hyperbaric, oxygen-enriched environment.

2. The method of claim 1, wherein the metabolic dysregulation is Alzheimer's disease, or cancer.

3. The method of claim 1, wherein the hyperbaric, oxygen-enriched environment is 100% oxygen.

4. The method of claim 3, wherein the hyperbaric, oxygen-enriched environment is at 2.5 absolute atmosphere.

5. The method of claim 3, wherein the animal is subjected to the hyperbaric, oxygen-enriched environment for 90 minutes three times a week.

6. The method of claim 6, wherein the ketone supplementation is acetoacetate, adenosine monophosphate kinase, 1,3-butanediol, ketone ester, 1,3-butanediol acetoacetate monoester, 1,3-butanediol acetoacetate diester, MCT oil, or R,S-1,3-butanediol-diacetoacetate ester.

7. The method of claim 6, wherein the ketone supplementation is added at 10% to 20%.

8. The method of claim 6, wherein the ketone supplementation is added at 10%.

9. The method of claim 6, wherein the ketone supplementation is added at 20%.

10. The method of claim 7, wherein the ketone ester is administered about 30 minutes prior to subjecting the animal to the hyperbaric, oxygen-enriched environment.

11. The method of claim 7, wherein the ketone ester is a combination of R,S-1,3-butanediol acetoacetate monoester and R,S-1,3-butanediol acetoacetate diester.

12. The method of claim 7, wherein the ketone ester is administered at 10 g/kg.

13. A method of protecting against central nervous system oxygen toxicity, convulsions, or hyperoxia-induced oxidative stress comprising:
administering a therapeutically effective dose of a acetoacetate, adenosine monophosphate kinase, 1,3-butanediol, ketone ester, 1,3-butanediol acetoacetate monoester, 1,3-butanediol acetoacetate diester, MCT oil, or R,S-1,3-butanediol-diacetoacetate ester at a predetermined time period,
administering to an animal a ketogenic diet; and
subjecting the animal to a hyperbaric, oxygen-enriched environment.

14. The method of claim 13, wherein the acetoacetate, adenosine monophosphate kinase, 1,3-butanediol, ketone ester, 1,3-butanediol acetoacetate monoester, 1,3-butanediol acetoacetate diester, MCT oil, or R,S-1,3-butanediol-diacetoacetate ester is administered about 30 minutes prior to subjecting the animal to the hyperbaric, oxygen-enriched environment.

15. The method of claim 13, wherein the hyperbaric, oxygen-enriched environment is 100% oxygen.

16. The method of claim 15, wherein the hyperbaric, oxygen-enriched environment is at 2.5 absolute atmosphere.

17. The method of claim 15, wherein the animal is subjected to the hyperbaric, oxygen-enriched environment for 90 minutes three times a week.

18. The method of claim 13, wherein the acetoacetate, adenosine monophosphate kinase, 1,3-butanediol, ketone ester, 1,3-butanediol acetoacetate monoester, 1,3-butanediol acetoacetate diester, MCT oil, or R,S-1,3-butanediol-diacetoacetate esteris administered at 10% to 20%.

19. The method of claim 18, wherein the acetoacetate, adenosine monophosphate kinase, 1,3-butanediol, ketone ester, 1,3-butanediol acetoacetate monoester, 1,3-butanediol acetoacetate diester, MCT oil, or R,S-1,3-butanediol-diacetoacetate esteris administered at 10%.

20. The method of claim 18, wherein the acetoacetate, adenosine monophosphate kinase, 1,3-butanediol, ketone ester, 1,3-butanediol acetoacetate monoester, 1,3-butanediol acetoacetate diester, MCT oil, or R,S-1,3-butanediol-diacetoacetate esteris administered at 20%.

21. The method of claim 13, wherein the ketone ester is administered at 10 g/kg.
